(19) United States
(12) Patent Application Publication
(10) Pub. No.: US 2013/0224110 A1
(43) Pub. Date: Aug. 29, 2013

(54) USE OF ADENOSINE RECEPTOR SIGNALING TO MODULATE PERMEABILITY OF BLOOD-BRAIN BARRIER

(71) Applicant: Margaret S. Bynoe, Ithaca, NY (US)

(72) Inventor: Margaret S. Bynoe, Ithaca, NY (US)

(73) Assignee: CORNELL UNIVERSITY, Ithaca, NY (US)

(21) Appl. No.: 13/823,266

(22) PCT Filed: Sep. 16, 2011
(86) PCT No.: PCT/US11/51935

§ 371 Date: May 14, 2013

Related U.S. Application Data
(60) Provisional application No. 61/383,628, filed on Sep. 16, 2010.

(51) Int. Cl. A61K 31/7076; A61K 31/519; A61K 45/06; A61K 31/437

(52) U.S. Cl. CPC A61K 31/7076; A61K 31/437; A61K 31/519; A61K 45/06
USPC 424/1.49; 514/303; 514/45; 514/267; 424/649; 424/94.5; 424/133.1; 514/17.7; 514/8.6; 424/85.4; 424/143.1; 424/141.1; 424/147.1; 435/375

(57) ABSTRACT

The present invention relates to a method of increasing blood brain barrier (“BBB”) permeability in a subject. This method involves administering to the subject an agent or agents which activate both of the A1 and A2A adenosine receptors. Also disclosed is a method to decrease BBB permeability in a subject. This method includes administering to the subject an agent which inhibits or blocks the A2A adenosine receptor signaling. Compositions relating to the same are also disclosed.

This application claims the benefit of U.S. Provisional Patent Application Ser. No. 61/383,628, filed Sep. 16, 2010, which is hereby incorporated by reference in its entirety.

This invention was made with government support under grant numbers K22A1057854 and R01NS063011 awarded by the National Institutes of Health. The Government has certain rights in the invention.

### FIELD OF THE INVENTION

The present invention relates to modulation of blood brain barrier permeability.

### BACKGROUND OF THE INVENTION

The barriers to blood entering the central nervous system (“CNS”) are herein collectively referred to as the blood brain barrier (“BBB”). The BBB is a tremendously tight-knit layer of endothelial cells that coats 400 miles of capillaries and blood vessels in the brain (Ransohoff et al., “Three or More Routes for Leukocyte Migration Into the Central Nervous System,” Nature Rev. Immun. 3:569-581 (2003)). The blood-brain barrier (BBB) is comprised of brain endothelial cells, which form the lumen of the brain microvasculature (see Abbott et al., “Structure and Function of the Blood-Brain Barrier,” Neurobiol. Dis. 37:13-25 (2010)). The barrier function is achieved through tight junctions between endothelial cells that regulate the extravasation of molecules and cells into and out of the central nervous system (CNS) (see Abbott et al., “Structure and Function of the Blood-Brain Barrier,” Neurobiol. Dis. 37:13-25 (2010)). The nearly impermeable junctions between BBB cells are formed by the interdigitation of about 20 different types of proteins. Molecules must enter a BBB cell through membrane-embedded protein transporters or by slipping directly through its waxy outer membrane. Once inside, foreign compounds must avoid a high concentration of metabolic enzymes and a variety of promiscuous protein pumps primed to eliminate foreign substances. Having avoided these obstacles, foreign molecules must then pass through the inner membrane of a BBB cell to finally reach the brain. These elaborate defenses allow the BBB to sequester the brain from potential harm, but the BBB also obstructs delivery of neurological drugs to a site of disease in the brain. Researchers in academia and the biotech and pharmaceutical industries are learning to bypass the BBB or allow it to let potential drugs into the brain. They are designing small drugs that can passively diffuse through the BBB or travel on nutrient transporters to get inside the brain. Others are attaching potential therapeutics designed so that the brain will unwittingly engulf them.

The endothelial cells which form the brain capillaries are different from those found in other tissues in the body (Goldstein et al., “The Blood-Brain Barrier,” Scientific American 255:74-83 (1986); Pardridge, “Receptor-Mediated Peptide Transport Through the Blood-Brain Barrier,” Endocrin. Rev. 7:314-330 (1986)). Brain capillary endothelial cells are joined together by tight intercellular junctions which form a continuous wall against the passive diffusion of molecules from the blood to the brain and other parts of the CNS. These cells are also different in that they have few pinocytic vesicles which in other tissues allow somewhat unselective transport across the capillary wall. Also lacking are continuous gaps or channels running between the cells which would allow unrestricted passage.

The blood-brain barrier functions to ensure that the environment of the brain is constantly controlled. The levels of various substances in the blood, such as hormones, amino acids, and ions, undergo frequent small fluctuations which can be brought about by activities such as eating and exercise (Goldstein et al., “The Blood-Brain Barrier,” Scientific American 255:74-83 (1986); Pardridge, “Receptor-Mediated Peptide Transport Through the Blood-Brain Barrier,” Endocrin. Rev. 7:314-330 (1986)). If the brain was not protected by the blood brain barrier from these variations in serum composition, the result could be uncontrolled neural activity.

The isolation of the brain from the bloodstream is not complete. If this were the case, the brain would be unable to function properly due to a lack of nutrients and because of the need to exchange chemicals with the rest of the body. The presence of specific transport systems within the capillary endothelial cells assures that the brain receives, in a controlled manner, all of the compounds required for normal growth and function. In many instances, these transport systems consist of membrane-associated proteins, which selectively bind and transport certain molecules across the barrier membranes. These transporter proteins are known as solute carrier transporters.

Although the BBB serves to restrict the entry of potentially toxic substances into the CNS, it poses a tremendous hurdle to the delivery of therapeutic drugs into the CNS. It has been estimated that more than 98% of small-molecule drugs less than 500 Da in size do not cross the BBB (See Pardridge, “Brain Drug Targeting: the Future of Brain Drug Development,” Cambridge University Press, Cambridge, UK (2001) and Pardridge, “The Blood-Brain Barrier: Bottleneck in Brain Drug Development,” NeuroRx 2:3-14 (2005)). Current approaches aimed at altering the BBB to permit the entry of therapeutics are either too invasive, painful, can result in permanent brain damage or result in loss of drug efficacy (See Broadwell et al., “Morphologic Effect of Dimethyl Sulfoxide on the Blood-Brain Barrier,” Science 217:164-6 (1982); Hanig et al., “Ethanol Enhancement of Blood-Brain Barrier Permeability to Catecholamines in Chicksm,” Eur. J. Pharmacol. 18:79-82 (1972); Rapoport, “Advances in Osmotic Opening of the Blood-Brain Barrier to Enhance CNS Chemotherapy,” Expert Opin. Investig. Drugs 10:1809-18 (2001); Bidros et al., “Novel Drug Delivery Strategies in Neuro-Oncology,” Neurotherapeutics 6: 539-46 (2009); and Hynynen, “MRI-guided Focused Ultrasound Treatments,” Ultrasonics 50:221-9 (2010)).

Current strategies for CNS drug-delivery fall into three broad categories: chemical or physical BBB disruption and drug modification (Pardridge, “The Blood-Brain Barrier Bottleneck in Brain Drug Development,” NeuroRx 2:3-14 (2005)). Methods for chemically disrupting the BBB vary. Hypertonic mannitol osmotically shrinks brain endothelial cells, thus increasing BBB permeability and facilitating CNS delivery of chemotherapeutics (Neuwelt et al., “Osmotic Blood-brain Barrier Disruption: A New Means of Increasing Chemotherapeutic Agent Delivery,” Trans Am. Neurol. Assoc. 104:256-260 (1979)). However, it has been demonstrated that this procedure carries the risk of inducing epileptic seizures (Neuwelt et al., “Osmotic Blood-brain Barrier Modification: Clinical Documentation by Enhanced CT Scanning and/or Radionuclide Brain Scanning,” Am. J. Roentgenol. 141:829-835 (1983); Marchi et al., “Seizure-promoting Effect of Blood-brain Barrier Disruption,” Epilepsia 48:732-742 (2007)). An analogue of the vasoactive peptide bradykinin was shown to increase permeability of the blood-tumor barrier (Raymond et al., “Pharmacological Modification of Bradykinin Induced Breakdown of the Blood-brain Barrier,” Can. J. Neurol. Sci. 13:214-220 (1986)), and to some extent the BBB (Borlongan & Emerich, “Facilitation of Drug Entry into the CNS via Transient Permeation of Blood Brain Barrier: Laboratory and Preliminary Clinical Evidence from Bradykinin Receptor Agonist, Cereport,” Brain Res. Bull. 60:297-306 (2003)), and was moderately effective in increasing hydrophilic, but not lipophilic, drug delivery to certain CNS gliomas in rat models (Bartus et al., “Permeability of the Blood Brain Barrier by the Bradykinin Agonist, RMP-7: Evidence for a Sensitive, Auto-regulated, Receptor-mediated System,” Immunopharmacology 33:270-278 (1996); Elliott et al., “Intravenous RMP-7 Selectively Increases Uptake of Carboplatin into Rat Brain Tumors,” Cancer Res 56:3998-4005 (1996); Matsukado et al., “Enhanced Tumor Uptake of Carboplatin and Survival in Glioma-Bearing Rats by Intracarotid Infusion of Bradykinin Analog, RMP-7,” Neurosurgery 39:125-133, discussion 133-124 (1996); Emerich et al., “Enhanced Delivery of Carboplatin into Brain Tumours with Intravenous Cereport (RMP-7): Dramatic Differences and Insight Gained from Dosing Parameters,” Br. J. Cancer 80:964-970 (1999)). However, it failed in clinical trials, due possibly to differences between rat models and human patients (Prados et al., “A randomized, Double-blind, Placebo-controlled, Phase 2 Study of RMP-7 in Combination with Carboplatin Administered Intravenously for the Treatment of Recurrent Malignant Glioma,” Neuro. Oncol. 5:96-103 (2003)).

Physical disruption of the barrier is the oldest and most invasive method of by-passing a functional BBB. Direct injections into the brain, especially into the ventricles, have been used for years to deliver therapeutics to the CNS (Cook et al., “Intracerebroventricular Administration of Drugs,” Pharmacotherapy 29:832-845 (2009)). Recently, high-intensity focused ultrasound technologies have been developed that forcefully push therapeutic compounds past the BBB using compression waves (Bradley, “MR-guided Focused Ultrasound: A Potentially Disruptive Technology,” J. Am. Coll. Radiol. 6:510-513 (2009)). Still, physically disrupting the BBB remains invasive.

Drugs that do not cross the BBB can sometimes be modified to allow them to cross. The addition of moieties that increase a drug's lipophilicity can increase the likelihood it will cross the BBB, but these additions also render the drug more capable of entering all cell types (Witt et al., “Peptide Drug Modifications to Enhance Bioavailability and Blood-brain Barrier Permeability,” Peptides 22:2329-2343 (2001)). It is also often the case that the chemical additions themselves significantly increase the size of the drug which counteracts the higher lipophilic profile (Witt et al., “Peptide Drug Modifications to Enhance Bioavailability and Blood-brain Barrier Permeability,” Peptides 22:2329-2343 (2001)). Another approach involves so-called “vector-based” technologies in which the drug is attached to a compound known to enter the CNS through receptor-mediated endocytosis. For example, conjugation of neuronal growth factor (NGF) to a monoclonal antibody to the transferrin receptor, expressed on BECs, greatly increased NGF delivery to rat brains (Granholm et al., “NGF and Anti-transferrin Receptor Antibody Conjugate: Short and Long-term Effects on Survival of Cholinergic Neurons in Intraocular Septal Transplants,” J. Pharmacol. Exp. Ther. 268:448-459 (1994)). Vector-based delivery technologies suffer from two large drawbacks: 1) the BBB transport ability is limited to receptor expression and 2) endocytotic events are limited in BBB endothelium, a hallmark of its physiology.

There is a monumental need to modulate the BBB to facilitate the entry of therapeutic drugs into the CNS. Determining how to safely and effectively do this could affect a very broad range of neurological diseases, such as Alzheimer's disease (AD), Parkinson's disease, multiple sclerosis, neurological manifestations of acquired immune deficiency disorder (AIDS), CNS tumors, and many more. Promising therapies are available to treat some of these disorders, but their potential cannot be fully realized due to the tremendous impediment posed by the BBB. Accordingly, there is need in the art for methods to improve the delivery of compounds into the CNS.

In addition, patients suffering from edema, brain traumas, stroke and multiple sclerosis exhibit a breakdown of the BBB near the site of primary insults. The level of breakdown can have profound effects on the clinical outcome of these diseases. For instance, the degree of BBB breakdown in patients suffering from multiple sclerosis (MS) is correlated to the severity of the disease. It has been shown using Magnetic Resonance Imaging (MRI) that, when a person is undergoing an MS “attack,” the blood-brain barrier has broken down in a section of the brain or spinal cord, allowing white blood cells called T lymphocytes to cross over and destroy the myelin.

Despite the importance of this barrier, very little is known about the molecular mechanisms controlling the integrity and/or permeability of the BBB. Thus, there remains a considerable need for compositions and methods that facilitate such research and especially for diagnostic and/or therapeutic applications.

The present invention is directed to overcoming these and other deficiencies in the art.

### SUMMARY OF THE INVENTION

The present invention relates to a method for increasing blood brain barrier permeability in a subject. This method involves administering to the subject an agent which activates both of A1 and A2A adenosine receptors.

The present invention also relates to a method for increasing blood brain barrier permeability in a subject. This method involves administering to said subject an A1 adenosine receptor agonist and an A2A adenosine receptor agonist.

The present invention further relates to a composition. The composition includes an A1 adenosine receptor agonist and an A2A adenosine receptor agonist, and a pharmaceutically acceptable carrier, excipient, or vehicle.

The present invention also relates to a method for delivering a macromolecular therapeutic agent to the brain of a subject. This method includes administering to the subject an agent which activates both of A1 and A2A adenosine receptors and the macromolecular therapeutic agent.

The present invention also relates to a method for treating a CNS disease, disorder, or condition in a subject. This method involves administering to the subject at least one agent which activates both of A1 and A2A adenosine receptors and a therapeutic agent.

The present invention also relates to a method for treating a CNS disease, disorder, or condition in a subject. This method involves administering to the subject an A1 adenosine receptor agonist, an A2A receptor agonist, and a therapeutic agent.

The present invention further relates to a method of temporarily increasing the permeability of the blood brain barrier of a subject. The method comprises selecting a subject in need of a temporary increase in permeability of the blood brain barrier, providing an agent which activates either the A1 or the A2A adenosine receptor, and administering to the selected subject either the A1 or the A2A adenosine receptor agonist under conditions effective to temporarily increase the permeability of the blood brain barrier.

The present invention also relates to a method for decreasing blood brain barrier permeability in a subject. This method involves administering to said patient an agent which blocks or inhibits A2A signaling.

The present invention also relates to a method of remodeling an actin cytoskeleton of a blood brain barrier endothelial cell. This method involves contacting said endothelial cell with an agent which activates both of A1 and A2A adenosine receptors.

As shown in the examples that follow, it is demonstrated that signaling through receptors for the purine nucleoside adenosine acts as a potent endogenous modulator of blood-brain barrier permeability. These findings indicate that adenosine receptor (“AR”) signaling represents a novel endogenous mechanism for controlling BBB permeability and a potentially useful alternative to existing CNS drug-delivery technologies. Drugs like Lexiscan, the FDA-approved A2A AR agonist, which increases BBB permeability and facilitates CNS entry of macromolecules like dextrans, represent a possible pathway toward future therapeutic applications in humans. Importantly, the present findings indicate that this technique can be used for CNS delivery of macromolecular therapeutics like antibodies, which traditionally have been limited in their use in treating neurological diseases because they required invasive delivery technologies (Thakker et al., “Intracerebroventricular Amyloid-beta Antibodies Reduce Cerebral Amyloid Angiopathy and Associated Micro-hemorrhages in Aged Tg2576 Mice,” Proc. Natl. Acad. Sci. USA 106:4501-6 (2009), which is hereby incorporated by reference in its entirety). The results described here represent a novel and promising alternative to existing CNS drug-delivery paradigms.

The methods and agents of the present invention provide for an improved treatment of subjects with disorders affecting the blood brain barrier. In addition, the present invention provides improved methods of controlling the blood brain barrier to enhance therapeutic treatment of such patients.

### BRIEF DESCRIPTION OF THE DRAWINGS

FIG. 1 shows a graph demonstrating cd73^{-/-} mice are resistant to Experimental Autoimmune Encephalomyelitis (“EAE”). EAE was induced, disease activity was monitored daily, and the mean EAE score was calculated for cd73^{-/-} (open diamonds, n=11) and wild type (cd73^{+/+}) (closed squares, n=13) mice. The results shown are representative of 11 separate experiments.

FIGS. 2A-2D show cd73^{-/-} T cells produce elevated levels of IL-1β and IL-17 and mediate EAE susceptibility when transferred to cd73^{+/+}tcrα^{-/-} mice. FIG. 2A shows the CD4 and FoxP3 expression measured on splenocytes from naïve and day 13 post-EAE induced cd73^{-/-} and wild type mice. FIG. 2B shows splenocytes from naïve and day 13 post-MOG immunized wild type mice which were analyzed for CD4 and CD73 cell surface expression by flow cytometry. FIG. 2C shows sorted cells from immunized wild type or cd73^{-/-} mice which were cultured with 1×10⁴ irradiated splenocytes and 0 or 10 μM MOG peptide. Supernatants were taken at 18 hours and run on a cytokine Bio-plex assay. Results represent the fold change in cytokine levels between the 0 and 10 μM MOG peptide groups. Samples were pooled from 4 mice and are representative of one out of three similar experiments. FIG. 2D shows CD4⁺ T cells from the spleen and lymph nodes from MOG immunized cd73^{-/-} (open diamonds, n=5) or wild type (closed squares, n=5) mice which were adoptively transferred into T cell deficient cd73^{+/+}tcrα^{-/-} mice. EAE was induced and disease progression was monitored daily. Results are representative of two separate experiments.

FIG. 3A-3L show cd73^{-/-} mice which display little or no CNS lymphocyte infiltration following EAE induction; donor cd73^{-/-} T cells infiltrate the CNS of cd73^{+/+}tcrα^{-/-} recipient mice following EAE induction. Frozen tissue sections from day 13 post-EAE induction wild type (FIGS. 3A-3C) and cd73^{-/-} (FIGS. 3D-3F) mice were labeled with a CD4 antibody. FIG. 3G is a bar graph showing the mean number of CD4⁺ infiltrating lymphocytes in the brain and spinal cord quantified per field in frozen tissue sections from day 13 post-EAE induction wild type and cd73^{-/-} mice. Eight anatomically similar fields per brain and 4 fields per spinal cord per mouse were analyzed at 10× magnification (n=5 mice/group). Error bars represent the standard error of the mean. FIGS. 3H-3L show frozen tissue sections of hippocampus (FIGS. 3H, 3I, and 3K) and cerebellum (FIGS. 3J and 3L) labeled with a CD4 antibody from EAE-induced tcrα^{-/-} mice that received CD4⁺ cells from wild type (FIGS. 3H-J) or cd73^{-/-} (FIGS. 3K-3L) mice at day 12 (FIG. 3K), 18 (FIGS. 3H and 3L), or 22 (FIGS. 3I and 3J) post-EAE induction. Immunoreactivity was detected with HRP anti-rat Ig plus AEC (red) against a hemotoxylin stained nuclear background (blue). Arrows indicate sites of lymphocyte infiltration. Scale bars represent 500 μm.

FIGS. 4A-4K show cd73^{-/-} mice which display little or no CNS lymphocyte infiltration following EAE induction; cd73^{-/-} T cells infiltrate the CNS after transfer to cd73^{+/+}tcrα^{-/-} mice and EAE induction. Frozen tissue sections from day 13 post-EAE induction wild type (FIGS. 4A-4C) and cd73^{-/-} (FIGS. 4D-4F) mice were labeled with a CD45 antibody. Frozen tissue sections of hippocampus (FIGS. 4G, 4H, and 4J) and cerebellum (FIGS. 4I and 4K) labeled with a CD45 antibody from EAE-induced tcrα^{-/-} mice that received CD4⁺ cells from wild type (FIG. 4G-4I) or cd73^{-/-} (FIGS. 4J-4K) mice at day 12 (FIG. 4J), day 18 (FIGS. 4G and 4K), or day 22 (FIGS. 4H and 4I) post EAE induction. Immunoreactivity was detected with HRP anti-rat Ig plus AEC (red) against a hemotoxylin stained nuclear background (blue). Arrows indicate sites of lymphocyte infiltration. Scale bars represent 500 mm.

FIGS. 5A-5C show myelin specific T cells do not efficiently enter the brain of cd73^{-/-} mice following EAE induction. Vβ11⁺ T cells from MOG₃₅₋₅₅ immunized transgenic 2d2 mice, which express TCRs specific for MOG₃₅₋₅₅, were isolated from the spleen and lymph nodes and adoptively transferred into wild type or cd73^{-/-} mice with concomitant EAE induction. At days 1, 3, 8, and 15 post transfer and EAE induction, spleens (FIG. 5A), lymph nodes (FIG. 5B), and brains (FIG. 5C) were removed and cells were harvested. Cells were analyzed for CD45 and Vβ11 expression by flow cytometry. The data represent the relative fold change (RFC) in the percentage of Vβ11⁺ cells in the CD45⁺ population for each organ on each given day. Values were normalized to the percentage of cells found in each organ at 1 day post transfer/EAE induction, with 1.0 equaling the baseline value.

FIGS. 6A-6D show adoptively transferred CD73⁺ T cells from wild type mice can confer EAE susceptibility to cd73^{-/-} mice. FIG. 6A shows CD4⁺ T cells from the spleen and lymph nodes of MOG immunized wild type mice were enriched and adoptively transferred into wild type (closed squares, n=5) or cd73^{-/-} (open diamonds, n=5) mice followed by concomitant EAE induction. Results are shown from one of two independent experiments. FIG. 6B shows T cells from the spleen and lymph nodes of previously immunized wild type and cd73^{-/-} mice were sorted based on CD4 and CD73 expression and adoptively transferred into cd73^{-/-} mice followed by concomitant EAE induction (n=5/each group). Closed squares represent donor cells from wild type mice that express CD73; open squares represent donor cells from wild type mice that lack CD73 expression; open diamonds represent donor cells from cd73^{-/-} mice. FIG. 6C-6D show frozen tissue sections of the CNS choroid plexus from naïve wild type (FIG. 6C, left) and cd73^{-/-} (FIG. 6C, right) mice and wild type mice day 12 post-EAE induction (FIG. 6D) were stained with a CD73 (FIG. 6C) or CD45 (FIG. 6D) specific antibody. Immunoreactivity was detected with HRP anti-rat Ig plus AEC (red) against a hemotoxylin stained nuclear background (blue). Brackets indicate CD73 staining. Arrows indicate CD45 lymphocyte staining. Scale bars represent 500 μm.

FIGS. 7A-7D show adenosine receptor blockade protects mice from EAE development. FIG. 7A shows mean EAE scores where EAE was induced, disease activity was monitored daily, and the mean EAE score was calculated in wild type (squares) and cd73^{-/-} (diamonds) mice given either drinking water (closed shape) alone or drinking water supplemented with 0.6 g/ml of the broad spectrum adenosine receptor antagonist caffeine (open shape). Results are from one experiment (n=5 mice per group). FIG. 7B shows adenosine receptor mRNA expression levels relative to the GAPDH housekeeping gene in the Z310 murine choroid plexus cell line. Samples were run in triplicate; error bars represent the standard error of the mean. FIG. 7C shows results after mice were treated with the A2A adenosine receptor antagonist SCH58261 at 2 mg/kg (1 mg/kg s.c. and 1 mg/kg i.p.) in 45% DMSO (closed squares, n=4 mice/group) or 45% DMSO alone (open squares, n=5 mice/group) 1 day prior to and daily up to day 30 following EAE induction. These results are representative of two experiments. FIG. 7D shows the mean number of CD4⁺ infiltrating lymphocytes in the brain and spinal cord quantified per field in frozen tissue sections from day 15 post-EAE induction in SCH58261- and DMSO-treated mice are shown. Eight anatomically similar fields per brain and 4 fields per spinal cord per mouse were analyzed at 10× magnification (n=4 mice). Error bars represent the standard error of the mean.

FIG. 8 shows the A2A adenosine receptor antagonist SCH58261 prevents ICAM-1 upregulation on the choroid plexus following EAE induction. Mice were treated with the A2A adenosine receptor antagonist SCH58261 2 mg/kg (1 mg/kg given s.c. and 1 mg/kg given i.p.) in DMSO (n=4 mice/group) or DMSO alone (n=5 mice/group) 1 day prior to and daily up to day 30 following EAE induction. These results are from one experiment. Frozen tissue sections from day 15 post-EAE induction in SCH58261 and DMSO treated mice were examined for ICAM-1 expression at the choroid plexus. WT treated DMSO (left) or SCH58261 (right) and stained for ICAM-1 (red staining, white arrows) and DAPI (blue, nuclei) at 40× magnification. Images are from 4 separate mice.

FIGS. 9A-9B demonstrate that CD73^{-/-} mice, which lack extracellular adenosine and thus cannot adequately signal through adenosine receptors, were treated with NECA, resulting in an almost five fold increase in dye migration vs. the PBS control (FIG. 9A). WT mice treated with NECA also show an increase over control mice (FIG. 9B). Pertussis was used as a positive control, as it is known to induce blood brain barrier leakiness in the mouse EAE model.

FIG. 10 shows adenosine receptor expression on the human endothelial cell line hCMEC/D3.

FIG. 11 shows results after hCMEC/D3 cells were seeded onto transwell membranes and allowed to grow to confluencey; 2×10⁶ Jurkat cells were added to the upper chamber with or without NECA (general adenosine receptor [AR] agonist), CCPA (A1 AR agonist), CGS 21860 (A2A AR agonist), or DMSO vehicle; and migrated cells were counted after 24 hours.

FIG. 12 shows results after transwell membranes were seeded with Z310 cells and allowed to grow to confluencey; 2×10⁶ Jurkat cells were added to the upper chamber with or with out NECA (n=1, general AR agonist), CCPA (n=1, A1 AR agonist), CGS 21860 (n=1, A2A AR agonist), or DMSO vehicle(n=1); and migrated cells were counted after 24 hours.

FIG. 13 shows results after hCMEC/D3 cells were grown to confluencey on 24 well plates; cells were treated with or without various concentrations of NECA (general AR agonist), CCPA (A1 AR agonist), CGS 21860 (A2A AR agonist), DMSO vehicle, or Forksolin (induces cAMP); lysis buffer was added after 15 minutes and the cells were frozen at −80 C to stop the reaction; and cAMP levels were assayed using a cAMP Screen kit (Applied Biosystems, Foster City, Calif.).

FIG. 14 shows results of female A1 adenosine receptor knockout (A1ARKO, n=5) and wild type (WT, n=5) mice that were immunized with CFA/MOG₃₅₋₅₅+PTX on Dec. 2, 2008 and scored daily for 41 days.

FIGS. 15A-15B show brains of wild type mice fed caffeine and brains from CD73^{-/-} mice fed caffeine, as measured by FITC-Dextran extravasation through the brain endothelium.

FIG. 16 shows results in graph form of FITC-Dextran extravasation across the blood brain barrier of wild type mice treated with adenosine receptor agonist, NECA, while SCH58261, the adenosine receptor antagonist inhibit FITC-Dextran extravasation.

FIG. 17 shows results of Evans Blue dye extravasation across the blood brain barrier, as measured by a BioTex spectrophotometer at 620 nm, after mice were treated with adenosine receptor agonist NECA.

FIG. 18 shows results in graphical form that demonstrate PEGylated adenosine deaminase (“PEG-ADA”) treatment inhibits the development of EAE in wild-type mice. EAE was induced, disease activity was monitored daily, and mean EAE score was calculated in wild-type mice given either control PBS vehicle alone or 15 units/kg body weight of PEG-ADA i.p. every 4 days. Closed squares represent wild-type mice given PBS vehicle (n=3); open squares represent wild-type mice given PEG-ADA (n=3). These results are from one experiment. These results demonstrate that adenosine deaminase treatment and adenosine receptor blockade protect wild type mice against EAE induction.

FIGS. 19A-19B are bar graphs of results showing dose-dependent increases in 10,000 Da (FIG. 19A) and 70,000 Da (FIG. 19B) dextrans into WT mouse brain 3 h after i.v. administration of NECA or vehicle (DMSO/PBS) as measured by fluorimetry (10-15 animals/group). Inset in FIG. 19A is a splined scatter plot of data points. Experiments were performed at least twice. Significant differences (Student's T-test) from vehicle are indicated (*) where P≦0.05. Data are mean±s.e.m. These results demonstrate that i.v.-administered NECA increases BBB permeability to high molecular weight dextrans.

FIGS. 20A-20B show experimental results in graphical form of NECA-mediated increase in BBB permeability. FIG. 20A left panel shows extravasation of 10,000 Da FITC-dextran into WT mouse brain when co-administered with NECA or vehicle (DMSO/PBS). Gray bars=vehicle, black bars=NECA. FIG. 20A right panel is a splined scatter plot with scaled time on the x-axis, which shows an extravasation time-course of 10 kDa FITC-dextran into WT mouse brain when co-administered i.v. with NECA (0.08 mg/kg) or vehicle, as measured by fluorimetry (10-15 animals/group). FIG. 20B left panel shows the results of extravasation of 70,000 Da Texas Red-dextran into WT mouse brain tissue when injected at indicated times after NECA or vehicle administration. Gray bars=vehicle, black bars=NECA. FIG. 20B right panel is a splined scatter plot with scaled time on the x-axis, which shows extravasation time-course of 10 kDa Texas Red-dextran, administered i.v. 90 minutes prior to harvest times (as displayed), into WT mouse brain tissue after i.v. pre-treatment (time=0) with NECA (0.08 mg/kg) or vehicle, as measured by fluorimetry (3-5 animals/group). Experiments were performed at least twice. Significant differences (Student's T-test) from vehicle are indicated (*) where P≦0.05. Data are mean±s.e.m. Insets in FIGS. 20A and 20B, left panels, are splined scatter plots with scaled time on the x-axis; diamonds=vehicle, squares=NECA. These results demonstrate that NECA treatment increases BBB permeability in a temporally discrete and reversible manner.

FIGS. 21A-21J illustrate results that show that increased BBB permeability depends on selective agonism of A1 and A2A adenosine receptors. FIG. 21A is a bar graph showing relative expression of adenosine receptor subtypes on cultured mouse brain endothelial cells (“BEC”)(bEnd.3). FIG. 21B shows images of immunofluorescent staining and FIG. 21C shows images of fluorescence in situ hybridization of CD31 (endothelial cell marker; green) and A1 (left column; red) and A2A (right column; red) ARs near the cortical area of the brain in naïve mice (scale bar=20 μm). FIG. 21D shows an image of western blot analysis of A1 AR (left panel) and A2A AR (right panel) expression in isolated primary BECs from naïve mice. β-actin expression is shown as a loading control. FIGS. 21E and 21F are bar graphs showing levels of 10,000 Da FITC-dextran in WT and A1 AR (FIG. 21E) and A2A AR (FIG. 21F) knock-out mouse brain 3 h after i.v. administration of NECA or vehicle (DMSO/PBS), as measured by fluorimetry. Gray bars=vehicle, black bars=NECA. FIG. 21G is a bar graph showing decreased levels of dextran in brains of A1 and A2A AR knock-out mouse brain 3 h after i.v. administration of NECA (0.08 mg/kg) or vehicle compared with WT mice, as measured by fluorimetry. No significant increase in dextran levels were detected in brains of A1 knock-out mice that were pre-treated with the selective A2A antagonist SCH 58261 (5-8 animals/group). Also shown are data demonstrating dose-dependent entry of 10,000 Da FITC-dextran into WT brain tissue 3 h after i.v. co-administration of the specific A2A AR agonist CGS 21860 (bar graphs of FIG. 21H) or the specific A1 AR agonist CCPA (bar graphs of FIG. 21I), as measured by fluorimetry. FIG. 21J shows bar graphs illustrating levels of 10,000 Da FITC-dextran in WT mouse brain tissue 3 h after i.v. administration of vehicle, NECA, CCPA, CGS 21680 and in combination. n=3-4 mice/treatment group. Experiments were repeated at least twice. Significant differences (Student's T-test) from vehicle are indicated (*) where P≦0.05. Data are mean±s.e.m.

FIGS. 22A-22F show results in graphical form demonstrating that the A2A agonist Lexiscan increases BBB permeability to 10,000 Da dextrans. FIG. 22A shows results in graphical form that demonstrate Lexiscan administration increases BBB permeability in mice. Data bars before the axis break represent groups that received 3 Lexiscan injections. The bar after the axis break represents a group that received a single Lexiscan injection. For the groups receiving 3 injections, perfusion occurred 15 min after the initial injection. The group that received a single injection was perfused 5 min after injection (10-15 animals/group). Vehicle treated mice (V) were perfused 15 min after injection. FIG. 22B shows Lexiscan increases BBB permeability in rats. Animals received 3 injections of Lexiscan, 5 min apart, and were perfused 15 min after the initial injection (3-4 animals/group). FIG. 22C shows the results in graphical form of BBB permeability in rats to FITC-dextran administered simultaneously with 1 μg of Lexiscan at 5 minutes. As a control reference, animals received 1 injection of NECA, and were perfused 15 min after injection. Vehicle treated mice (V) were perfused 15 min after injection. Statistics indicate significant differences from vehicle (*) or from 0.01 μg Lexiscan (**), P≦0.05 by Student's T-test. Data are mean±s.e.m. FIG. 22D is a graph showing the time-course of BBB permeability after Lexiscan treatment in mice. Lexiscan (0.05 mg/kg) was administered at Time 0 (10-14 animals/group). FIG. 22E is a graph showing the time-course of BBB permeability after Lexiscan treatment in rats. Lexiscan (0.0005 mg/kg) was administered at Time 0 (3-4 animals/group). FIG. 22F shows i.p. administered SCH 58261 decreases BBB permeability to 10,000 Da FITC-dextran in mice. All experiments were repeated at least twice. Significant differences (Student's T-test) from vehicle are indicated (*) where P≦0.05. Data are mean±s.e.m.

FIGS. 23A-23H show results demonstrating that i.v.-administered antibody to β-amyloid antibody crosses BBB and labels β-amyloid plaques in transgenic mouse brains after NECA administration. FIGS. 23A-23D are immunofluorescent microscopic images near the hippocamppi of transgenic AD (APP/PSEN) mice. Mice were treated with either NECA (0.08 mg/kg) (FIGS. 23A and 23C) or vehicle (FIGS. 23B and 23D) and antibody to β-amyloid (6E10) was administered i.v. (top panels: FIGS. 23A and 23B). For mice that did not receive i.v. 6E10 antibody (lower panels: FIGS. 23C and 23D), 6E10 was used as a primary antibody to control for the presence of plaques and was applied ex vivoduring immunostaining. FIG. 23A shows the same immunofluorescent microscopic images of hippocamppi of transgenic AD (APP/PSEN) as shown in FIGS. 23A-23D, as well as those of WT mice treated with i.v.-administered antibody to β-amyloid (Covance 6E10) or not and with 0.8 μg i.v. NECA (left panels) or vehicle (right panels). In FIGS. 23A-23E, blue=DAPI and red=Cy5-antibody labeling 6E10-labeled β-amyloid plaques (scale bar=50 μm). FIGS. 23F and 23G are immunofluorescent microscopic images of the hippocampal and cortical regions from the brains of transgenic AD mice showing an overview (FIG. 23F) and close-up (FIG. 23G) of β-amyloid plaque locations relative to blood vessels (endothelial cells=CD31 stained green; β-amyloid plaques=6E10 stained red; nuclei=DAPI stained blue; scale bars=50 μm). FIG. 23H is a bar graph showing quantification of 6E10-labeled amyloid plaques per mouse brain section in transgenic AD mice treated with NECA or vehicle alone.

FIGS. 24A-24Y show results deominstrating that adenosine receptor signaling results in changes in the paracellular but not transcellular pathway on BECs. FIG. 24A is a bar graph showing relative genetic expression of adenosine receptor subtypes on cultured mouse BECs (Bend.3). FIG. 24B shows western blot analysis of A1 (left panel) and A2A (right panel) AR expression in cultured mouse BECs (Bend.3). FIG. 24C is a graph showing results that demonstrate that AR activation decreases TEER in mouse BEC monolayers. Decreased transendothelial electrical resistance was observed after addition of NECA (1 μM) or Lexiscan (1 μM) treatment. Significant differences (Student's T-test) from vehicle for Lexiscan (#) and NECA (*) are indicated where P≦0.05. Data are mean±s.e.m. FIGS. 24D-24G are images of Bend.3 cells that were incubated with fluorescently labeled albumin and either media alone (FIG. 24D), vehicle (FIG. 24E), NECA (1 μM) (FIG. 24F), or Lexiscan (1 μM) (FIG. 24G) for 30 minutes. Albumin uptake was visualized by fluorescence microscopy (albumin=red; DAPI stained nuclei=blue). Scale bar=50 μm. FIG. 24H is a bar graph showing albumin uptake results. Albumin uptake is displayed as relative values compared to the media alone control (set to 100%). Data are mean±s.e.m (n=5 fields/group). FIGS. 24I-24P are images showing results that actinomyosin stress fiber formation correlates with AR activation in cultured BECs. Phalloidin staining of Bend.3 cells is shown and reveals increased actinomyosin stress fiber formation following treatment with CCPA (1 μM) (FIGS. 24M and 24N) or Lexiscan (1 μM) (FIGS. 24O and 24P) when compared with media (FIGS. 24I and 24J) or vehicle alone (FIGS. 24K and 24L). Left panels=3 min treatment; right panels=30 min treatment. Scale bar=50 μm. FIGS. 24Q-24Y are images showing results that demonstrate that AR activation induces changes in tight junction adhesion molecules in cultured BECs. ZO-1 (FIGS. 24Q-24S), Claudin-5 (FIGS. 24T-24V), and Occludin (FIGS. 24W-24Y) staining of Bend.3 cells is shown following 1 hr treatment with DMSO (left column), NECA (1 μM, middle column), and Lexiscan (1 μM, right column). Adhesion molecules=pink/red; DAPI stained nuclei=blue. Arrow heads indicate examples of descrete changes in expression (scale bar=20 μm).

FIG. 25 is a schematic showing a model of adenosine receptor signaling and modulation of BBB permeability. (i) Basal conditions favor a tight barrier. (ii) Activation of the A1 or A2A AR results in increased BBB permeability. (iii) Activation of both A1 and A2A ARs results in even more permeability than observed after activation of either receptor alone. (iv) A2A receptor antagonism decreases BBB permeability.

### DETAILED DESCRIPTION OF THE INVENTION

Adenosine is a cellular signal of metabolic distress being produced in hypoxic, ischaemic, or inflammatory conditions. Its primary undertaking is to reduce tissue injury and promote repair by different receptor-mediated mechanisms, including the increase of oxygen supply/demand ratio, preconditioning, anti-inflammatory effects and stimulation of angiogenesis (Jacobson et al., “Adenosine Receptors as Therapeutic Targets,” Nat. Rev. Drug Discov. 5:247-264 (2006), which is hereby incorporated by reference in its entirety).

The biological effects of adenosine are ultimately dictated by the different pattern of receptor distribution and/or affinity of the four known adenosine receptor (“AR”) subtypes in specific cell types. Four AR subtypes are expressed in mammals: A1, A2A, A2B and A3 (Sebastiao et al., “Adenosine Receptors and the Central Nervous System,” Handb. Exp. Pharmacol. 471-534 (2009), which is hereby incorporated by reference in its entirety). Adenosine receptors are now known to be integral membrane proteins which bind extracellular adenosine, thereby initiating a transmembrane signal via specific guanine nucleotide binding proteins known as G-proteins to modulate a variety of second messenger systems, including adenylyl cyclase, potassium channels, calcium channels and phospholipase C. See Stiles, “Adenosine Receptors and Beyond: Molecular Mechanisms of Physiological Regulation,” Clin. Res. 38(1):10-18 (1990); Stiles, “Adenosine Receptors,” J. Biol. Chem. 267: 6451-6454 (1992), which are hereby incorporated by reference in their entirety.

The first clues to adenosine's involvement in CNS barrier permeability came from the recent findings demonstrating that extracellular adenosine, produced by the catalytic action of CD73 (a 5′-ectonucleotidase) from adenosine monophosphate (AMP), promotes leukocyte entry into the CNS in experimental autoimmune encephalomyelitis (EAE) (Mills et al., “CD73 is Required for Efficient Entry of Lymphocytes Into the Central Nervous System During Experimental Autoimmune Encephalomyelitis,” Proc. Natl. Acad. Sci. U.S.A. 105: 9325-30 (2008), which is hereby incorporated by reference in its entirety). These studies demonstrated that mice lacking CD73 (Thompson et al., “Crucial Role for Ecto-5′-nucleotidase (CD73) in Vascular Leakage During Hypoxia,” J. Exp. Med. 200:1395-405 (2004), which is hereby incorporated by reference inits entirety), which are unable to produce extracellular adenosine, are protected from EAE and that blockade of the A_{2A} adenosine receptor (AR) blocks T cell entry into the CNS (Mills et al., “CD73 is Required for Efficient Entry of Lymphocytes Into the Central Nervous System During Experimental Autoimmune Encephalomyelitis,” Proc. Natl. Acad. Sci. U.S.A. 105: 9325-30 (2008), which is hereby incorporated by reference in its entirety). Furthermore, in a pilot experiment, it was observed that after intravenous (i.v.) injection of fluorescein isothiocyanate (FITC)-labeled 10,000 Da dextran, CD73^{-/-} mice had much less FITC-dextran in their brains compared to WT mice; treatment with the broad spectrum AR agonist 5′-N-ethylcarboxamido adenosine (NECA) resulted in a dramatic increase in FITC-dextran extravasation in these mice compare to WT mice (data not shown). These observations led to the hypothesis that modulation of adenosine receptor signaling at BECs might modulate BBB permeability to facilitate the entry of molecules into the CNS. As is demonstrated in the Examples that follow, AR signaling represents a novel, endogenous modulator of BBB signaling.

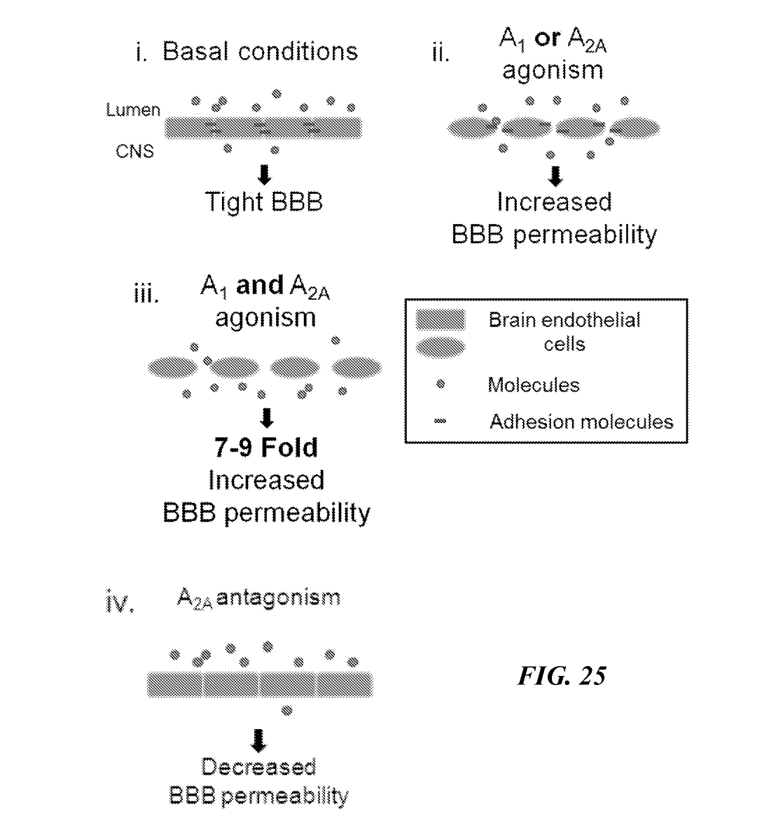

As suprisingly shown here, the activation of the A1 and the A2A adenosine receptors increases the BBB permeability of a subject. In particular, adenosine, acting through the A1 or A2A receptors, can modulate BBB permeability to either facilitate or restrict the entry of molecules into the CNS. These changes in BBB permeability are dose-dependent and temporally discrete. Given that adenosine has a relatively short half-life, ˜<10 seconds (Klabunde, “Dipyridamole Inhibition of Adenosine Metabolism in Human Blood,” Eur. J. Pharmacol. 93:21-6 (1983), which is hereby incorporated by reference in its entirety), its role as a physiologic modulator is probably limited to the local environment in which it is produced. Indeed, the expression of CD39 and CD73 with adenosine receptors on brain endothelial cells indicates these cells have the ability to respond to extracellular ATP, a well-established damage signal (Davalos et al., “ATP Mediates Rapid Microglial Response to Local Brain Injury in vivo,” Nat. Neurosci. 8:752-8 (2005) and Haynes et al., “The P2Y12 Receptor Regulates Microglial Activation by Extracellular Nucleotides,” Nat. Neurosci. 9:1512-9 (2006), which are hereby incorporated by reference in their entirety). Adenosine receptor signaling at BBB endothelial cells is a key event in the “sensing” of damage that would necessitate changes in barrier permeability, and BBB permeability (mediated through A1 and A2A ARs) operates as a door where activation opens the door, antagonism closes the door and local adenosine concentration is the key. The absence of elevated levels of extracellular adenosine favors a tight and restrictive barrier. As shown schematically in FIG. 25, activation of either the A1 or A2A AR temporarily increases BBB permeability, while activation of both receptors results in an additive effect of increased BBB permeability. It is shown here that BBB permeability mediated through A1 and A2A ARs operates as a door where activation opens the door and local adenosine concentration is the key.

One aspect of the present invention is directed to a method for increasing blood brain barrier permeability in a subject. This method involves administering to the subject an agent which activates both of A1 and A2A adenosine receptors.

It will be understood by those of skill in the art that the barrier between the blood and central nervous system is made up of the endothelial cells of the blood capillaries (blood-brain barrier (“BBB”)) and by the epithelial cells of the choroid plexus (“CP”) that separate the blood from the cerebrospinal fluid (“CSF”) of the central nervous system (“CNS”). Together these structures function as the CNS barrier.

In one embodiment, the methods of the present invention for increasing BBB permeability, increase the permeability of the CP. In another embodiment, the methods of the present invention for increasing the permeability of the BBB, increase the permeability of the CNS barrier.

In one embodiment, the method further involves selecting a subject in need of increased BBB permeability, providing a therapeutic, and administering to the selected subject the therapeutic and an agent which activates both of A1 and A2A adenosine receptors under conditions effective for the therapeutic to cross the blood brain barrier.

A suitable subject in need of increased permeability of the BBB according to the present invention includes any subject that is in need of a therapeutic to cross the BBB to treat or prevent a disease, disorder, or condition of the CNS or that which manifests within the CNS (e.g., HIV-associated neurological disorders).

It will be understood that a therapeutically effective amount of the agents according to the present invention is administered. The terms “effective amount” and “therapeutically effective amount,” as used herein, refer to the amount of a compound or combination that, when administered to an individual, is effective to treat, prevent, delay, or reduce the severity of a condition from which the patient is suffering. In particular, a therapeutically effective amount in accordance with the present invention is an amount sufficient to treat, prevent, delay onset of, or otherwise ameliorate at least one side-effect associated with the treatment of a disease and/or disorder.

Suitable A1 and/or A2A adenosine receptor activators according to the present invention include agonists that are selective for the A1 adenosine receptor, agonists that are selective for the A2A adenosine receptor, agonists that activate both the A1 and the A2A adenosine receptors, broad spectrum adenosine activators or agonists, and combinations thereof. According to certain embodiments of the present invention a combination of the A1-selective agonist, A2A-selective agonist, an agonist that activates both the A1 and the A2A adenosine receptors, and/or broad spectrum adenosine activators or agonists are administered. These agents may be administered simultaneously, in the same or different pharmaceutical formulation, or sequentially. The timing of the sequential administration can be determined by a skilled practitioner. In certain embodiments, the agonists are combined in a single unit dosage form.

Suitable A2A adenosine receptor activators are A2A agonists, which are well known in the art (Press et al., “Therapeutic Potential of Adenosine Receptor Antagonists and Agonists,” Expert Opin. Ther. Patents 17(8): 979-991 (2007), which is hereby incorporated by reference in its entirety). Examples of A2A adenosine receptor agonists include those described in U.S. Pat. No. 6,232,297 and in U.S. Published Patent Application No. 2003/0186926 A1 to Lindin et al., 2005/0054605 A1 to Zablocki et al., and U.S. Published Patent Application Nos. 2006/0040888 A1, 2006/0040889 A1, 2006/0100169 A1, and 2008/0064653 A1 to Li et al., which are hereby incorporated by reference in their entirety. Such compounds may be synthesized as described in: U.S. Pat. Nos. 5,140,015 and 5,278,150 to Olsson et al.; U.S. Pat. No. 5,593,975 to Cristalli; U.S. Pat. No. 4,956,345 Miyasaka et al.; Hutchinson et al., “CGS 21680C, an A2 Selective Adenosine Receptor Agonist with Preferential Hypotensive Activity,” J. Pharmacol. Exp. Ther., 251: 47-55 (1989); Olsson et al, “N6-Substituted N-alkyladenosine-5′-uronamides: Bifunctional Ligands Having Recognition Groups for A1 and A2 Adenosine Receptors,” J. Med. Chem., 29: 1683-1689 (1986); Bridges et al., “N6-[2-(3,5-dimethoxyphenyl)-2-(2-methylphenyl)ethyl]adenosine and its Uronamide Derivatives: Novel Adenosine Agonists With Both High Affinity and High Selectivity for the Adenosine A2 Receptor,” J. Med. Chem. 31: 1282 (1988); Hutchinson et al., J. Med. Chem., 33:1919 (1990); Ukeeda et al., “2-Alkoxyadenosines: Potent and Selective Agonists at the Coronary Artery A2 Adenosine Receptor,” J. Med. Chem. 34: 1334 (1991); Francis et al., “Highly Selective Adenosine A2 Receptor Agonists in a Series of N-alkylated 2-aminoadenosines,” J. Med. Chem. 34: 2570-2579 (1991); Yoneyama et al, “Vasodepressor Mechanisms of 2-(1-octynyl)-adenosine (YT-146), a Selective Adenosine A2 Receptor Agonist, Involve the Opening of Glibenclamide-sensitive K+ Channels,” Eur. J. Pharmacol. 213(2):199-204 (1992); Peet et al., “Conformationally Restrained, Chiral (phenylisopropyl)amino-substituted pyrazolo[3,4-d]pyrimidines and Purines with Selectivity for Adenosine A1 and A2 Receptors,” J. Med. Chem., 35: 3263-3269 (1992); and Cristalli et al., “2-Alkynyl Derivatives of Adenosine and Adenosine-5′-N-ethyluronamide as Selective Agonists at A2 Adenosine Receptors,” J. Med. Chem. 35(13): 2363-2368 (1992), which are hereby incorporated by reference in their entirety. Additional examples of adenosine A2A receptor agonists are disclosed in U.S. Patent Application Publication 2004/0809916, which is hereby incorporated by reference in its entirety. Particularly suitable A2A adenosine receptor agonists include 4-[2-[[6-Amino-9-(N-ethyl-b-D-ribofuranuronamidosyl)-9H-purin-2-yl]amino]ethyl]benzenepropanoic acid (“CGS 21680”), and Lexiscan, or combinations thereof. These adenosine A2A receptor agonists are intended to be illustrative and not limiting.

Suitable A1 adenosine receptor activators are A1 adenosine receptor agonists. A1 adenosine receptor agonists are known to those of skill in the art and include, for example, those described in U.S. Patent Application Publication No. 2005/0054605 A1 to Zablocki et al. and Press et al., “Therapeutic Potential of Adenosine Receptor Antagonists and Agonists,” Expert Opin. Ther. Patents 17(8): 979-991 (2007), which are hereby incorporated by reference in their entireties. Suitable A1 adenosine receptor agonists also include, for example, 2-chloro-N⁶-cyclopentyladenosine (“CCPA”), 8-cyclopentyl-1,3-dipropylxanthine (“DPCPX”), R-phenylisopropyl-adenosine, N6-Cyclopentyladenosine, and N(6)-cyclohexyladenosine, or combinations thereof.

In one embodiment, the agent which activates both the A1 and the A2A adenosine receptors is an agonist of both the A1 and the A2A adenosine receptors. Suitable agonists that activate both the A1 and the A2A adenosine receptors are known to those of skill in the art, and include, for example, AMP 579. In still further embodiments, the agonist of both the A1 and the A2A adenosine receptors may be a broad spectrum adenosine receptor agonist. Suitable broad spectrum adenosine receptor agonists will be known to those of skill in the art and include, for example, NECA, adenosine, adenosine derivatives, or combinations thereof.

According to one embodiment of the present invention, activating both the A1 and A2A adenosine receptors is synergistic as compared to the level of BBB permeability when activating either the A1 adenosine receptor or A2A adenosine receptor alone. In this context, if the effect of activating the two receptors together (at a given concentration) is greater than the sum of the effects when each receptor is activated individually (at the same concentration), then the activation of both the A1 and the A2A receptors is considered to be synergistic.

In a further embodiment, activation of both the A1 and the A2A adenosine receptors increases BBB permeability by 2, 3, 4, 5, 6, 7, 8, 9, or 10 fold, or any range encompassed therein. In one embodiment, activating both the A1 adenosine receptor and the A2A adenosine receptor increases the BBB permeability 7-9 fold.

According to certain embodiments of the present invention, the activation of both the A1 and the A2A receptors is additive. In this context, if the effect of activating the two receptors together (at a given concentration) is equivalent to the sum of the effects when each receptor is activated individually (at the same concentration), then the activation of both the A1 and the A2A receptors together is considered to be additive.

In one embodiment according to the present invention, the increase in BBB permeability lasts up to 18 hours. In further embodiments, the increase in BBB permeability lasts up to about 17 hours, 16 hours, 15 hours, 14 hours, 13 hours, 12 hours, 11 hours, 10 hours, 9 hours, 8 hours, 6 hours, 4 hours, 3 hours, 2 hours, 1 hour, 30 minutes, 15 minutes, 10 minutes, or 5 minutes.

Another aspect of the present invention relates to increasing blood brain barrier permeability in a subject. This method includes administering to the subject an A1 adenosine receptor agonist and an A2A adenosine receptor agonist.

In one embodiment, the A1 adenosine receptor agonist and/or the A2A adenosine receptor agonist are selective agonists. As used herein, “selective” means having an activation preference for a specific receptor over other receptors which can be quantified based upon whole cell, tissue, or organism assays which demonstrate receptor activity.

Suitable A1-selective receptor agonists according to the present invention include 2-chloro-N⁶-cyclopentyladenosine (“CCPA”), N6-Cyclopentyladenosine, N(6)-cyclohexyladenosine, 8-cyclopentyl-1,3-dipropylxanthine (“DPCPX”), R-phenylisopropyl-adenosine, or combinations thereof.

Suitable A2A-selective receptor agonists according to the present invention include Lexiscan (also known as Regadenoson), CGS 21680, ATL-146e, YT-146 (2-(1-octynyl)adenosine), DPMA (N6-(2-(3,5-dimethoxyphenyl)-2-(2-methylphenyl)ethyl)adenosine), or combinations thereof.

In one embodiment, the A1 adenosine receptor agonist and the A2A adenosine receptor agonist may be administered simultaneously. In another embodiment according to the present invention, the A1 adenosine receptor agonist and the A2A adenosine receptor agonist may be administered sequentially.

In certain embodiments, the A1 adenosine receptor agonist and the A2A adenosine receptor agonist are formulated in a single unit dosage form. Dosage and formulations according to the present invention are described in further detail below.

In one embodiment, this method further includes the administration of a therapeutic agent. The therapeutic agent may be administered together with one or both of the A1 adenosine receptor agonist and the A2A adenosine receptor agonist, or may be administered following administration of the A1 adenosine receptor agonist and/or the A2A adenosine receptor agonist. Suitable therapeutic agents are described in further detail below. In certain embodiments, the agonists may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent.

Another aspect of the present invention relates to a composition. The composition includes an A1 adenosine receptor agonist, an A2A adenosine receptor agonist, and a pharmaceutically acceptable carrier, excipient, or vehicle.

In one embodiment according to this aspect of the present invention, the A1 adenosine receptor agonist and/or the A2A adenosine receptor agonist are selective agonists.

The compounds, compositions, or agents of the present invention can be administered locally or systemically. In particular the compounds, compositions, or agents of the present invention can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active compounds or agents of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained. A convenient unitary dosage formulation contains the active ingredients in amounts from 0.1 mg to 1 g each, for example 5 mg to 500 mg. Typical unit doses may, for example, contain about 0.5 to about 500 mg, or about 1 mg to about 500 mg of an agent according to the present invention.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds or agents may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or agents of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

In one embodiment, the composition according to the present invention includes a therapeutic agent. In a further embodiment, the therapeutic is suitable for treating a central nervous system (“CNS”) disease, disorder, or condition. Such therapeutic agents are well known in the art and many are common and typically prescribed agents for a relevant disorder. Dosage ranges for such agents are known to one of ordinary skill in the art and are often found in the accompanying prescription information pamphlet (often referred to as the “label”).

Disorders of the CNS (which encompass psychiatric/behavioral diseases or disorders) may include, but are not limited to, acquired epileptiform aphasia, acute disseminated encephalomyelitis, adrenoleukodystrophy, agenesis of the corpus callosum, agnosia, aicardi syndrome, Alexander disease, Alpers' disease, alternating hemiplegia, Alzheimer's disease, amyotrophic lateral sclerosis, anencephaly, Angelman syndrome, angiomatosis, anoxia, aphasia, apraxia, arachnoid cysts, arachnoiditis, Arnold-chiari malformation, arteriovenous malformation, Asperger's syndrome, ataxia telangiectasia, attention deficit hyperactivity disorder, autism, auditory processing disorder, autonomic dysfunction, back pain, Batten disease, Behcet's disease, Bell's palsy, benign essential blepharospasm, benign focal amyotrophy, benign intracranial hypertension, bilateral frontoparietal polymicrogyria, binswanger's disease, blepharospasm, Bloch-sulzberger syndrome, brachial plexus injury, brain abscess, brain damage, brain injury, brain tumor, spinal tumor, Brown-séquard syndrome, canavan disease, carpal tunnel syndrome (cts), causalgia, central pain syndrome, central pontine myelinolysis, centronuclear myopathy, cephalic disorder, cerebral aneurysm, cerebral arteriosclerosis, cerebral atrophy, cerebral gigantism, cerebral palsy, charcot-marie-tooth disease, chiari malformation, chorea, chronic inflammatory demyelinating polyneuropathy (“CIDP”), chronic pain, chronic regional pain syndrome, Coffin lowry syndrome, coma (including persistent vegetative state), congenital facial diplegia, corticobasal degeneration, cranial arteritis, craniosynostosis, Creutzfeldt-jakob disease, cumulative trauma disorders, Cushing's syndrome, cytomegalic inclusion body disease (“CIBD”), cytomegalovirus infection, dandy-walker syndrome, Dawson disease, de morsier's syndrome, Dejerine-klumpke palsy, Dejerine-sottas disease, delayed sleep phase syndrome, dementia, dermatomyositis, developmental dyspraxia, diabetic neuropathy, diffuse sclerosis, dysautonomia, dyscalculia, dysgraphia, dyslexia, dystonia, early infantile epileptic encephalopathy, empty sella syndrome, encephalitis, encephalocele, encephalotrigeminal angiomatosis, encopresis, epilepsy, Erb's palsy, erythromelalgia, essential tremor, Fabry's disease, Fahr's syndrome, fainting, familial spastic paralysis, febrile seizures, fisher syndrome, Friedreich's ataxia, Gaucher's disease, Gerstmann's syndrome, giant cell arteritis, giant cell inclusion disease, globoid cell leukodystrophy, gray matter heterotopia, Guillain-barré syndrome, htlv-1 associated myelopathy, Hallervorden-spatz disease, head injury, headache, hemifacial spasm, hereditary spastic paraplegia, heredopathia atactica polyneuritiformis, herpes zoster oticus, herpes zoster, hirayama syndrome, holoprosencephaly, Huntington's disease, hydranencephaly, hydrocephalus, hypercortisolism, hypoxia, immune-mediated encephalomyelitis, inclusion body myositis, incontinentia pigmenti, infantile phytanic acid storage disease, infantile refsum disease, infantile spasms, inflammatory myopathy, intracranial cyst, intracranial hypertension, Joubert syndrome, Kearns-sayre syndrome, Kennedy disease, kinsbourne syndrome, Klippel feil syndrome, Krabbe disease, Kugelberg-welander disease, kuru, lafora disease, Lambert-eaton myasthenic syndrome, Landau-kleffner syndrome, lateral medullary (Wallenberg) syndrome, learning disabilities, leigh's disease, Lennox-gastaut syndrome, Lesch-nyhan syndrome, leukodystrophy, lewy body dementia, lissencephaly, locked-in syndrome, Lou Gehrig's disease, lumbar disc disease, lyme disease—neurological sequelae, machado-joseph disease (spinocerebellar ataxia type 3), macrencephaly, megalencephaly, Melkersson-rosenthal syndrome, Ménière's disease, meningitis, Menkes disease, metachromatic leukodystrophy, microcephaly, migraine, Miller Fisher syndrome, mini-strokes, mitochondrial myopathies, mobius syndrome, monomelic amyotrophy, motor neurone disease, motor skills disorder, moyamoya disease, mucopolysaccharidoses, multi-infarct dementia, multifocal motor neuropathy, multiple sclerosis, multiple system atrophy with postural hypotension, muscular dystrophy, myalgic encephalomyelitis, myasthenia gravis, myelinoclastic diffuse sclerosis, myoclonic encephalopathy of infants, myoclonus, myopathy, myotubular myopathy, myotonia congenita, narcolepsy, neurofibromatosis, neuroleptic malignant syndrome, neurological manifestations of aids, neurological sequelae of lupus, neuromyotonia, neuronal ceroid lipofuscinosis, neuronal migration disorders, niemann-pick disease, non 24-hour sleep-wake syndrome, nonverbal learning disorder, O'sullivan-mcleod syndrome, occipital neuralgia, occult spinal dysraphism sequence, ohtahara syndrome, olivopontocerebellar atrophy, opsoclonus myoclonus syndrome, optic neuritis, orthostatic hypotension, overuse syndrome, palinopsia, paresthesia, Parkinson's disease, paramyotonia congenita, paraneoplastic diseases, paroxysmal attacks, parry-romberg syndrome (also known as rombergs syndrome), pelizaeus-merzbacher disease, periodic paralyses, peripheral neuropathy, persistent vegetative state, pervasive developmental disorders, photic sneeze reflex, phytanic acid storage disease, pick's disease, pinched nerve, pituitary tumors, pmg, polio, polymicrogyria, polymyositis, porencephaly, post-polio syndrome, postherpetic neuralgia (“PHN”), postinfectious encephalomyelitis, postural hypotension, Prader-willi syndrome, primary lateral sclerosis, prion diseases, progressive hemifacial atrophy (also known as Romberg's syndrome), progressive multifocal leukoencephalopathy, progressive sclerosing poliodystrophy, progressive supranuclear palsy, pseudotumor cerebri, ramsay-hunt syndrome (type I and type II), Rasmussen's encephalitis, reflex sympathetic dystrophy syndrome, refsum disease, repetitive motion disorders, repetitive stress injury, restless legs syndrome, retrovirus-associated myelopathy, rett syndrome, Reye's syndrome, Romberg's syndrome, rabies, Saint Vitus' dance, Sandhoff disease, schizophrenia, Schilder's disease, schizencephaly, sensory integration dysfunction, septo-optic dysplasia, shaken baby syndrome, shingles, Shy-drager syndrome, Sjögren's syndrome, sleep apnea, sleeping sickness, snatiation, Sotos syndrome, spasticity, spina bifida, spinal cord injury, spinal cord tumors, spinal muscular atrophy, spinal stenosis, Steele-richardson-olszewski syndrome, see progressive supranuclear palsy, spinocerebellar ataxia, stiff-person syndrome, stroke, Sturge-weber syndrome, subacute sclerosing panencephalitis, subcortical arteriosclerotic encephalopathy, superficial siderosis, sydenham's chorea, syncope, synesthesia, syringomyelia, tardive dyskinesia, Tay-sachs disease, temporal arteritis, tetanus, tethered spinal cord syndrome, Thomsen disease, thoracic outlet syndrome, tic douloureux, Todd's paralysis, Tourette syndrome, transient ischemic attack, transmissible spongiform encephalopathies, transverse myelitis, traumatic brain injury, tremor, trigeminal neuralgia, tropical spastic paraparesis, trypanosomiasis, tuberous sclerosis, vasculitis including temporal arteritis, Von Hippel-lindau disease (“VHL”), Viliuisk encephalomyelitis (“VE”), Wallenberg's syndrome, Werdnig-hoffman disease, west syndrome, whiplash, Williams syndrome, Wilson's disease, and Zellweger syndrome. It is thus appreciated that all CNS-related states and disorders could be treated through the BBB route of drug delivery.

A CNS disease, disorder, or condition according to embodiments of the present invention may be selected from a metabolic disease, a behavioral disorder, a personality disorder, dementia, a cancer, a neurodegenerative disorder, pain, a viral infection, a sleep disorder, a seizure disorder, acid lipase disease, Fabry disease, Wernicke-Korsakoff syndrome, ADHD, anxiety disorder, borderline personality disorder, bipolar disorder, depression, eating disorder, obsessive-compulsive disorder, schizophrenia, Alzheimer's disease, Barth syndrome and Tourette's syndrome, Canavan disease, Hallervorden-Spatz disease, Huntington's disease, Lewy Body disease, Lou Gehrig's disease, Machado-Joseph disease, Parkinson's disease, or Restless Leg syndrome.

In one embodiment, the CNS disease, disorder, or condition is pain and is selected from neuropathic pain, central pain syndrome, somatic pain, visceral pain, and/or headache.

Suitable CNS therapeutics according to the present invention include small molecule therapeutic agents. Suitable small molecule therapeutics for treating a disease, disorder, or condition of the CNS include acetaminophen, acetylsalicylic acid, acyltransferase, alprazolam, amantadine, amisulpride, amitriptyline, amphetamine-dextroamphetamine, amsacrine, antipsychotics, antivirals, apomorphine, arimoclomol, aripiprazole, asenapine, aspartoacyclase enzyme, atomoxetine, atypical antipsychotics, azathioprine, baclofen, beclamide, benserazide, benserazide-levodopa, benzodiazepines, benztropine, bevacizumab, bleomycin, brivaracetam, bromocriptine, buprenorphine, bupropion, cabergoline, carbamazepine, carbatrol, carbidopa, carbidopa-levodopa, carboplatin, chlorambucil, chlorpromazine, chlorprothixene, cisplatin, citalopram, clobazam, clomipramine, clonazepam, clozapine, codeine, COX-2 inhibitors, cyclophosphamide, dactinomycin, dexmethylphenidate, dextroamphetaine, diamorphine, diastat, diazepam, diclofenac, donepezil, doxorubicin, droperidol, entacapone, epirubicin, escitalopram, ethosuximide, etoposide, felbamate, fluoxetine, flupenthixol, fluphenazine, fosphenytoin, gabapentin, galantamine, gamma hydroxybutyrate, gefitinib, haloperidol, hydantoins, hydrocordone, hydroxyzine, ibuprofen, ifosfamide, IGF-1, iloperidone, imatinib, imipramine, interferons, irinotecan, KNS-760704, lacosamide, lamotrigine, levetiracetam, levodopa, levomepromazine, lisdexamfetamine, lisuride, lithium carbonate, lypolytic enzyme, mechlorethamine, mGluR2 agonists, memantine, meperidine, mercaptopurine, mesoridazine, mesuximide, methamphetamine, methylphenidate, minocycline, modafinil, morphine, N-acetylcysteine, naproxen, nelfinavir, neurotrin, nitrazepam, NSAIDs, olanzapine, opiates, oseltamivir, oxaplatin, paliperidone, pantothenate kinase 2, Parkin, paroxetine, pergolide, periciazine, perphenazine, phenacemide, phenelzine, phenobarbitol, phenturide, phenyloin, pimozide, Pink1, piribedil, podophyllotoxin, pramipexole, pregabalin, primidone, prochlorperazine, promazine, promethazine, protriptyline, pyrimidinediones, quetiapine, rasagiline, remacemide, riluzole, risperidone, ritonavir, rituximab, rivastigmine, ropinirole, rotigotine, rufinamide, selective serotonin reuptake inhibitors (SSRIs), selegine, selegiline, sertindole, sertraline, sodium valproate, stiripentol, taxanes, temazepam, temozolomide, tenofovir, tetrabenazine, thiamine, thioridazine, thiothixene, tiagabine, tolcapone, topiramate, topotecan, tramadol, tranylcypromine, trastuzumab, tricyclic antidepressants, trifluoperazine, triflupromazine, trihexyphenidyl, trileptal, valaciclovir, valnoctamide, valproamide, valproic acid, venlafaxine, vesicular stomatitis virus, vigabatrin, vinca alkaloids, zanamivir, ziprasidone, zonisamide, zotepine, zuclopenthixol, or combinations thereof.

In another embodiment, the composition according to the present invention may include a therapeutic agent suitable for treatment of human immunodeficiency virus (“HIV”). The agent chosen from nucleoside HIV reverse transcriptase inhibitors, non-nucleoside HIV reverse transcriptase inhibitors, HIV protease inhibitors, HIV integrase inhibitors, HIV fusion inhibitors, immune modulators, CCR5 antagonists, and antiinfectives.

Pathogens such as HIV seek refuge in the CNS where they can remain for the life of the host. More than 30 million people world-wide are currently infected with HIV and these numbers are likely to increase (See United Nations: Report on The Global AIDS Epidemic (2008), which is hereby incorporated by reference in its entirety). Without an effective method of getting anti-HIV drugs into the CNS to target the virus, it seems unlikely that HIV will ever be eradicated.

Other therapeutic agents or compounds that may be administered according to the present invention may be of any class of drug or pharmaceutical agent which is desirable to cross the BBB. Such therapeutics include, but not limited to, antibiotics, anti-parasitic agents, antifungal agents, anti-viral agents and anti-tumor agents. When administered with anti-parasitic, anti-bacterial, anti-fungal, anti-tumor, anti-viral agents, and the like, the compounds according to the present invention may be administered by any method and route of administration suitable to the treatment of the disease, typically as pharmaceutical compositions.

Therapeutic agents can be delivered as a therapeutic or as a prophylactic (e.g., inhibiting or preventing onset of neurodegenerative diseases). A therapeutic causes eradication or amelioration of the underlying disorder being treated. A prophylactic is administered to a patient at risk of developing a disease or to a patient reporting one or more of the physiological symptoms of such a disease, even though a diagnosis may not have yet been made. Alternatively, prophylactic administration may be applied to avoid the onset of the physiological symptoms of the underlying disorder, particularly if the symptom manifests cyclically. In this latter embodiment, the therapy is prophylactic with respect to the associated physiological symptoms instead of the underlying indication. The actual amount effective for a particular application will depend, inter alia, on the condition being treated and the route of administration.

The therapeutic may be selected from the group consisting of immunosuppressants, anti-inflammatories, anti-proliferatives, anti-migratory agents, anti-fibrotic agents, proapoptotics, calcium channel blockers, anti-neoplasties, antibodies, anti-thrombotic agents, anti-platelet agents, Ilblllla agents, antiviral agents, anti-cancer agents, chemotherapeutic agents, thrombolytics, vasodilators, antimicrobials or antibiotics, antimitotics, growth factor antagonists, free radical scavengers, biologic agents, radio therapeutic agents, radio-opaque agents, radiolabelled agents, anti-coagulants (e.g., heparin and its derivatives), anti-angiogenesis drugs (e.g., Thalidomide), angiogenesis drugs, PDGF-B and/or EGF inhibitors, anti-inflammatories (e.g., psoriasis drugs), riboflavin, tiazofurin, zafurin, anti-platelet agents (e.g., cyclooxygenase inhibitors (e.g., acetylsalicylic acid)), ADP inhibitors (such as clopidogrel and ticlopdipine), hosphodiesterase III inhibitors (such as cilostazol), lycoprotein II/IIIIa agents (such as abcix-imab), eptifibatide, and adenosine reuptake inhibitors (such as dipyridmoles, healing and/or promoting agents (e.g., anti-oxidants and nitrogen oxide donors)), antiemetics, antinauseants, tripdiolide, diterpenes, triterpenes, diterpene epoxides, diterpenoid epoxide, triepoxides, or tripterygium wifordii hook F(TWHF), SDZ-RAD, RAD, RAD666, or 40-0-(2-hydroxy)ethyl-rapamycin, derivatives, pharmaceutical salts and combinations thereof.

In certain embodiments, the therapeutic and the adenosine receptor activator agent(s) (or adenosine receptor blockers or inhibitor, as described in further detail below) and/or therapeutics are formulated as a single “compound” formulation. This can be accomplished by any of a number of known methods. For example, the therapeutic agent and the activator agent can be combined in a single pharmaceutically acceptable excipient. In another approach the therapeutic and the adenosine receptor activator (or adenosine receptor blocker or inhibitor) agent can be formulated in separate excipients that are microencapsulated and then combined, or that form separate laminae in a single pill, and so forth.

In one embodiment, the therapeutic and adenosine receptor activator agent are linked together. In certain embodiments, the therapeutic and the adenosine receptor activator (or adenosine receptor blocker or inhibitor) agent are joined directly together or are joined together by a “tether” or “linker” to form a single compound. Without being bound to a particular theory, it is believed that such joined compounds provide improved specificity/selectivity.

A number of chemistries for linking molecules directly or through a linker/tether are well known to those of skill in the art. The specific chemistry employed for attaching the therapeutic(s) and the adenosine receptor activator (or adenosine receptor blocker or inhibitor) agent to form a bifunctional compound depends on the chemical nature of the therapeutic(s) and the “interligand” spacing desired. Various therapeutics and adenosine receptor activator agents typically contain a variety of functional groups (e.g., carboxylic acid (COOH), free amine (—NEE), and the like), that are available for reaction with a suitable functional group on a linker or on the opposing component (i.e., either the therapeutic or adenosine receptor activator) to bind the components together.

Alternatively, the components can be derivatized to expose or attach additional reactive functional groups. The derivatization may involve attachment of any of a number of linker molecules such as those available from Pierce Chemical Company, Rockford Ill.

A “linker” or “tether”, as used herein, is a molecule that is used to join two or more ligands (e.g., therapeutic(s) or adenosine receptor activator) to form a bi-functional or poly-functional compound. The linker is typically chosen to be capable of forming covalent bonds to all of the components comprising the bi-functional or polyfunctional moiety. Suitable linkers are well known to those of skill in the art and include, but are not limited to, straight or branched-chain carbon linkers, heterocyclic carbon linkers, amino acids, nucleic acids, dendrimers, synthetic polymers, peptide linkers, peptide and nucleic acid analogs, carbohydrates, polyethylene glycol and the like. Where one or more of the components are polypeptides, the linker can be joined to the constituent amino acids through their side groups (e.g., through a disulfide linkage to cysteine) or through the alpha carbon amino or carboxyl groups of the terminal amino acids.

In certain embodiments, a bifunctional linker having one functional group reactive with a group on the first therapeutic and another group reactive with a functional group on the adenosine receptor activator agent can be used to form a bifunctional compound. Alternatively, derivatization may involve chemical treatment of the component(s) (e.g., glycol cleavage of the sugar moiety of a glycoprotein, a carbohydrate, or a nucleic acid, etc.) with periodate to generate free aldehyde groups. The free aldehyde groups can be reacted with free amine or hydrazine groups on a linker to bind the linker to the compound (See, e.g., U.S. Pat. No. 4,671,958 to Rodwell et al., which is hereby incorporated by reference in its entirety). Procedures for generation of free sulfhydryl groups on polypeptide, such as antibodies or antibody fragments, are also known (See U.S. Pat. No. 4,659,839 to Nicolotti et al., which is hereby incorporated by reference in its entirety).

Where the therapeutic and the adenosine receptor activator agent are both peptides, a bifunctional compound can be chemically synthesized or recombinantly expressed as a fusion protein comprising both components attached directly to each other or attached through a peptide linker.

In certain embodiments, lysine, glutamic acid, and polyethylene glycol (PEG) based linkers of different length are used to couple the components. The chemistry for the conjugation of molecules to PEG is well known to those of skill in the art (see, e.g., Veronese, “Peptide and Protein PEGylation: a Review of Problems and Solutions,” Biomaterials 22: 405-417 (2001); Zalipsky et al., “Attachment of Drugs to Polyethylene Glycols,” Eur. Plym. J. 19(12):1177-1183 (1983); Olson et al., “Preparation and Characterization of Poly(ethylene glycol)ylated Human Growth Hormone Antagonist,” Poly(ethylene glycol) Chemistry and Biological Applications 170-181, Harris & Zalipsky Eds., ACS, Washington, D.C. (1997); Delgado et al., “The Uses and Properties of PEG-Linked Proteins,” Crit. Rev. Therap. Drug Carrier Sys. 9: 249-304 (1992); Pedley et al., “The Potential for Enhanced Tumour Localisation by Poly(ethylene glycol) Modification of anti-CEA Antibody,” Brit. J. Cancer 70:1126-1130 (1994); Eyre & Farver, Textbook of Clinical Oncology 377-390 (Holleb et al. eds. 1991); Lee et al., “Prolonged Circulating Lives of Single-chain of Fv Proteins Conjugated with Polyethylene Glycol: a Comparison of Conjugation Chemistries and Compounds,” Bioconjug. Chem. 10: 973-981 (1999); Nucci et al., “The Therapeutic Value of Poly(Ethylene Glycol)-Modified Proteins,” Adv. Drug Deliv. Rev. 6: 133-151 (1991); Francis et al., “Polyethylene Glycol Modification: Relevance of Improved Methodology to Tumour Targeting,” J. Drug Targeting 3: 321-340 (1996), which are hereby incorporated by reference in their entirety).

In certain embodiments, conjugation of the therapeutic and the adenosine receptor activator (or adenosine receptor blocker or inhibitor) agent can be achieved by the use of such linking reagents such as glutaraldehyde, EDCI, terephthaloyl chloride, cyanogen bromide, and the like, or by reductive amination. In certain embodiments, components can be linked via a hydroxy acid linker of the kind disclosed in WO-A-9317713. PEG linkers can also be utilized for the preparation of various PEG tethered drugs (See, e.g., Lee et al., “Reduction of Azides to Primary Amines in Substrates Bearing Labile Ester Functionality: Synthesis of a PEG-Solubilized, “Y”-Shaped Iminodiacetic Acid Reagent for Preparation of Folate-Tethered Drugs,” Organic Lett., 1: 179-181 (1999), which is hereby incorporated by reference in its entirety). In other embodiments, the adenosine receptor activator (or adenosine receptor blocker or inhibitor) agent) may be PEGylated (e.g., PEGylated adenosine deaminase).

Another aspect of the present invention relates to a method of delivering a macromolecule therapeutic agent to the brain of a subject. This method involves administering to the subject (a) an agent which activates both of A1 and A2A adenosine receptors and (b) the macromolecular therapeutic.

In certain embodiments, the macromolecular therapeutic agent may be a bioactive protein or peptide agent. Examples of such bioactive protein or peptides include a cell modulating peptide, a chemotactic peptide, an anticoagulant peptide, an antithrombotic peptide, an anti-tumor peptide, an anti-infectious peptide, a growth potentiating peptide, and an anti-inflammatory peptide. Examples of proteins include antibodies, enzymes, steroids, growth hormone and growth hormone-releasing hormone, gonadotropin-releasing hormone and its agonist and antagonist analogues, somatostatin and its analogues, gonadotropins, peptide T, thyrocalcitonin, parathyroid hormone, glucagon, vasopressin, oxytocin, angiotensin I and II, bradykinin, kallidin, adrenocorticotropic hormone, thyroid stimulating hormone, insulin, glucagon and the numerous analogues and congeners of the foregoing molecules. In some aspects of the invention, the BBB permeability is modulated by one or more methods herein above to deliver an antibiotic, or an anti-infectious therapeutic capable agent. Such anti-infectious agents reduce the activity of or kills a microorganism.

The nature of the peptide agent is not limited, other than comprising amino acid residues. The peptide agent can be a synthetic or a naturally occurring peptide, including a variant or derivative of a naturally occurring peptide. The peptide can be a linear peptide, cyclic peptide, constrained peptide, or a peptidomimetic. Methods for making cyclic peptides are well known in the art. For example, cyclization can be achieved in a head-to-tail manner, side chain to the N- or C-terminus residues, as well as cyclizations using linkers. The selectivity and activity of the cyclic peptide depends on the overall ring size of the cyclic peptide which controls its three dimensional structure. Cyclization thus provides a powerful tool for probing progression of disease states, as well as targeting specific self-aggregation states of diseased proteins.

In some embodiments, the peptide agent specifically binds to a target protein or structure associated with a neurological condition. In accordance with these embodiments, the invention provides agents useful for the selective targeting of a target protein or structure associated with a neurological condition, for diagnosis or therapy. Peptide agents useful in accordance with the present invention are described in, for example, U.S. Patent Application Publication 2009/0238754 to Wegrzyn et al., which is hereby incorporated by reference in its entirety.

In other embodiments, the peptide agent specifically binds to a target protein or structure associated with other neurological conditions, such as stroke, cerebrovascular disease, epilepsy, transmissible spongiform encephalopathy (TSE); Aβ-peptide in amyloid plaques of Alzheimer's disease (AD), cerebral amyloid angiopathy (CAA), and cerebral vascular disease (CVD); α-synuclein deposits in Lewy bodies of Parkinson's disease, tau in neurofibrillary tangles in frontal temporal dementia and Pick's disease; superoxide dismutase in amylotrophic lateral sclerosis; and Huntingtin in Huntington's disease and benign and cancerous brain tumors such as glioblastoma's, pituitary tumors, or meningiomas.

In some embodiments, the peptide agent undergoes a conformational shift other than the alpha-helical to beta-sheet shift discussed above, such as a beta-sheet to alpha-helical shift, an unstructured to beta-sheet shift, etc. Such peptide agents may undergo such conformational shifts upon interaction with target peptides or structures associated with a neurological condition.

In other embodiments, the peptide agent is an antibody that specifically binds to a target protein or structure associated with a neurological condition, such as a target protein or structure (such as a specific conformation or state of self-aggregation) associated with an amyloidogenic disease, such as the anti-amyloid antibody 6E10, and NG8. Other anti-amyloid antibodies are known in the art, as are antibodies that specifically bind to proteins or structures associated with other neurological conditions.

In certain embodiments, the macromolecular therapeutic agent is a monoclonal antibody. Suitable monoclonal antibodies include 6E10, PF-04360365, 131I-chTNT-1/B MAb, 131I-L19SIP, 177Lu-J591, ABT-874, AIN457, alemtuzumab, anti-PDGFR alpha monoclonal antibody IMC-3G3, astatine At 211 monoclonal antibody 81C6, Bapineuzumab, Bevacizumab, cetuximab, cixutumumab, Daclizumab, Hu MiK-beta-1, HuMax-EGFr, iodine I 131 monoclonal antibody 3F8, iodine I 131 monoclonal antibody 81C6, iodine I 131 monoclonal antibody 8H9, iodine I 131 monoclonal antibody TNT-1/B, LMB-7 immunotoxin, MAb-425, MGAWN1, Me1-14 F(ab′)2, M-T412, Natalizumab, Neuradiab, Nimotuzumab, Ofatumumab, Panitumumab, Ramucirumab, ranibizumab, SDZ MSL-109, Solanezumab, Trastuzumab, Ustekinumab, Zalutumumab, Tanezumab, Aflibercept, MEDI-578, REGN475, Muromonab-CD3, Abiximab, Rituximab, Basiliximab, Palivizumab, Infliximab, Gemtuzumab ozogamicin, Ibritumomab tiuxetan, Adalimumab, Omalizumab, Tositumomab, Tositumomab-I131, Efalizumab, Abciximab, Certolizumab pegol, Eculizumab, AMG-162, Zanolimumab, MDX-010, Anti0MRSA mAb, Pexelizumab, Mepolizumab, Epratuzumab, Anti-RSV mAb, Afelimomab, Catumaxomab, WX-G250, or combinations thereof.

In certain embodiments, the macromolecular therapeutic agent is a peptide detection agent. For example, peptide detection agents include fluorescent proteins, such as Green Flourescent Protein (GFP), streptavidin, enzymes, enzyme substrates, and other peptide detection agents known in the art.

In other embodiments, the macromolecular therapeutic agent includes peptide macromolecules and small peptides. For example, neurotrophic proteins are useful as peptide agents in the context of the methods described herein. Neurotrophic proteins include nerve growth factor (NGF), brain-derived neurotrophic factor (BDNF), neurotrophin-3 (NT-3), neurotrophin-4 (NT-4), neurotrophin-5 (NT-5), insulin-like growth factors (IGF-I and IGF-II), glial cell line derived neurotrophic factor (GDNF), fibroblast growth factor (FGF), ciliary neurotrophic factor (CNTF), epidermal growth factor (EGF), glia-derived nexin (GDN), transforming growth factor (TGF-α and TGF-β), interleukin, platelet-derived growth factor (PDGF) and S100β protein, as well as bioactive derivatives and analogues thereof.

Neuroactive peptides also include the subclasses of hypothalamic-releasing hormones, neurohypophyseal hormones, pituitary peptides, invertebrate peptides, gastrointestinal peptides, those peptides found in the heart, such as atrial naturetic peptide, and other neuroactive peptides. Hypothalamic releasing hormones include, for example, thyrotropin-releasing hormones, gonadotropin-releasing hormone, somatostatins, corticotropin-releasing hormone and growth hormone-releasing hormone. Neurohypophyseal hormones include, for example, compounds such as vasopressin, oxytocin, and neurophysins. Pituitary peptides include, for example, adrenocorticotropic hormone, β-endorphin, α-melanocyte-stimulating hormone, prolactin, luteinizing hormone, growth hormone, and thyrotropin. Suitable invertebrate peptides include, for example, FMRF amide, hydra head activator, proctolin, small cardiac peptides, myomodulins, buccolins, egg-laying hormone and bag cell peptides. Gastrointestinal peptides include, for example, vasoactive intestinal peptide, cholecystokinin, gastrin, neurotensin, methionineenkephalin, leucine-enkephalin, insulin and insulin-like growth factors I and II, glucagon, peptide histidine isoleucineamide, bombesin, motilin and secretins. Examples of other neuroactive peptides include angiotensin II, bradykinin, dynorphin, opiocortins, sleep peptide(s), calcitonin, CGRP (calcitonin gene-related peptide), neuropeptide Y, neuropeptide Yy, galanin, substance K (neurokinin), physalaemin, Kassinin, uperolein, eledoisin and atrial naturetic peptide.

In yet further embodiments, the macromolecular therapeutic agent is a protein associated with membranes of synaptic vesicles, such as calcium-binding proteins and other synaptic vesicle proteins. The subclass of calcium-binding proteins includes the cytoskeleton-associated proteins, such as caldesmon, annexins, calelectrin (mammalian), calelectrin (torpedo), calpactin I, calpactin complex, calpactin II, endonexin I, endonexin II, protein II, synexin I; and enzyme modulators, such as p65. Other synaptic vesicle proteins include inhibitors of mobilization (such as synapsin Ia,b and synapsin IIa,b), possible fusion proteins such as synaptophysin, and proteins of unknown function such as p29, VAMP-1,2 (synaptobrevin), VAT1, rab 3A, and rab 3B.

Macromolecular therapeutic agents also include α-, β- and γ-interferon, epoetin, Fligrastim, Sargramostin, CSF-GM, human-IL, TNF and other biotechnology drugs.

Macromolecular therapeutic agents also include peptides, proteins and antibodies obtained using recombinant biotechnology methods.

Macromolecular therapeutic agents also include “anti-amyloid agents” or “anti-amyloidogenic agents,” which directly or indirectly inhibit proteins from aggregating and/or forming amyloid plaques or deposits and/or promotes disaggregation or reduction of amyloid plaques or deposits. Anti-amyloid agents also include agents generally referred to in the art as “amyloid busters” or “plaque busters.” These include drugs which are peptidomimetic and interact with amyloid fibrils to slowly dissolve them. “Peptidomimetic” means that a biomolecule mimics the activity of another biologically active peptide molecule. “Amyloid busters” or “plaque busters” also include agents which absorb co-factors necessary for the amyloid fibrils to remain stable.

Anti-amyloid agents include antibodies and peptide probes, as described in PCT application PCT/US2007/016738 (WO 2008/013859) and U.S. patent application Ser. No. 11/828,953, the entire contents of which are incorporated herein by reference in their entirety. As described therein, a peptide probe for a given target protein specifically binds to that protein, and may preferentially bind to a specific structural form of the target protein. While not wanting to be bound by any theory, it is believed that binding of target protein by a peptide probe will prevent the formation of higher order assemblies of the target protein, thereby preventing or treating the disease associated with the target protein, and/or preventing further progression of the disease. For example, binding of a peptide probe to a monomer of the target protein will prevent self-aggregation of the target protein. Similarly, binding of a peptide probe to a soluble oligomer or an insoluble aggregate will prevent further aggregation and protofibril and fibril formation, while binding of a peptide probe to a protofibril or fibril will prevent further extension of that structure. In addition to blocking further aggregation, this binding also may shift the equilibrium back to a state more favorable to soluble monomers, further halting the progression of the disease and alleviating disease symptoms.

In one embodiment, the macromolecular therapeutic agent is a variant of a peptide agent described above, with one or more amino acid substitutions, additions, or deletions, such as one or more conservative amino acid substitutions, additions, or deletions, and/or one or more amino acid substitutions, additions, or deletions that further enhances the permeability of the conjugate across the BBB. For example, amino acid substitutions, additions, or deletions that result in a more hydrophobic amino acid sequence may further enhance the permeability of the conjugate across the BBB.

In another embodiment, the macromolecular therapeutic agent is about 150 kDa in size. In yet another embodiment, the therapeutic is up to about 10,000 Da in size, up to about 70,000 Da in size, or up to about 150 kDa in size. In still further embodiments the therapeutic is between about 10,000 and about 70,000 Da, between about 70,000 Da and 150 kDa, or between about 10,000 Da and about 150 kDa in size.

In one embodiment, the agent that activates both of the A1 and A2A adenosine receptors is administered before the therapeutic macromolecule. In further embodiments, the agent that activates both of the A1 and A2A adenosine receptors may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic macromolecule agent.

In another embodiment, the agent or agents that activate both of the A1 and A2A adenosine receptors is administered simultaneously with the therapeutic macromolecule.

Another aspect of the present invention relates to a method for treating a CNS disease, disorder, or condition in a subject. This method involves administering to the subject at least one agent which activates both of the A1 and the A2A adenosine receptors and a therapeutic agent.

Suitable therapeutic agents are described above and may include small molecule therapeutic agents, macromolecular therapeutic agents, or combinations thereof.

In one embodiment, the agent which activates both of the A1 and the A2A adenosine receptors is an agonist of both the A1 and the A2A adenosine receptors. In further embodiments, the agonist of both the A1 and the A2A adenosine receptors is a broad spectrum adenosine receptor agonist, such as NECA, adenosine, adenosine derivatives, or combinations thereof.

Another aspect of the present invention relates to a method of treating a CNS disease, disorder or condition in a subject. This method includes administering to the subject (a) an adenosine receptor agonist; (b) an A2A receptor agonist; and (c) a therapeutic agent.

In one embodiment according to this aspect of the present invention, the A1 adenosine receptor agonist and/or the A2A adenosine receptor agonist are selective agonists.

Suitable A1-selective adenosine receptor agonist, A2A-selective adenosine receptor agonists, and therapeutic agents (along with their preparation and administration) are noted above.

In a further embodiment, this method further involves selecting a subject in need of treatment or prevention of a CNS disease, disorder, or condition; providing a therapeutic agent; and administering to the selected subject the therapeutic, an A1 adenosine receptor agonist, and an A2A receptor agonist under conditions effective for the therapeutic to cross the blood brain barrier and treat or prevent the CNS disease, disorder or condition.

In one embodiment the A1 adenosine receptor agonist and A2A adenosine receptor agonist are formulated in a single unit dosage form.

In another embodiment the A1 adenosine receptor agonist and A2A adenosine receptor agonist are administered simultaneously.

In yet a further embodiment the A1 adenosine receptor agonist and A2A adenosine receptor agonist are administered sequentially.

In still a further embodiment, the method further includes administering a composition that includes an A1 adenosine receptor agonist and A2A adenosine receptor agonist, and a pharmaceutically acceptable carrier, excipient, or vehicle.

Another aspect of the present invention relates to a method of temporarily increasing the permeability of the blood brain barrier of a subject. This method includes selecting a subject in need of a temporary increase in permeability of the blood brain barrier, providing an agent which activates either the A1 or the A2A adenosine receptor, and administering to the selected subject either the A1 or the A2A adenosine receptor activating agent under conditions effective to temporarily increase the permeability of the blood brain barrier.

In one embodiment, the A1 or the A2A activating agent is an A1 or A2A agonist. In a further embodiment, the A1 or the A2A adenosine receptor activating agent is an A1-selective or an A2-selective adenosine receptor agonist. Suitable A1 and A2A adenosine receptor agonists are known to those of skill in the art and are described in detail above.

In a further embodiment of this aspect of the present invention, the method further includes administering a therapeutic agent to the subject. Suitable therapeutic agents are described in detail above.

In one embodiment, the agent that activates the A1 or the A2A adenosine receptor is administered before the therapeutic agent. In further embodiments, the agent that activates the A1 or the A2A adenosine receptor may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent.

In another embodiment the agent that activates the A1 or the A2A adenosine receptor and the therapeutic agent are administered simultaneously.

Another aspect of the present invention is directed to a method of decreasing BBB permeability in a subject. This method involves administering to the subject or patient an agent which blocks or inhibits A2A adenosine receptor signaling.

In decreasing BBB permeability, the selected subject can have an inflammatory disease. Such inflammatory diseases include those in which mediators of inflammation pass the blood brain barrier. Such inflammatory diseases include, but are not limited to, inflammation caused by bacterial infection, viral infection, or autoimmune disease. More specifically, such diseases include, but are not limited to, meningitis, multiple sclerosis, neuromyelitis optica, human immunodeficiency virus (“HIV”)-1 encephalitis, herpes simplex virus (“HSV”) encephalitis, Toxoplams gondii encephalitis, and progressive multifocal leukoencephalopathy.

Where BBB permeability is decreased, the selected subject may also have a condition mediated by entry of lymphocytes into the brain. Other conditions treatable in this fashion include encephalitis of the brain, Parkinson's disease, epilepsy, neurological manifestations of HIV-AIDS, neurological sequela of lupus, and Huntington's disease, meningitis, multiple sclerosis, neuromyelitis optica, HSV encephalitis, and progressive multifocal leukoencephalopathy.

This aspect of the present invention can be carried out using the pharmaceutical formulation methods and methods of administration described above.

Altering adenosine receptor activity in a subject to decrease blood barrier permeability can be accomplished by, but not limited to, deactivating or blocking the A2A adenosine receptor.

A number of adenosine A2A receptor antagonists are known to those of skill in the art and can be used individually or in conjunction in the methods described herein. Such antagonists include, but are not limited to (−)-R,S)-mefloquine (the active enantiomer of the racemic mixture marketed as Mefloquine™), 3,7-Dimethyl-1-propargylxanthine (DMPX), 3-(3-hydroxypropyl)-7-methyl-8-(m-methoxystyryl)-1-propargylxanthine (MX2), 3-(3-hy-droxypropyl)-8-(3-methoxystyryl)-7-methyl-1-propargylxanthin phosphate disodium salt (MSX-3, a phosphate prodrug of MSX-2), 7-methyl-8-styrylxanthine derivatives, SCH 58261, KW-6002, aminofuryltriazolo-tri-azinylaminoethylphenol (ZM 241385), and 8-chlorostyryl-caffeine, KF17837, VR2006, istradefylline, the VERNALIS drugs such as VER 6489, VER 6623, VER 6947, VER 7130, VER 7146, VER 7448, VER 7835, VER 8177VER-11135, VER-6409, VER 6440, VER 6489, VER 6623, VER 6947, VER 7130, VER 7146, VER 7448, VER 7835, VER 8177, pyrazolo[4,3-e]1,2,4-triazolo[1,5-c]pyrimidines, and 5-amino-imidazolo-[4,3-e]-1,2,4-triazolo[1,5-c]pyrimidines, and the like (U.S. Patent Application Publication No. 2006/0128708 to Li et al., which is hereby incorporated by reference in its entirety), pyrazolo[4,3-e]-[1,2,4]-triazolo[1,5-c]pyrimidines (See e.g., WO 01/92264 to Kase et al., which is hereby incorporated by reference in its entirety), 2,7-disubstituted-5-amino-[1,2,4]triazolo[1,5-c]pyrimidines (See e.g., WO 03/048163 to Kase et al., which is hereby incorporated by reference in its entirety), 2,5-disubstituted-7-amino-[1,2,4]triazolo[1,5-a][1,3,5]triazines (See e.g., Vu et al., “piperazine Derivatives of [1,2,4]Triazolo[1,5-a][1,3,5]triazine as Potent and Selective Adenosine A2A Receptor Antagonists,” J. Med. Chem. 47(17):4291-4299 (2004), which is hereby incorporated by reference in its entirety), 9-substituted-2-(substituted-ethyn-1-yl)-adenines (See e.g., U.S. Pat. No. 7,217,702 to Beauglehole et al., which is hereby incorporated by reference in its entirety), 7-methyl-8-styrylxanthine derivatives, pyrazolo[4,3-e)1,2,4-triazolo[1,5-c]pyrimidines, and 5-amino-imidazolo-[4,3-e]-1,2,4-triazolo[1,5-c]pyrimidines (See e.g., U.S. Patent Application Publication No. 2006/0128708 to Li et al., which is hereby incorporated by reference in its entirety). These adenosine A2A receptor antagonists are intended to be illustrative and not limiting.

Yet a further aspect of the present invention relates to a method for increasing BBB permeability followed by deacreasing BBB permeability. The method involves administration of one or more agents that activate the A1 and A2A adenosine receptors followed by administration of an agent that blocks or inhibits A2A adenosine receptor signaling.

In one embodiment, the one or more agents that activate the A1 and A2A adenosine receptors is administered simultaneously with a therapeutic agent. In another embodiment, the one or more agents that activate both the A1 and A2A adenosine receptors is administered before a therapeutic agent. In this embodiment, the agent that blocks or inhibits A2A adenosine receptor signaling is administered following administration of the therapeutic agent.

Yet another aspect of the present invention relates to a method of remodeling an actin cytoskeleton of a BBB endothelial cell. This method involves contacting an endothelial cell with one or more agents that activates both of the A1 and the A2A adenosine receptors.

The actin cytoskeleton is vital for the maintenance of cell shape. Endothelial barrier permeability can be affected by reorganization of the actin cytoskeleton. The actin cytoskeleton is organized into three distinct structures: the cortical actin rim, actomyosin stress fibers, and actin cross-linking of the membrane skeleton (Prasain et al., “The Actin Cytoskeleton in Endothelial Cell Phenotypes,” Microvasc. Res. 77:53-63 (2009), which is hereby incorporated by reference in its entirety). These structures have unique roles in controlling endothelial cell shape.

According to one embodiment, the actin cytoskeleton remodeling increases space between endothelial cells and increases BBB permeability.

Suitable A1 and A2A adenosine receptor activators are disclosed above.

In one embodiment according to this aspect of the present invention, the activation of both of the A1 and A2A adenosine receptors is synergistic with respect to BBB permeability. In yet another embodiment, the activation of both of the A1 and A2A adenosine receptors is additive with respect to BBB permeability.

While the identification of the A1 and A2A ARs as critical mediators of BBB permeability represents the first step towards a molecular mechanism, much work remains to elucidate the specific downstream players that facilitate cellular changes in the endothelial cells. Adenosine receptors are G-protein coupled receptors, associated with heterotrimeric G-proteins. Several G_{α} subunits have been localized to tight junctions (Denker et al., “Involvement of a Heterotrimeric G Protein Alpha Subunit in Tight Junction Biogenesis,” J. Biol. Chem. 271:25750-3 (1996), which is hereby incorporated by reference in its entirety). These G_{α} subunits are known to influence the activity of downstream enzymes like RhoA and Rac1 that have been implicated in cytoskeletal remodeling. Indeed, work by other groups suggests that the RhoA and Rac1 small GTPases are responsive to extracellular signaling and mediate changes in the actin cytoskeleton (Schreibelt et al., “Reactive Oxygen Species Alter Brain Endothelial Tight Junction Dynamics Via RhoA, PI3 kinase, and PKB Signaling,” Faseb J. 21:3666-76 (2007); Jou et al., “Structural and Functional Regulation of Tight Junctions by RhoA and Rac1 Small GTPases,” J Cell Biol 142, 101-15 (1998); and Wojciak-Stothard et al., “Regulation of TNF-alpha-induced Reorganization of the Actin Cytoskeleton and Cell-cell Junctions by Rho, Rac, and Cdc42 in Human Endothelial Cells,” J. Cell. Physiol. 176:150-165 (1998), which are hereby incorporated by reference in their entireties). Additionally, there is evidence that adenosine affects actin through the Rho GTPase (Sohail et al., “Adenosine Induces Loss of Actin Stress Fibers and Inhibits Contraction in Hepatic Stellate Cells via Rho Inhibition,” Hepatology 49:185-94 (2009), which is hereby incorporated by reference in its entirety). Importantly, inflammation caused by canonical damage signals like TNF-α and thrombin increases BBB permeability by altering tight junctions through cytoskeletal reorganization (Wojciak-Stothard et al., “Regulation of TNF-alpha-Induced Reorganization of the Actin Cytoskeleton and Cell-Cell Junctions by Rho, Rac, and Cdc42 in Human Endothelial Cells,” J. Cell. Physiol. 176:150-65 (1998) and Lum et al., “Mechanisms of Increased Endothelial Permeability,” Can. J. Physiol. Pharmacol. 74:787-800 (1996), which are hereby incorporated by reference in their entireties). Signaling events initiated by activation of A1 and A2A ARs on brain endothelial cells result in actin cytoskeletal remodeling which, by changing cell shape, increases the space between the endothelial cells and allows increased molecular diffusion. Adenosine has been shown to affect other endothelial cell barrier properties in a similar manner (Lu et al., “Adenosine Protected Against Pulmonary Edema Through Transporter- and Receptor A2-mediated Endothelial Barrier Enhancement,” Am. J. Physiol. Lung. Cell. Mol. Physiol. 298: L755-67 (2010), which is hereby incorporated by reference in its entirety). However, here actomyosin stress fiber formation in brain endothelial cell monolayers was observed upon A1 or A2A AR activation with specific agonists. Conversely, blockade of these receptors with AR antagonists could act in the opposite fashion and result in increased tightness between the cells. In the absence of active signaling from ARs, this model favors a tight barrier (FIG. 25). This strongly correlates AR activation with stress fiber formation. Taken together with the present observations that AR agonists also decrease TEER in BEC monolayers, it indicates that AR modulation, acting through cytoskeletal elements, causes changes in endothelial cell shape that increase barrier permeability.

### EXAMPLES

The following examples are provided to illustrate embodiments of the present invention but are by no means intended to limit its scope.

### Example 1

### Mice

Cd73^{-/-} mice have been previously described (Thompson et al., “Crucial Role for Ecto-5′-Nucleotidase (CD73) in Vascular Leakage During Hypoxia,” J. Exp. Med. 200:1395-1405 (2004), which is hereby incorporated by reference in its entirety) and have been backcrossed to C57BL/6 for 14 generations. Cd73^{-/-} mice have no overt susceptibility to infection and appear normal based on the size and cellular composition of their lymphoid organs and their T and B cell responses in in vivo and in vitro assays (Thompson et al., “Crucial Role for Ecto-5′-Nucleotidase (CD73) in Vascular Leakage During Hypoxia,” J. Exp. Med. 200:1395-1405 (2004), which is hereby incorporated by reference in its entirety). C57BL/6 and tcrα^{-/-} mice on the C57BL/6 background were purchased from The Jackson Laboratories. Mice were bred and housed under specific pathogen-free conditions at Cornell University or the University of Turku. For adenosine receptor blockade experiments, mice were given drinking water supplemented with 0.6 g/L of caffeine (Sigma) or 2 mg/kg SCH58261 (1 mg/kg s.c. and 1 mg/kg i.p.) in DMSO (45% vol. in PBS) or 45% DMSO alone starting 1 day before EAE induction and continuing throughout the experiment. All procedures performed on mice were approved by the relevant animal review committee.

### Example 2

### EAE Induction and Scoring

EAE was induced by subjecting mice to the myelin oligodendrocyte glycoprotein (“MOG”) EAE-inducing regimen as described in Swanborg, “Experimental Autoimmune Encephalomyelitis in Rodents as a Model for Human Demyelinating Disease,” Clin. Immunol. Immunopathol. 77:4-13 (1995) and Bynoe et al., “Epicutaneous Immunization with Autoantigenic Peptides Induces T Suppressor Cells that Prevent Experimental Allergic Encephalomyelitis,” Immunity 19:317-328 (2003), which are hereby incorporated by reference in their entirety. Briefly, a 1:1 emulsion of MOG₃₅₋₅₅ peptide (3 mg/ml in PBS) (Invitrogen) and complete Freund's adjuvant (CFA, Sigma) was injected subcutaneously (50 μl) into each flank. Pertussis toxin (PTX, 20 ng) (Biological Laboratories Inc.) was given intravenously (200 μl in PBS) at the time of immunization and again 2 days later. Mice were scored daily for EAE based on disease symptom severity; 0=no disease, 0.5-1=weak/limp tail, 2=limp tail and partial hind limb paralysis, 3=total hind limb paralysis, 4=both hind limb and fore limb paralysis, 5=death. Mice with a score of 4 were euthanized.

### Example 3

### T Cell Preparations and Adoptive Transfer

Mice were primed with MOG₃₅₋₅₅ peptide in CFA without PTX. After one week, lymphocytes were harvested from spleen and lymph nodes and incubated with ACK buffer (0.15M NH₄Cl, 1 mM KHCO₃, 0.1 mM EDTA, pH 7.3) to lyse red blood cells. Cells were incubated with antibodies to CD8 (TIB-105), IA^{b,d,v,p,q,r} (212.A1), FcR (2.4-G2), B220 (TIB-164), NK1.1 (HB191) and then BioMag goat anti-mouse IgG, IgM, and goat anti-rat IgG (Qiagen). After negative magnetic enrichment, CD4⁺ cells were used either directly or further sorted into specific subpopulations. For sorting, cells were stained with antibodies to CD4 (RM4-5) and CD73 (TY/23), and in some experiments CD25 (PC61), and then isolated utilizing a FACSAria (BD Biosciences). Post-sort purity was routinely >99%. For adoptive transfer, total CD4⁺ or sorted T cells were washed and resuspended in sterile PBS. Recipient mice received ≦2.5×10⁶ cells i.v. in 200 μl of sterile PBS.

### Example 4

### Flow Cytometry

Cell suspensions were stained with fluorochrome-conjugated antibodies for CD4 (RM4-5), CD73 (TY/23), or FoxP3 (FJK-16s). Intracellular FoxP3 staining was carried out according to the manufacturer's instructions (eBioscience). Stained cells were acquired on a FACSCalibur (BD Biosciences). Data were analyzed with FlowJo software (Tree Star).

### Example 5

### T-Cell Cytokine Assay

Sorted T cells from MOG-immunized mice were cultured in the presence of irradiated C57BL/6 splenocytes with 0 or 10 μM MOG peptide. Supernatants were collected at 18 hrs and analyzed utilizing the Bio-plex cytokine (Biorad) assay for IL-2, IL-4, IL-5, IL-10, IL-13, IL-17, IL-1β, and TNFα.

### Example 6

### Immunohistochemistry (“IHC”)

Anesthetized mice were perfused with PBS, and brains, spleens, and spinal cords were isolated and snap frozen in Tissue Tek-OCT medium. Five μm sections (brains in a sagittal orientation) were affixed to Supefrost/Plus slides (Fisher), fixed in acetone, and stored at −80° C. For immunostaining, slides were thawed and treated with 0.03% H₂O₂ in PBS to block endogenous peroxidase, blocked with Casein (Vector) in normal goat serum (Zymed), and then incubated with anti-CD45 (YW62.3), anti-CD4 (RM4-5), or anti-ICAM-1 (3E2). Slides were incubated with biotinylated goat anti-rat Ig (Jackson ImmunoResearch) and streptavidin-HRP (Zymed) and developed with an AEC (Red) substrate kit (Zymed) and a hematoxylin counterstain. Cover slips were mounted with Fluoromount-G and photographed under light (Zeiss).

### Example 7

### Real Time q-PCR

Using Trizol (Invitrogen), RNA was isolated from the Z310 choroid plexus cell line (Zheng et al., “Establishment and Characterization of an Immortalized Z310 Choroidal Epithelial Cell Line from Murine Choroid Plexus,” Brain Res. 958:371-380 (2002), which is hereby incorporated by reference in its entirety). cDNA was synthesized using a Reverse-iT kit (ABGene). Primers (available upon request) specific for ARs were used to determine gene expression levels and standardized to the GAPDH housekeeping gene levels using a SYBR-Green kit (ABGene) run on an ABI 7500 real time PCR system. Melt curve analyses were performed to measure the specificity for each qPCR product.

### Example 8

### Evaluation of the Role of CD73 in EAE

Due to the immunomodulatory and immunosuppressive properties of adenosine, the role of CD73 in EAE was evaluated. Based on a report of exacerbated EAE in A1 adenosine receptor (AR)-deficient mice (Tsutsui et al., “A1 Adenosine Receptor Upregulation and Activation Attenuates Neuroinflammation and Demyelination in a Model of Multiple Sclerosis,” J. Neurosci. 24:1521-1529 (2004), which is hereby incorporated by reference in its entirety), cd73^{-/-} mice that are unable to catalyze the production of extracellular adenosine were expected to experience severe EAE. Surprisingly, cd73^{-/-} mice were highly resistant to the induction of EAE. However, CD4⁺ T cells from cd73^{-/-} mice do possess the capacity to generate an immune response against CNS antigens and cause severe EAE when adoptively transferred into cd73^{+/+} T cell-deficient mice. CD73⁺CD4⁺ T cells from wild type mice also caused disease when transferred into cd73^{-/-} recipients, indicating that CD73 expression, either on lymphocytes or in the CNS, is required for lymphocyte entry into the brain during EAE. Since cd73^{+/+} mice were protected from EAE induction by the broad spectrum AR antagonist caffeine and the A2A AR specific antagonist SCH58261, this data indicates that the extracellular adenosine generated by CD73, and not CD73 itself, regulates the entry of lymphocytes into the CNS during EAE. These results are the first to demonstrate a role for CD73 and adenosine in regulating the development of EAE.

### Example 9

### Cd73^{-/-} Mice are Resistant to EAE Induction

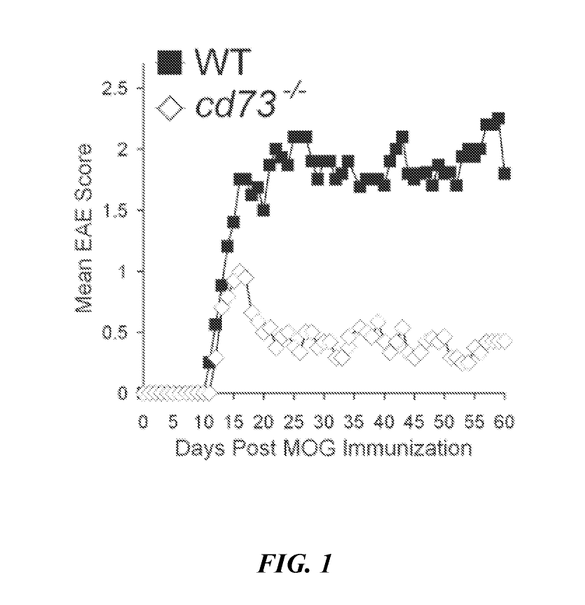

To determine if CD73 plays a role in controlling inflammation during EAE progression, cd73^{-/-} and wild type (cd73^{+/+}) mice were subjected to the myelin oligodendrocyte glycoprotein (“MOG”) EAE-inducing regimen (Swanborg, “Experimental Autoimmune Encephalomyelitis in Rodents as a Model for Human Demyelinating Disease,” Clin. Immunol. Immunopathol. 77:4-13 (1995); Bynoe et al., “Epicutaneous Immunization with Autoantigenic Peptides Induces T Suppressor Cells that Prevent Experimental Allergic Encephalomyelitis,” Immunity 19:317-328 (2003), which are hereby incorporated by reference in their entirety). Daily monitoring for EAE development revealed that cd73^{-/-} mice consistently displayed dramatically reduced disease severity compared to their wild type counterparts (FIG. 1). By three weeks after disease induction, cd73^{-/-} mice had an average EAE score of only 0.5 (weak tail) compared to 2.0 (limp tail and partial hind limb paralysis) for wild type mice (FIG. 1).

### Example 10

### CD4⁺ T Cells from Cd73^{-/-} Mice Respond to MOG Immunization

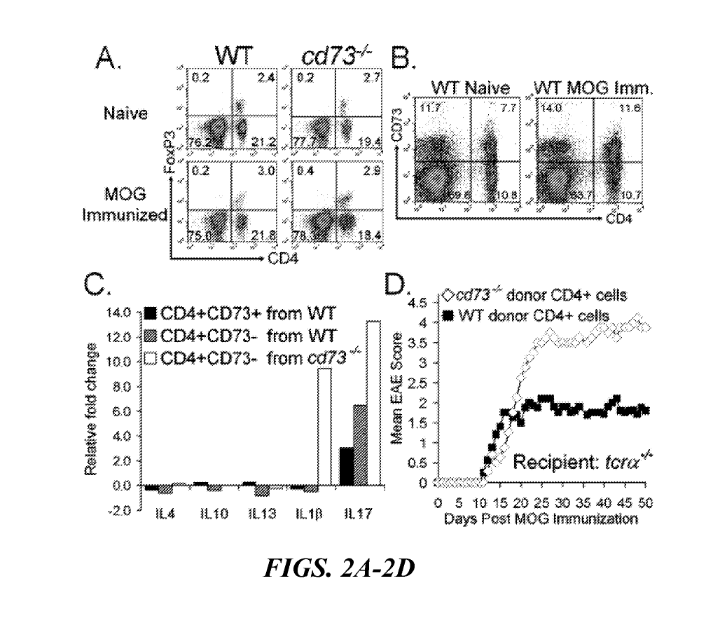

It was then asked whether the resistance of cd73^{-/-} mice to EAE induction could be explained by either an enhanced ability of cd73^{-/-} lymphocytes to suppress an immune response or an inability of these lymphocytes to respond to MOG stimulation. Naturally occurring CD4⁺CD25⁺FoxP3⁺ T cells, or Tregs, can regulate actively-induced EAE (Kohm et al., “Cutting Edge: CD4+CD25+ Regulatory T Cells Suppress Antigen-Specific Autoreactive Immune Responses and Central Nervous System Inflammation During Active Experimental Autoimmune Encephalomyelitis,” J. Immunol. 169:4712-4716 (2002), which is hereby incorporated by reference in its entirety). As Tregs have recently been shown to express CD73 and some reports suggest that the enzymatic activity of CD73 is needed for Treg function (Kobie et al., “T Regulatory and Primed Uncommitted CD4 T Cells Express CD73, Which Suppresses Effector CD4 T Cells by Converting 5′-Adenosine Monophosphate to Adenosine,” J. Immunol. 177:6780-6786); Deaglio et al., “Adenosine Generation Catalyzed by CD39 and CD73 Expressed on Regulatory T Cells Mediates Immune Suppression,” J. Exp. Med. 204:1257-1265 (2007), which are hereby incorporated by reference in their entirety), it was asked whether the number and suppressive activity of Tregs were normal in cd73^{-/-} mice. As shown in FIG. 2A, there were no significant differences in the frequencies of CD4⁺FoxP3⁺ Tregs in wild type and cd73^{-/-} mice, either before or after EAE induction. Similarly, the percentage of CD4⁺ T cells that expressed CD73 changed only modestly after EAE induction in wild type mice (FIG. 2B). Additionally, no significant difference was observed in the suppressive capacity of wild type and cd73^{-/-} Tregs to inhibit MOG antigen-specific CD4⁺ effector T cell proliferation. To determine whether cd73^{-/-} T cells can respond when stimulated with MOG peptide, the capacity of these cells to proliferate and produce cytokines was assessed. CD4⁺ T cells from MOG-immunized cd73^{-/-} and wild type mice displayed similar degrees of in vitro proliferation in response to varying concentrations of MOG peptide. However, CD4⁺ T cells from MOG-immunized cd73^{-/-} mice secreted higher levels of IL-17 and IL-1β following in vitro MOG stimulation, compared to those of wild type CD73⁺CD4⁺ or CD73⁻CD4⁺ T cells (FIG. 2C). Elevated levels of IL-17 are associated with MS (Matusevicius et al., “Interleukin-17 mRNA Expression in Blood and CSF Mononuclear Cells is Augmented in Multiple Sclerosis,” Mult. Scler. 5:101-104 (1999), which is hereby incorporated by reference in its entirety) and EAE development (Komiyama et al., “IL-17 Plays an Important Role in the Development of Experimental Autoimmune Encephalomyelitis,” J. Immunol. 177:566-573 (2006), which is hereby incorporated by reference in its entirety), while high levels of the proinflammatory IL-1β cytokine are a risk factor for MS (de Jong et al., “Production of IL-1beta and IL-1Ra as Risk Factors for Susceptibility and Progression of Relapse-Onset Multiple Sclerosis,” J. Neuroimmunol. 126:172-179 (2002), which is hereby incorporated by reference in its entirety) and an enhancer of IL-17 production (Sutton et al., “A Crucial Role for Interleukin (IL)-1 in the Induction of IL-17-Producing T Cells That Mediate Autoimmune Encephalomyelitis,” J. Exp. Med. 203:1685-1691 (2006), which is hereby incorporated by reference in its entirety). No difference in IL-2, IL-4, IL-5, IL-10, IL-13, INF-γ and TNF-α secretion was observed between wild type and cd73^{-/-} T cells following MOG stimulation (FIG. 2C). Overall, the results from these assays indicate that cd73^{-/-} T cells can respond well to MOG immunization.

It was then determined whether T cells from cd73^{-/-} mice possess the ability to cause EAE. To test this, CD4⁺ T cells from the spleen and lymph nodes of MOG immunized cd73^{-/-} mice were evaluated for their ability to induce EAE after transfer into tcrα^{-/-} (cd73^{+/+}) recipient mice. Tcrα^{-/-} mice lack endogenous T cells and cannot develop EAE on their own (Elliott et al., “Mice Lacking Alpha Beta+T Cells are Resistant to the Induction of Experimental Autoimmune Encephalomyelitis,” J. Neuroimmunol. 70:139-144 (1996), which is hereby incorporated by reference in its entirety). Cd73^{+/+}tcrα^{-/-} recipient mice that received CD4⁺ T cells from cd73^{-/-} donors developed markedly more severe disease compared to those that received wild type CD4⁺ T cells (FIG. 2D). Wild type and cd73^{-/-} donor CD4⁺ T cells displayed equal degrees of expansion following transfer into cd73^{+/+}tcrα^{-/-} recipient mice. Thus, CD4⁺ T cells from cd73^{-/-} mice are not only capable of inducing EAE, but cause more severe EAE than those derived from wild type mice when transferred into cd73^{+/+}tcrα^{-/-} mice. These results are consistent with in vitro assays in which cd73^{-/-} CD4⁺ T cells secreted elevated levels of IL-17 and IL-1β (which are known to exacerbate EAE) in response to MOG stimulation (FIG. 2C) and indicate that cd73^{-/-} mice are resistant to MOG-induced EAE because of a lack of CD73 expression in non-hematopoietic cells (most likely lack of expression in the CNS).

### Example 11

### Cd73^{-/-} Mice Exhibit Little/No Lymphocyte Infiltration into the CNS Following EAE Induction

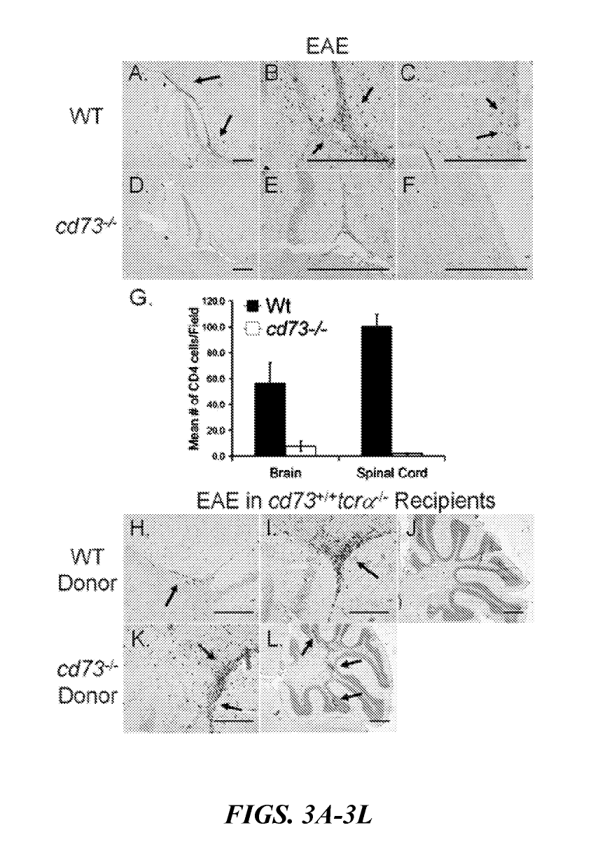

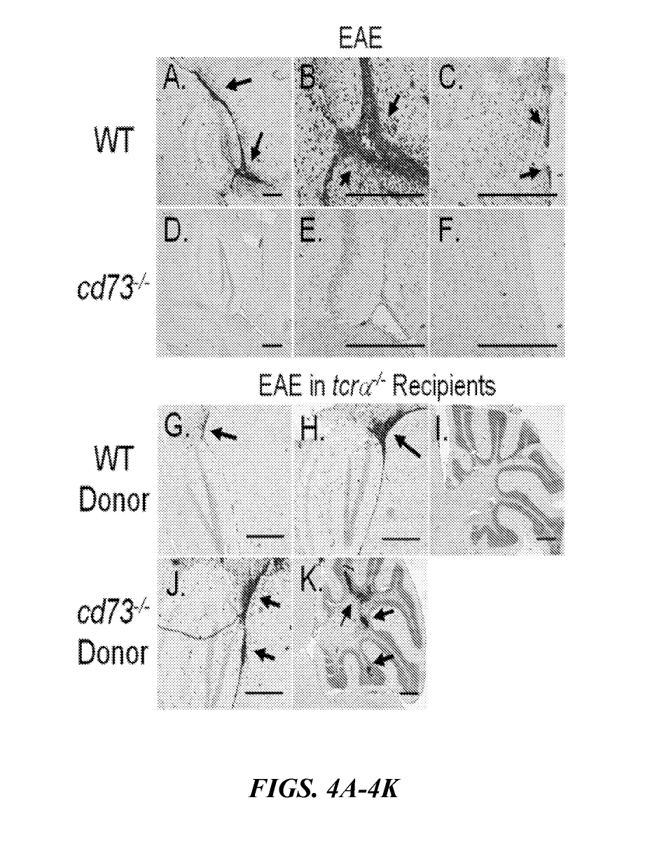

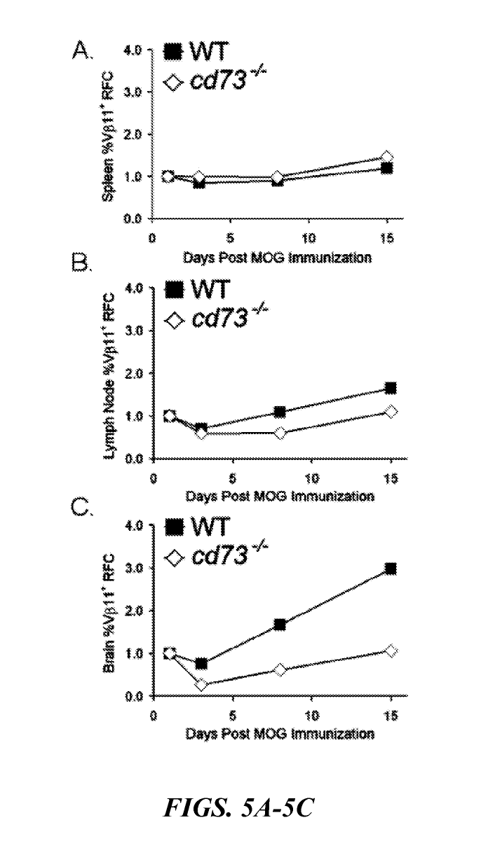

EAE is primarily a CD4⁺ T cell mediated disease (Montero et al., “Regulation of Experimental Autoimmune Encephalomyelitis by CD4+, CD25+ and CD8+ T Cells: Analysis Using Depleting Antibodies,” J. Autoimmun. 23:1-7 (2004), which is hereby incorporated by reference in its entirety) and during EAE progression, lymphocytes must first gain access into the CNS in order to mount their inflammatory response against CNS antigens, resulting in axonal demyelination and paralysis (Brown et al., “Time Course and Distribution of Inflammatory and Neurodegenerative Events Suggest Structural Bases for the Pathogenesis of Experimental Autoimmune Encephalomyelitis,” J. Comp. Neurol. 502:236-260 (2007), which is hereby incorporated by reference in its entirety). To determine if CNS lymphocyte infiltration is observed following EAE induction in cd73^{-/-} mice, brain and spinal cord sections were examined for the presence of CD4⁺ T cells and CD45⁺ cells by immunohistochemistry. Cd73^{-/-} mice displayed a dramatically lower frequency of CD4⁺ (FIGS. 3D-G) and CD45⁺ (FIG. 4 [Suppl. FIG. 1]) lymphocytes in the brain and spinal cord compared to wild type mice (FIGS. 3A-C, G) at day 13 post MOG immunization. Additionally, in lymphocyte tracking experiments where MOG-specific T cells from 2d2 TCR transgenic mice (Bettelli et al., “Myelin Oligodendrocyte Glycoprotein-Specific T Cell Receptor Transgenic Mice Develop Spontaneous Autoimmune Optic Neuritis,” J. Exp. Med. 197:1073-1081 (2003), which is hereby incorporated by reference in its entirety) were transferred into either wild type or cd73^{-/-} mice with concomitant EAE induction, the percentage of 2d2 cells in the CNS increased several fold with time in wild type recipient mice, but not at all in cd73^{-/-} recipients (FIG. 5). Overall, these results indicate that the observed protection against EAE induction in cd73^{-/-} mice is associated with considerably reduced CNS lymphocyte infiltration. Nevertheless, CD4⁺ T cells from MOG-immunized cd73^{-/-} mice possessed the ability to gain access to the CNS when transferred into cd73^{+/+} tcrα^{-/-} mice that were concomitantly induced to develop EAE (FIGS. 3K and 3L). In fact, earlier and more extensive CNS CD4⁺ lymphocyte infiltration was observed in cd73^{+/+}tcrα^{-/-} mice that received cd73^{-/-} CD4⁺ T cells (FIGS. 3K,L) than in those that received wild type CD4⁺ T cells (FIGS. 3H-J). Therefore, these results demonstrate that donor T cells from cd73^{-/-} mice have the ability to infiltrate the CNS of cd73^{+/+} recipient mice.

### Example 12

### CD73 Must be Expressed Either on Lymphocytes or in the CNS for Efficient EAE Development

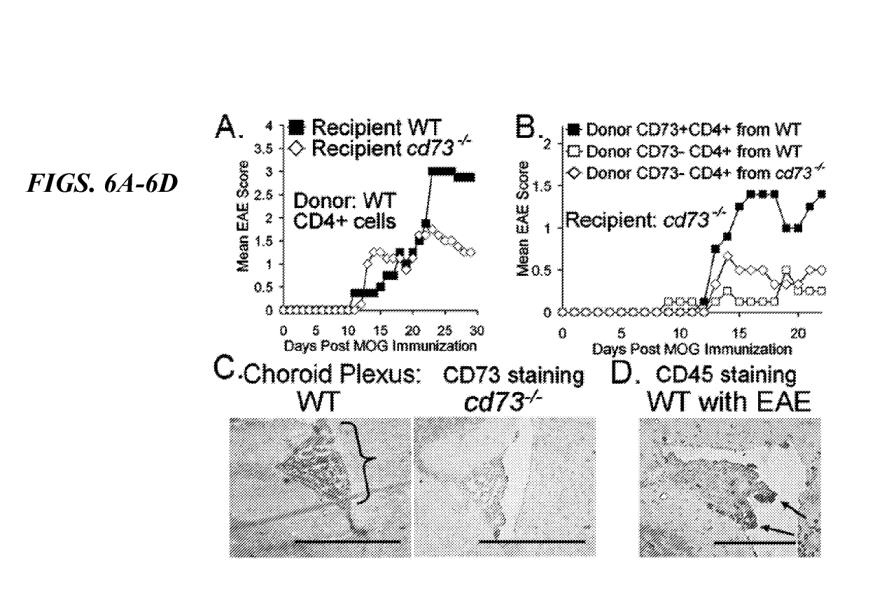

It was next asked whether CD73 expression on CD4⁺ T cells can compensate for a lack of CD73 expression in the CNS and allow the development of EAE. Therefore, CD4⁺ T cells were adoptively transferred from MOG-immunized wild type mice into cd73^{-/-} recipients, concomitantly induced EAE, and compared disease activity with that of similarly treated wild type recipients (FIG. 6A). While wild type recipients developed disease following EAE induction as expected, cd73^{-/-} recipients also developed prominent EAE with an average disease score of 1.5 by three weeks after disease induction. This was much higher than the 0.5 average score that cd73^{-/-} mice normally showed at this same time point (FIG. 1). To further define the association of CD4⁺ T cell CD73 expression with EAE susceptibility, sorted CD73⁺CD4⁺ and CD73⁻CD4⁺ T cells from immunized wild type mice, or total CD4⁺ (CD73⁻) T cells from immunized cd73^{-/-} mice, were transferred into cd73^{-/-} recipients with concomitant EAE induction (FIG. 6B). Cd73^{-/-} mice that received CD73⁺CD4⁺ T cells from wild type mice developed EAE with an average score of approximately 1.5 at three weeks post induction. Conversely, cd73^{-/-} mice that received wild type derived CD73⁻CD4⁺ T cells did not develop significant EAE. Additionally, CD4⁺ cells from cd73^{-/-} donor mice, which have the ability to cause severe EAE in CD73-expressing tcrα^{-/-} mice (FIG. 2D), were also incapable of potentiating EAE in recipient cd73^{-/-} mice (FIG. 6B). Therefore, although CD73 expression on T cells can partially compensate for a lack of CD73 expression in non-hematopoietic cells, EAE is most efficiently induced when CD73 is expressed in both compartments.

The identity of the CD73-expressing non-hematopoietic cells that promote the development of EAE is not known. Vascular endothelial cells at the BBB were considered as likely candidates, as many types of endothelial cells express CD73 (Yamashita et al., “CD73 Expression and Fyn-Dependent Signaling on Murine Lymphocytes,” Eur. J. Immunol. 28:2981-2990 (1998), which is hereby incorporated by reference in its entirety). However, immunohistochemistry revealed that mouse brain endothelial cells are CD73⁻. During these experiments, it was observed that CD73 is, however, highly expressed in the brain on the choroid plexus (FIG. 6C), which is an entry point into the CNS for lymphocytes during EAE progression (Brown et al., “Time Course and Distribution of Inflammatory and Neurodegenerative Events Suggest Structural Bases for the Pathogenesis of Experimental Autoimmune Encephalomyelitis,” J. Comp. Neurol. 502:236-260 (2007), which is hereby incorporated by reference in its entirety). FIG. 4D shows infiltrating lymphocytes in association with the choroid plexus of wild type mice 12 days post-EAE induction. Minimal CD73 staining was also observed on submeningeal regions of the spinal cord. Taken together, these results indicate that CD73 expression, whether on T cells or in the CNS (perhaps on the choroid plexus), is necessary for efficient EAE development.

### Example 13

### Adenosine Receptor Antagonists Protect Mice Against EAE Induction

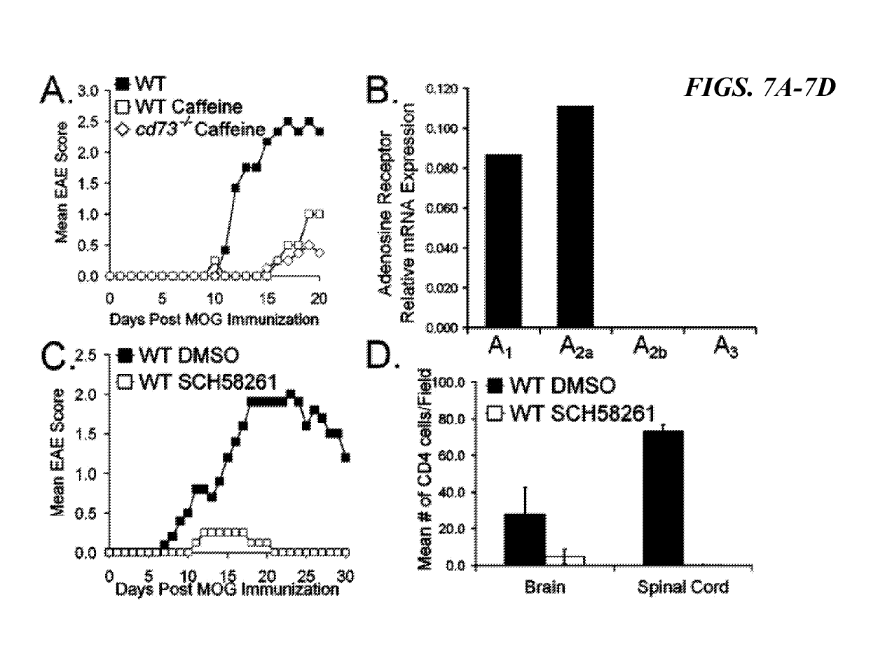

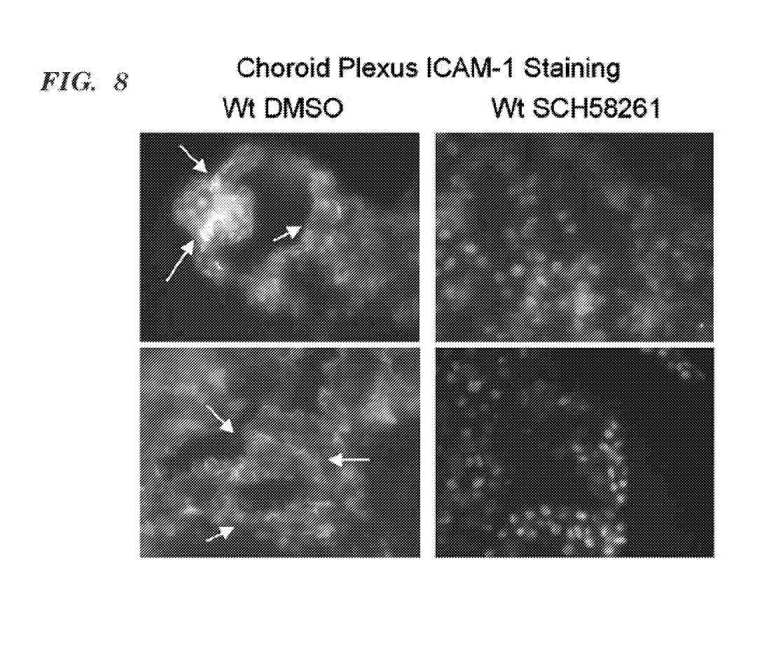

As CD73 catalyzes the breakdown of AMP to adenosine and ARs are expressed in the CNS (Tsutsui et al., “A1 Adenosine Receptor Upregulation and Activation Attenuates Neuroinflammation and Demyelination in a Model of Multiple Sclerosis,” J. Neurosci. 24:1521-1529 (2004)); Rosi et al., The Influence of Brain Inflammation Upon Neuronal Adenosine A2B Receptors,” J. Neurochem. 86:220-227 (2003), which are hereby incorporated by reference in their entirety), it was next determined if AR signaling is important during EAE progression. Wild type and cd73^{-/-} mice were treated with the broad spectrum AR antagonist caffeine (Dall'Igna et al., “Caffeine as a Neuroprotective Adenosine Receptor Antagonist,” Ann. Pharmacother. 38:717-718 (2004), which is hereby incorporated by reference in its entirety) at 0.6 g/L in their drinking water, which corresponds to an approximate dose of 4.0 mg/mouse of caffeine per day (Johansson et al., “A1 and A2A Adenosine Receptors and A1 mRNA in Mouse Brain: Effect of Long-Term Caffeine Treatment,” Brain Res. 762:153-164 (1997), which is hereby incorporated by reference in its entirety), 1 day prior to and throughout the duration of the EAE experiment (FIG. 7A). Wild type mice that received caffeine were dramatically protected against EAE development (FIG. 7A), comparable to previously published results (Tsutsui et al., “A1 Adenosine Receptor Upregulation and Activation Attenuates Neuroinflammation and Demyelination in a Model of Multiple Sclerosis,” J. Neurosci. 24:1521-1529 (2004), which is hereby incorporated by reference in its entirety). As expected, cd73^{-/-} mice that received caffeine did not develop EAE (FIG. 7A). Since CD73 is highly expressed on the choroid plexus (FIG. 6C), it was next determined if ARs are also expressed on the choroid plexus. Utilizing the Z310 murine choroid plexus cell line (Zheng et al., “Establishment and Characterization of an Immortalized Z310 Choroidal Epithelial Cell Line from Murine Choroid Plexus,” Brain Res. 958:371-380 (2002), which is hereby incorporated by reference in its entirety), only mRNA for the A1 and A2A adenosine receptor subtypes were detected by qPCR (FIG. 7B). As A1AR^{-/-} mice have been previously shown to develop severe EAE following disease induction (Tsutsui et al., “A1 Adenosine Receptor Upregulation and Activation Attenuates Neuroinflammation and Demyelination in a Model of Multiple Sclerosis,” J. Neurosci. 24:1521-1529 (2004), which is hereby incorporated by reference in its entirety), it was asked if treatment of wild type mice with SCH58261 (Melani et al., “The Selective A2A Receptor Antagonist SCH 58261 Protects From Neurological Deficit, Brain Damage and Activation of p38 MAPK in Rat Focal Cerebral Ischemia,” Brain Res. 1073-1074:470-480 (2006), which is hereby incorporated by reference in its entirety), an AR antagonist specific for the A2A subtype, could protect against EAE development. Wild type mice were given 1 mg/kg of SCH58261 in DMSO or DMSO alone both i.p. and s.c. (for a total of 2 mg/kg) 1 day prior to EAE induction and daily for 30 days throughout the course of the experiment (FIG. 7C). Wild type mice that received SCH58261 were dramatically protected against EAE development compared to wild type mice that received DMSO alone (FIG. 7C). Additionally, wild type mice given SCH58261 displayed a significantly lower frequency of CD4⁺ lymphocytes in the brain and spinal cord compared to DMSO treated wild type mice at day 15 post-EAE induction (FIG. 7D). As studies have shown that adhesion molecules (such as ICAM-1, VCAM-1, and MadCAM-1) on the choroid plexus play a role in the pathogenesis of EAE (Engelhardt et al., “Involvement of the Choroid Plexus in Central Nervous System Inflammation,” Microsc. Res. Tech. 52:112-129 (2001), which is hereby incorporated by reference in its entirety), it was determined if SCH58261 treatment affected adhesion molecule expression on the choroid plexus following EAE induction. Comparison of the choroid plexus from DMSO and SCH58261 treated wild type mice shows that A2A AR blockade prevented the up regulation of ICAM-1 that normally occurs during EAE progression (FIG. 8).

Based on these results, it was concluded that the inability of cd73^{-/-} mice to catalyze the generation of extracellular adenosine explains their failure to efficiently develop EAE following MOG immunization and that CD73 expression and A2A AR signaling at the choroid plexus are requirements for EAE progression.

The goal of this study was to evaluate the role of CD73 in EAE, an animal model for MS. As CD73 catalyzes the formation of extracellular adenosine which is usually immunosuppressive (Bours et al., “Adenosine 5′-Triphosphate and Adenosine as Endogenous Signaling Molecules in Immunity and Inflammation,” Pharmacol. Ther. 112:358-404 (2006), which is hereby incorporated by reference in its entirety) and A1AR^{-/-} mice exhibit severe EAE (Tsutsui et al., “A1 Adenosine Receptor Upregulation and Activation Attenuates Neuroinflammation and Demyelination in a Model of Multiple Sclerosis,” J. Neurosci. 24:1521-1529 (2004), which is hereby incorporated by reference in its entirety), applicants predicted that cd73^{-/-} mice would also develop severe EAE. However, cd73^{-/-} mice were highly resistant to EAE induction, a surprising finding considering the plethora of studies demonstrating that cd73^{-/-} mice are more prone to inflammation. For example, cd73^{-/-} mice are more susceptible to bleomycin-induced lung injury (Volmer et al., “Ecto-5′-Nucleotidase (CD73)-Mediated Adenosine Production is Tissue Protective in a Model of Bleomycin-Induced Lung Injury,” J. Immunol. 176:4449-4458 (2006), which is hereby incorporated by reference in its entirety) and are more prone to vascular inflammation and neointima formation (Zernecke et al., “CD73/ecto-5′-Nucleotidase Protects Against Vascular Inflammation and Neointima Formation,” Circulation 113:2120-2127 (2006), which is hereby incorporated by reference in its entirety). Consistent with these reports, applicants showed that cd73^{-/-} T cells produced higher levels of the EAE-associated proinflammatory cytokines IL-1β and IL-17 following MOG stimulation. Furthermore, the adoptive transfer of cd73^{-/-} T cells to tcrα^{-/-} mice, which lack T cells but express CD73 throughout their periphery, resulted in severe CNS inflammation following MOG immunization, consistent with a role for adenosine as an anti-inflammatory mediator. It is interesting to note that IFN-β treatment, one of the most effective therapies for MS, has been shown to up regulate CD73 expression on endothelial cells both in vitro and in vivo (Airas et al., “Mechanism of Action of IFN-Beta in the Treatment of Multiple Sclerosis: A Special Reference to CD73 and Adenosine,” Ann. N.Y. Acad. Sci. 1110:641-648 (2007), which is hereby incorporated by reference in its entirety). Thus, although the mechanism by which IFN-β benefits MS patients is incompletely understood, increased production of adenosine accompanied by its known anti-inflammatory and neuroprotective effects could be a factor.

Consistent with their resistance to EAE induction, cd73^{-/-} mice had a lower frequency of cells infiltrating the CNS during EAE compared to wild type mice. This was also an unexpected finding, as CD73-generated adenosine has previously been shown to restrict the migration of neutrophils across vascular endothelium during hypoxia and of lymphocytes across high endothelial venules of draining lymph nodes (Thompson et al., “Crucial Role for Ecto-5′-Nucleotidase (CD73) in Vascular Leakage During Hypoxia,” J. Exp. Med. 200:1395-1405 (2004), which is hereby incorporated by reference in its entirety). Applicants' data, in contrast, indicates that CD73, and the extracellular adenosine generated by CD73, are needed for the efficient passage of pathogenic T cells into the CNS. Therefore, the role that CD73 and adenosine play in CNS lymphocyte infiltration during EAE is more profound than their role in modulation of neuroinflammation.

It is important to know where CD73 must be expressed for T cell migration into the CNS. CD73 is found on subsets of T cells (Yamashita et al., “CD73 Expression and Fyn-Dependent Signaling on Murine Lymphocytes,” Eur. J. Immunol. 28:2981-2990 (1998), which is hereby incorporated by reference in its entirety) as well as on some epithelial (Strohmeier et al., “Surface Expression, Polarization, and Functional Significance of CD73 in Human Intestinal Epithelia,” J. Clin. Invest. 99:2588-2601 (1997), which is hereby incorporated by reference in its entirety) and endothelial cells (Yamashita et al., “CD73 Expression and Fyn-Dependent Signaling on Murine Lymphocytes,” Eur. J. Immunol. 28:2981-2990 (1998), which is hereby incorporated by reference in its entirety). The data presented here clearly demonstrates that although cd73^{-/-} T cells respond well to MOG immunization, they cannot enter the CNS unless CD73 is expressed in non-hematopoietic tissues (i.e. cd73^{+/+}tcrα^{-/-} mice which develop EAE after adoptive transfer of CD4⁺ T cells from cd73^{-/-} mice). A lack of CD73 on non-hematopoietic cells can also be compensated for, in part, by CD73 expression on T cells (i.e., cd73^{-/-} mice become susceptible to EAE when CD73⁺, but not CD73⁻, CD4⁺ T cells are adoptively transferred). While BBB endothelial cells as a relevant source of CD73 in the CNS were considered, because CD73 is expressed on human brain endothelial cells (Airas et al., “Mechanism of Action of IFN-Beta in the Treatment of Multiple Sclerosis: A Special Reference to CD73 and Adenosine,” Ann. N.Y. Acad. Sci. 1110:641-648 (2007), which is hereby incorporated by reference in its entirety), immunohistochemistry revealed that mouse brain endothelial cells are CD73⁻. However, CD73 was found to be highly expressed on choroid plexus epithelial cells, which form the barrier between the blood and the cerebrospinal fluid (CSF) and have a role in regulating lymphocyte immunosurveillance in the CNS (Steffen et al., “CAM-1, VCAM-1, and MAdCAM-1 are Expressed on Choroid Plexus Epithelium but Not Endothelium and Mediate Binding of Lymphocytes In Vitro,” Am. J. Pathol. 148:1819-1838 (1996), which is hereby incorporated by reference in its entirety). The choroid plexus has also been suggested to be the entry point for T cells during the initiation of EAE progression (Brown et al., “Time Course and Distribution of Inflammatory and Neurodegenerative Events Suggest Structural Bases for the Pathogenesis of Experimental Autoimmune Encephalomyelitis,” J. Comp. Neurol. 502:236-260 (2007), which is hereby incorporated by reference in its entirety). While the role of lymphocyte-brain endothelial cell interactions via VLA-4/VCAM-1 binding in both EAE and MS is well-documented (Rice et al., “Anti-Alpha4 Integrin Therapy for Multiple Sclerosis Mechanisms and Rationale,” Neurology 64:1336-1342 (2005), which is hereby incorporated by reference in its entirety), perhaps lymphocyte trafficking across the endothelial BBB is more important for disease maintenance and progression than for disease initiation, at least in EAE.

The next issue is how CD73 facilitates the migration of T cells into the CNS. Earlier work showed that lymphocyte CD73 can promote the binding of human lymphocytes to endothelial cells in an LFA-1-dependent fashion (Airas et al., “CD73 Engagement Promotes Lymphocyte Binding to Endothelial Cells Via a Lymphocyte Function-Associated Antigen-1-dependent Mechanism,” J. Immunol. 165:5411-5417 (2000), which is hereby incorporated by reference in its entirety). This does not appear to be the function of CD73 in EAE, however, because CD73-deficient T cells can enter the CNS and cause severe disease in cd73^{+/+}tcrα^{-/-} mice (FIG. 2D). Alternatively, CD73 can function as an enzyme to produce extracellular adenosine, a ligand for cell surface ARs. It is this latter function that is relevant for the current work given that AR blockade with caffeine or SCH58261 can protect mice from EAE. While the broad spectrum AR antagonist caffeine also inhibits certain phosphodiesterases (Choi et al., “Caffeine and Theophylline Analogues: Correlation of Behavioral Effects With Activity as Adenosine Receptor Antagonists and as Phosphodiesterase Inhibitors,” Life Sci. 43:387-398 (1988), which is hereby incorporated by reference in its entirety), its modulation of EAE progression is most likely through its effect on AR signaling (Tsutsui et al., “A1 Adenosine Receptor Upregulation and Activation Attenuates Neuroinflammation and Demyelination in a Model of Multiple Sclerosis,” J. Neurosci. 24:1521-1529 (2004), which is hereby incorporated by reference in its entirety). This notion is supported by the fact that SCH58261 also prevents EAE progression by specifically inhibiting A2A AR signaling. As CD73 and the A1 and A2A AR subtypes are expressed on the choroid plexus, extracellular adenosine produced by CD73 at the choroid plexus can signal in an autocrine fashion.

Adenosine signaling most likely regulates the expression of adhesion molecules at the choroid plexus. Studies have shown that the up regulation of the adhesion molecules ICAM-1, VCAM-1, and MadCAM-1 at the choroid plexus are associated with EAE progression (Engelhardt et al., Involvement of the Choroid Plexus in Central Nervous System Inflammation,” Microsc. Res. Tech. 52:112-129 (2001), which is hereby incorporated by reference in its entirety). As mice treated with the A2A AR antagonist SCH58261 do not experience increased choroid plexus ICAM-1 expression (FIG. 8), as normally occurs following EAE induction (Engelhardt et al., “Involvement of the Choroid Plexus in Central Nervous System Inflammation,” Microsc. Res. Tech. 52:112-129 (2001), which is hereby incorporated by reference in its entirety), the present results indicate that A2A AR signaling increases ICAM-1 during EAE progression.

In summary, this data shows that CD73 plays a critical role in the progression of EAE. Mice that lack CD73 are protected from the degenerative symptoms and CNS inflammation that are associated with EAE induction. This is the first study to demonstrate a requirement for CD73 expression and AR signaling for the efficient entry of lymphocytes into the CNS during EAE. The data presented here may mark the first steps of a journey that will lead to new therapies for MS and other neuroinflammatory diseases.

### Example 14

### The BBB Can be Modulated Through Activation of the Adenosine Receptors

The objective of this experiment was to determine if the blood brain barrier could be modulated by activation of adenosine receptors. NECA is a non-selective adenosine receptor agonist, with similar affinities for A1, A2A and A3 adenosine receptors and a low affinity for the A2b adenosine receptor. In order to determine if activation of adenosine receptors would induce extravasation of Evans Blue dye across the blood brain barrier (BBB), mice were treated with: NECA, a non-selective adenosine receptor agonist (n=5, 100 μl 0.01 nM); SCH58261, an A2A adenosine receptor specific antagonist (n=5, 1 mg/kg); pertussis toxin, an agent known to induce BBB leakiness and as such used as a positive control (n=7, 200 μl); and, PBS as a vehicle control (n=5, 100 μl). CD73^{-/-} mice, which lack the ability to produce extracellular adenosine, were also treated with NECA (n=4, 100 μl 0.01 nM). Treatments were administered as a single i.v. injection one hour prior to i.v. injection of 200 μl 1% Evans Blue dye (2 μg total dye injected). Four hours after administration of Evans Blue, mice were anesthetized with a ketamine/xylazine mix and perfused via the left ventricle with ice cold PBS. Brains were harvested and homogenized in n,n-dimethylformamide (DMF) at 5 μl/mg (v:w). Tissue was incubated for 72 hours at room temperature in DMF to extract the dye. Following extraction, the tissue/solvent mixture was centrifuged at 500×g for 30 minutes and 100 μl of supernatant was read on a BioTex spectrophotometer at 620 nm. Data is expressed as μg Evans Blue/ml DMF.

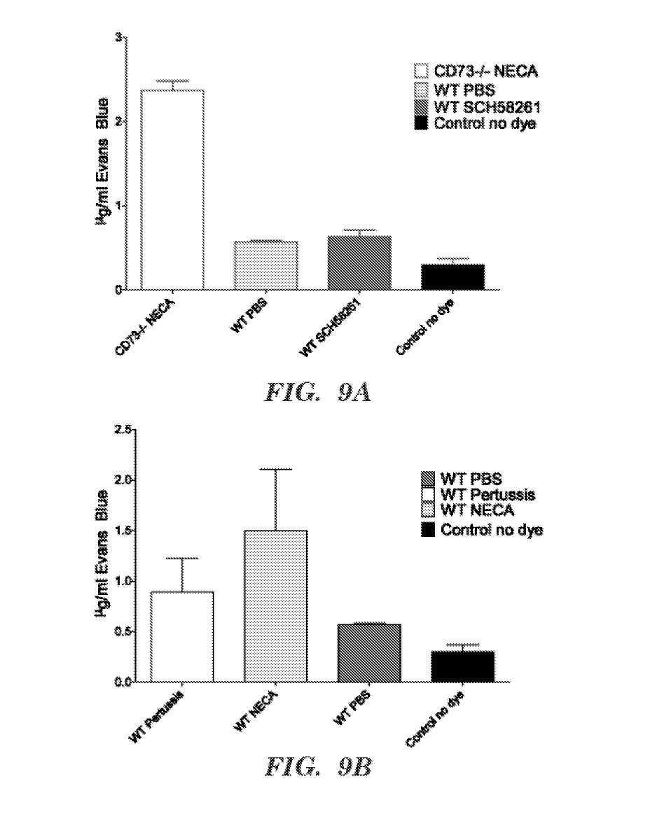

Treating mice with the general adenosine receptor agonist NECA can induce migration of dye across the blood brain barrier. This indicates that this barrier can be modulated through activation of the adenosine receptors. In FIG. 9A, CD73^{-/-} mice, which lack extracellular adenosine and thus cannot adequately signal through adenosine receptors, were treated with NECA, resulting in an almost five fold increase in dye migration vs. the PBS control. SCH58261 was used as a negative control since applicants have shown that blocking of the A2A adenosine receptor using this antagonist can prevent lymphocyte entry into the brain (Mills et al., “CD73 is Required for Efficient Entry of Lymphocytes into the Central Nervous System During Experimental Autoimmune Encephalomyelitis,” Proc. Natl. Acad. Sci. 105(27):9325-9330 (2008), which is hereby incorporated by reference in its entirety). In FIG. 9B, WT mice treated with NECA also show an increase over control mice. Pertussis is used as a positive control, as it is known to induce blood brain barrier leakiness in the mouse EAE model.

### Example 15

### The A2A and A2b Adenosine Receptors are Expressed on the Human Endothelial Cell Line hCMEC/D3

In order to establish an in vitro blood brain barrier (BBB), the human brain endothelial cell line hCMEC/D3 (Weksler et al., “Blood-brain Barrier-specific Properties of a Human Adult Brain Endothelial Cell Line,” J. Neurochem. 19(13):1872-4 (2005); Poller et al., “The Human Brain Endothelial Cell Line hCMEC/D3 as a Human Blood-brain Barrier Model for Drug Transport Studies,” J. Neurochem. 107(5):1358-1368 (2008), which are hereby incorporated by reference in their entirety) was obtained, which has been previously described as having BBB properties. Here, expression pattern of adenosine receptors on these cells was established.

hCMEC/D3 cells were grown to confluence, harvested and RNA was extracted using TRIzol reagent (Invitrogen, Carlsbad, Calif.) according to the manufacturer's instructions. cDNA was synthesized using a Verso cDNA kit (Thermo Scientific, Waltham, Mass.), and Real Time PCR was performed using Power SYBR Green (Applied Biosystems, Foster City, Calif.).

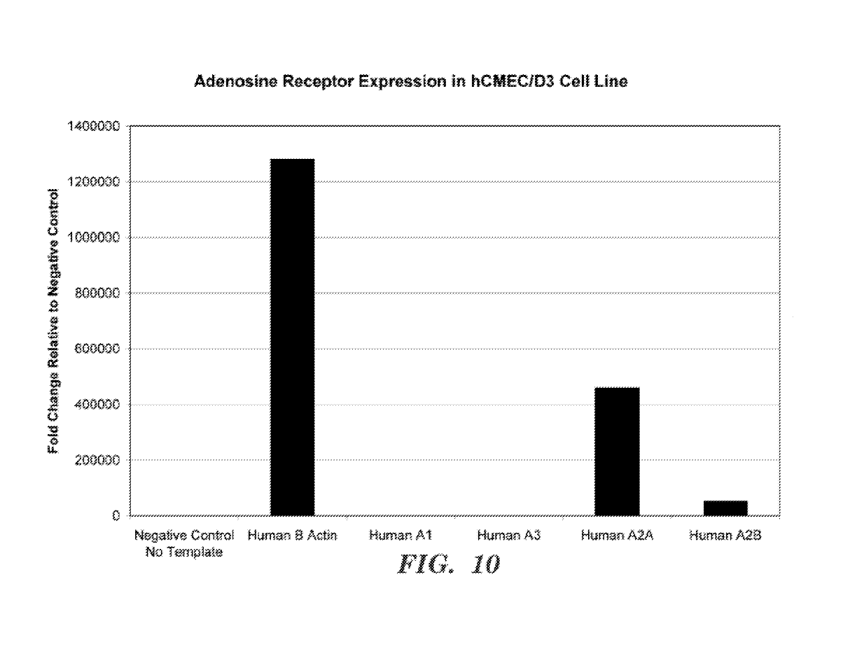

As shown in FIG. 10, the A2A and A2b adenosine receptors were found to be expressed on the human endothelial cell line hCMEC/D3.

### Example 16

### Adenosine Receptor Stimulation of Brain Endothelial Cells Promotes Lymphocyte Migration Through the BBB

The blood brain barrier (“BBB”) is comprised of endothelial cells. During late stages of EAE, lymphocytes are known to cross the BBB. In order to determine if adenosine receptor stimulation of brain endothelial cells could promote lymphocyte migration through the BBB, an in vitro BBB was established. The human brain endothelial cell line hCMEC/D3 (Weksler et al., “Blood-brain Barrier-specific Properties of a Human Adult Brain Endothelial Cell Line,” J. Neurochem. 19(13):1872-4 (2005); Poller et al., “The Human Brain Endothelial Cell Line hCMEC/D3 as a Human Blood-brain Barrier Model for Drug Transport Studies,” J. Neurochem. 107(5):1358-1368 (2008), which are hereby incorporated by reference in their entirety) was obtained, which has been previously described as having BBB properties.

hCMEC/D3 cells were seeded onto Transwell and allowed to grow to confluencey. 2×10⁶ Jurkat cells were added to the upper chamber with or without NECA (general adenosine receptor [AR] agonist), CCPA (A1 AR agonist), CGS 21860 (A2A AR agonist), or DMSO vehicle. After 24 hours, migrated cells in the lower chamber were counted. Values are relative to the number of cells that migrate through non-HCMECD3 seeded transwells.

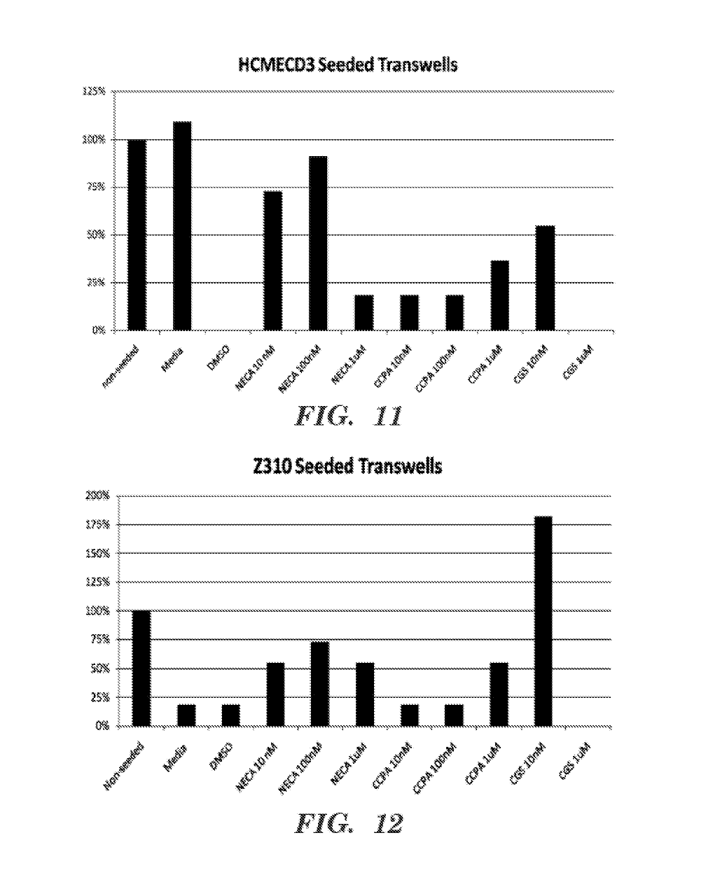

As shown in FIG. 11, NECA, a broad spectrum adenosine receptor agonist, induced some migration. CGS, the A2A adenosine receptor agonist, promoted lymphocyte migration across the in vitro BBB when used at a lower concentration. CCPA, the A1 agonist, induced lymphocyte migration at high levels possibly due to activation of the A2A adenosine receptor, which has a lower affinity for CCPA and thus is only activated at higher levels of CCPA.

### Example 17

### A2A Adenosine Receptor Activation Promotes Lymphocyte Migration Across the CP

The choroid plexus (“CP”) controls lymphocyte migration into the CNS. The CP expresses the A1 and A2A adenosine receptors. EAE is prevented in mice when A2A adenosine receptor activity is blocked. EAE is enhanced when the A1 adenosine receptor is missing. It was hypothesized that A2A adenosine receptor activation promotes lymphocyte migration across the CP. Z310 cells are a murine choroid plexus cell line.

To test the hypothesis, Transwell membranes were seeded with Z310 cells and allowed to grow to confluencey. 2×10⁶ Jurkat cells were added to the upper chamber with or with out NECA (n=1, general AR agonist), CCPA (n=1, A1 AR agonist), CGS 21860 (n=1, A2A AR agonist), or DMSO vehicle(n=1). After 24 hours, migrated cells in the lower chamber were counted. Values are relative to the number of cells that migrate through non-Z310 seeded transwells and the results are shown in FIG. 12.

As shown in FIG. 12, NECA, a broad spectrum adenosine receptor agonist, induced migration. CGS, the A2A adenosine receptor agonist, promoted lymphocyte migration across the CP. CCPA, the A1 agonist, induced lymphocyte migration at high levels possibly due to activation of the A2A adenosine receptor, which has a lower affinity for CCPA and as such is only activated at high levels of CCPA.

### Example 18

### Human Brain Endothelial Cells are Sensitive to Adenosine Receptor Induced cAMP Regulation

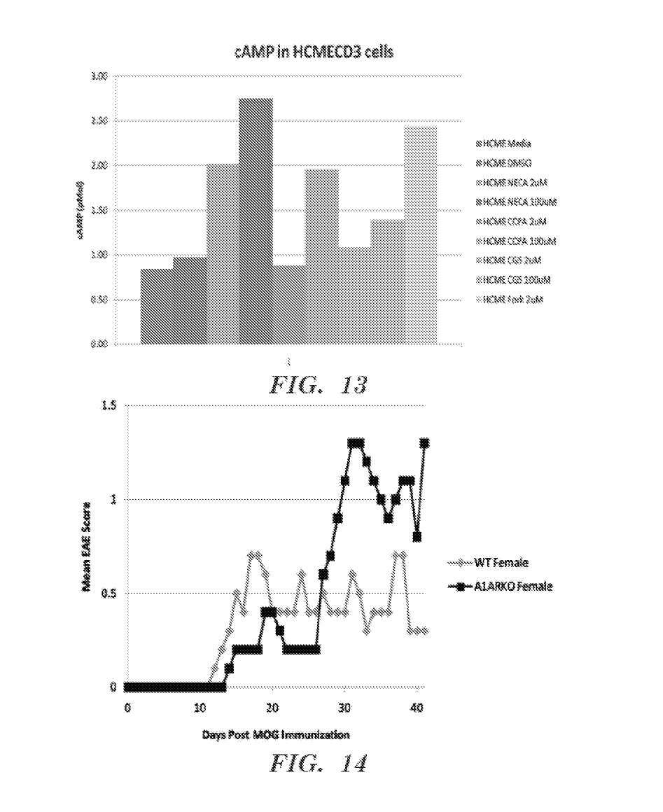

Adenosine receptor activation regulates cAMP levels in cells. In order to determine the sensitivity of human brain endothelial cells to adenosine receptor induced cAMP regulation, human brain endothelial cells were cultured with adenosine receptor agonists at various concentrations, followed by cAMP level analysis, as shown in FIG. 13.

HCMECD3 cells were grown to confluencey on 24 well plates. As adenosine receptor (“AR”) stimulation is known to influence cAMP levels, cells were treated with or without various concentrations of NECA (general AR agonist), CCPA (A1 AR agonist), CGS 21860 (A2A AR agonist), DMSO vehicle, or Forksolin (induces cAMP). After 15 minutes, lysis buffer was added and the cells were frozen at −80 C to stop the reaction. Duplicate samples were used for each condition. cAMP levels were assayed using a cAMP Screen kit (Applied Biosystems, Foster City, Calif.).

As shown in FIG. 13, the broad spectrum adenosine receptor agonist NECA increased cAMP levels, verifying that these cells can respond to adenosine receptor signaling. High levels of CCPA, the A1 adenosine receptor agonist, increased cAMP levels, again perhaps due to activation of the A2A adenosine receptor, which has a lower affinity for CCPA and as such is only activated at high levels of CCPA. CGS, the A2A adenosine receptor agonist slightly increased cAMP levels in the human brain endothelial cell line.

### Example 19

### Female A1 Adenosine Receptor Knockout Mice Develop More Severe EAE than Wild Type

A1 and A2A adenosine receptors are expressed on the choroid plexus. A2A adenosine receptor antagonists protect mice from EAE. Are mice that lack the A1 adenosine receptor prone to development of more severe EAE than wild type controls? To answer this question, disease profiles of wild type and A1 adenosine receptor null mice were compared.

Female A1 adenosine receptor knockout (A1ARKO, n=5) and wild type (WT, n=5) mice were immunized with CFA/MOG₃₅₋₅₅+PTX on Dec. 2, 2008 and scored daily for 41 days. As the results in FIG. 14 illustrate, A1ARKO mice develop more severe EAE than WT, and also develop disease at a faster rate than WT.

### Example 20

### Brains from Wild Type Mice Fed an Adenosine Receptor Antagonist have Higher Levels of FITC-Dextran than Brains from CD73^{-/-} Mice Fed an Adenosine Receptor Antagonist

In order to examine the effects of caffeine, a general adenosine receptor antagonist, on blood brain barrier permeability, mice were fed caffeine for several days and then injected with FITC Dextran, commonly used to assess endothelial permeability.

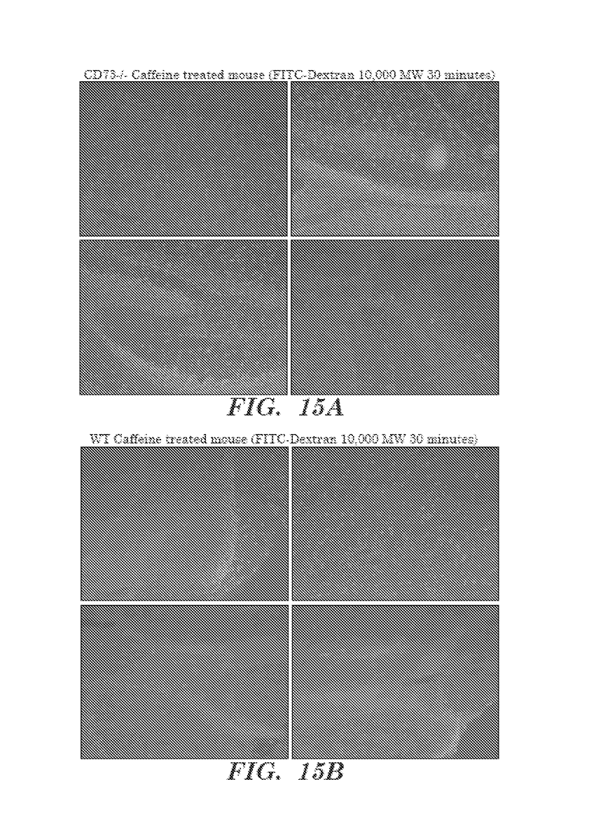

More particularly, mice were fed 0.6 g/l caffeine (Sigma, St. Louis, Mo.) in water or regular water ad lib for five days. Mice were injected IP with FITC Dextran (10,000 MW, Molecular Probes, Eugene, Oreg.) and after 30 minutes mice were perfused with ice cold PBS via the left ventricle. Brains were removed and snap frozen in OCT (Tissue Tek, Torrance, Calif.) and stored at −80° C. until sectioning. Tissue sections (5 μm) were stained with hematoxylin for light microscopy and with DAPI for a fluorescent counterstain. The results are shown in FIG. 15.

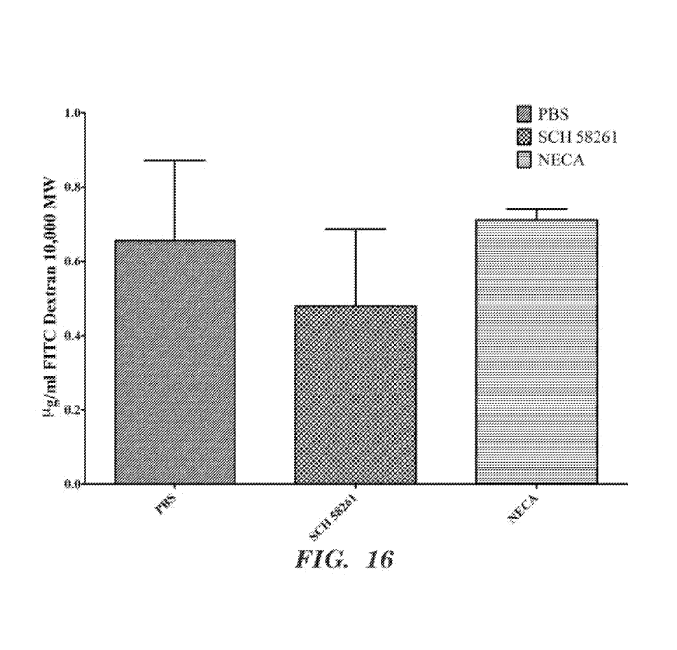

As shown in FIG. 15A, visualization of brain sections from CD73^{-/-} mice fed caffeine displayed a much less intense green color than wild type mice, indicating less FITC-Dextran extravasation across the blood brain barrier. Brain sections from wild type mice displayed an intensely green background (FIG. 15B) that is indicative of more FITC-dextran extravasation across the blood brain barrier. FIG. 16 shows the results for wild-type mice in graphical form.

### Example 21

### Adenosine Receptor Agonist NECA Increases Evans Blue Dye Extravasation Across the Blood Brain Barrier

The objective of this experiment was to determine if the blood brain barrier could be modulated by activation of adenosine receptors. NECA is a non-selective adenosine receptor agonist, with similar affinities for A1, A2A and A3 adenosine receptors and a low affinity for the A2B adenosine receptor.

In order to determine if activation of adenosine receptors would induce extravazation of Evans Blue dye across the blood brain barrier (BBB), mice were first treated on day one with NECA, a non-selective adenosine receptor agonist (n=2, 100 μl 0.01 nM); and, PBS as a vehicle control (n=2, 100 μl). On day 2 mice were then immunized with CFA-MOG₃₅₋₅₅ and pertussis to induce EAE. Then NECA or PBS was administered every other day on day 3, day 5, day 7 and day 9. On day 10, mice were injected intravenously with 200 μl 1% Evans Blue dye (2 μg total dye injected). Six hours after administration of Evans Blue, mice were anesthetized with a ketamine/xylazine mix and perfused via the left ventricle with ice cold PBS. Brains were harvested and homogenized in n,n-dimethylformamide (DMF) at 5 μl/mg (v:w). Tissue was incubated for 72 hours at room temperature in DMF to extract the dye. Following extraction, the tissue/solvent mixture was centrifuged at 500×g for 30 minutes and 100 μl of supernatant was read on a BioTex spectrophotometer at 620 nm. Data is expressed as pg Evans Blue/ml DMF and is shown in FIG. 17.

This experiment demonstrates that treatment of mice with the general adenosine receptor agonist NECA induces migration of Evans Blue dye into the CNS in mice immunized for EAE. This indicates that the blood brain barrier in the EAE model can be modulated through activation of the adenosine receptors. WT EAE mice treated with NECA show an increase in BBB permeability over PBS control EAE mice.

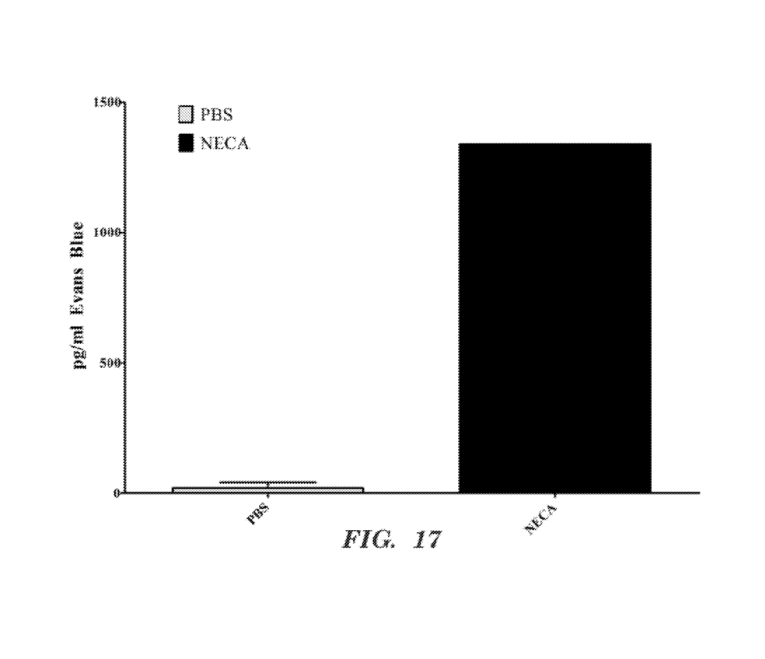

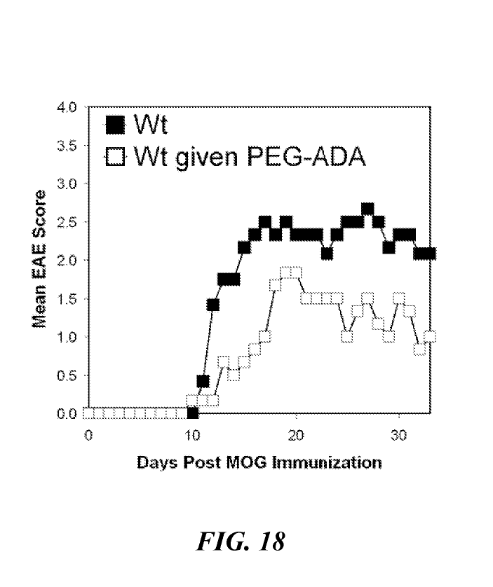

FIG. 18 shows the results in graphical form of an addition experiment that demonstrate PEGylated adenosine deaminase (“PEG-ADA”) treatment inhibits the development of EAE in wild-type mice. EAE was induced, disease activity was monitored daily, and mean EAE score was calculated in wild-type mice given either control PBS vehicle alone or 15 units/kg body weight of PEG-ADA i.p. every 4 days. Closed squares represent wild-type mice given PBS vehicle (n=3); open squares represent wild-type mice given PEG-ADA (n=3). These results demonstrate that adenosine deaminase treatment and adenosine receptor blockade protect wild type mice against EAE induction.

### Example 22

### Mouse and Rat Models

C57BL/6 mice from Jackson Laboratories were used as wild types. All mice used were aged 7-9 weeks and weighed between 20-25 g. All rats were female and aged 8 weeks and weighed 200-220 g. Mice and rats were bred and housed under specific pathogen-free conditions. All procedures were carried our in accordance with approved IACUC protocols.

### Example 23

### Administration of Drugs and Dextrans

The adenosine receptor agonists NECA, CCPA, CGS 21860, and SCH 58261 (Tocris, Ellisville, Mo.) were each dissolved in DMSO then diluted in PBS to the desired concentration; in most cases final DMSO concentrations were <0.5% (vol/vol). Lexiscan (Regadenoson; TRC, Inc., Toronto) was dissolved in PBS. For vehicle controls, DMSO was diluted in PBS to the same concentration. Dehydrated dextrans labeled with either FITC or Texas Red (Invitrogen, Carlsbad, Calif.) were re-suspended in PBS to 10 mg/ml. All experiments involving dextran injection used 1.0 mg dextran in PBS. In experiments where drug and dextran were injected concomitantly, 1.0 mg of dextran was mixed with the drug to the desired concentration in a final volume of 200 μl. In dose-response experiments, drugs and dextrans were injected concomitantly. All injections, except injections of SCH 58261, were retro-orbital i.v. with a 27-gauge needle. In the SCH 58261 experiment, SCH 58261 injections, 1 mg/kg, were intraperitoneal and mice were pre-dosed with this concentration daily for 4 days prior to the day of the experiment. An additional injection was administered at the time of the experiment. Vehicle/drug and dextrans were injected on day 5 and tissues were collected 3 h after vehicle/drug administration. In Lexiscan experiments, Lexiscan was administered i.v. with 3 injections, 5 min apart and tissues were collected at 15 min unless otherwise indicated.

### Example 24

### Treatment and Tissue Collection

In dose-response experiments and experiments with the A1 AR and A2A AR knock-out mice, drugs and dextrans were injected concomitantly. After 3 h, the mice were anesthetized with ketamine/xylazine and subjected to a nose cone containing isoflurane. They were perfused with 25-50 ml ice-cold PBS through the left ventricle of the heart then decapitated. Their brains were removed, weighed and frozen for later analysis.

### Example 25

### Fluorimetric Analysis of Dextrans in Brains

Ice-cold 50 mMTris-Cl (pH 7.6) was added to frozen brains (100 μl per 100 mg brain) and were to thawed on ice. They were homogenized with a dounce homogenizer and centrifuged at 16.1×g in a microfuge for 30 min at room temperature (rt). The supernatants were transferred to new tubes and an equal volume absolute methanol was added. The samples were centrifuged again at 16.1×g for 30 min at rt. Supernatant (200 μl) was transferred to a Corning costar 96 well black polystyrene assay plate (clear bottom). Additionally, a series of standards containing 0.001-10 pg/ml dextran in 50% Tris-Cl/50% absolute methanol (vol/vol) was added to each plate. Absolute concentrations of dextrans were derived from these standard curves. Fluorimetric analysis was performed on a BioTek (Winooski, Vt.) Synergy 4. FITC-dextran was detected at 488/519 (excitation/emission) and Texas Red-dextran was detected at 592/618.

### Example 26

### Primary Brain Endothelial Cell Isolation

This method has been adapted from previously described techniques. Song & Patcher, “Culture of murine brain microvascular endothelial cells that maintain expression and cytoskeletal association of tight junction-associated proteins,” In Vitro Cell. Dev. Biol. Anim. 39:313-320 (2003), which is hereby incorporated by reference in its entirety. Briefly, 12-week-old C57BL/6 mice were euthanized and decapitated. Dissected brains were freed from the cerebellum and large surface vessels were removed by carefully rolling the brains on sterile Whatman paper. The tissue was then homogenized in a Dounce homogenizer in ice-cold DMEM-F 12 medium, supplemented with L-glutamine and Pen/Strep, and the resulting homogenate was centrifuged at 3800×g, 4° C. for 5 min. After discarding the supernatant, the pellet was resuspended in 18% (w/vol) dextran in PBS solution, vigorously mixed, and centrifuged at 10000×g, 4° C. for 10 min. The foamy myelin layer was carefully removed with the dextran supernatant by gentle aspiration. The pellet was resuspended in pre-warmed (37° C.) digestion medium (DMEM supplemented with 1 mg/ml collagenase/dispase, 40 μg/ml DNaseI, and 0.147 μg/ml of the protease inhibitor tosyllysinechloromethylketone) and incubated at 37° C. for 75 min with occasional agitation. The suspension was centrifuged at 3800×g. The supernatant was discarded; the pellet was resuspended in pre-warmed (37° C.) PBS and centrifuged at 3800×g. The pellet was suspended in full medium (DMEM-F12 medium containing 10% plasma-derived serum, L-glutamine, 1% antibiotic-antimycotic, 100 mg/ml heparin, and 100 mg/ml endothelial cell growth supplement). The resulting capillary fragments were plated onto tissue culture dishes coated with murine collagen IV (50 μg/ml) at a density corresponding to one brainper 9.5 cm². Medium was exchanged after 24 h and 48 h. Puromycin (8 μg/ml) was added to the medium for the first two days. Before analysis, the primary mouse brain endothelial cells were grown until the culture reached complete confluence after 5-7 days in vitro.

### Example 27

### Cell Culture and qRT-PCR

The bEnd.3 mouse brain endothelial cell line was obtained from the ATCC (Manassas, Va.) and grown in ATCC formulated DMEM supplemented with 10% FBS. Using Trizol (Invitrogen) extraction, RNA was isolated from bEnd.3 cells. cDNA was synthesized using Multiscribe reverse transcriptase (Applied Biosystems, Carlesbad, Calif.). Primers (available upon request) specific for adenosine receptors and CD73 were used to determine gene expression levels and standardized to the TBP housekeeping gene levels using KapaSybr Fast (KapaBiosystems, Woburn, Mass.) run on a BioRad CFX96 real time qPCR system. Melt curve analyses were performed to measure the specificity for each qPCR product.

### Example 28

### Adenosine Receptor Western Blotting and Immunofluorescent Analysis

Primary mouse brain endothelial cells and Bend.3 cell cultures were grown as described above. Cells were lysed with 1 ml of lysis buffer containing protease inhibitor and condensed with TCA solution up to 200 μl. Samples were run on a 12% SDS-PAGE and transferred to Nitrocellulose paper. Membranes were blocked with 1% PVP (Polyvinyl Pyrrolidone) and incubated with anti A1 AR (AAR-006) and A2A AR (AAR-002) primary antibodies (Alomone Labs, Jerusalem, Israel) overnight. The membranes were washed and then incubated with goat-anti rabbit HRP. Membranes were washed thoroughly and developed with ECL solution and exposed to X-ray film. For adenosine receptor immunostaining, anesthetized mice were perfused with PBS and brains were isolated and snap frozen in Tissue Tek-OCT medium. Five μm sections (brains in a sagittal orientation) were affixed to Supefrost/Plus slides (Fisher), fixed in acetone, and stored at −80° C. Slides were thawed, washed in PBS, blocked with Casein (Vector) in normal goat serum (Zymed), and then incubated with anti-CD31 (MEC 13.3, BD Biosciences) and anti-A1 AR (A4104, Sigma) or Anti-A2A AR (AAR-002, Alomone Labs). Slides were then incubated with goat anti-rat Igalexafluor488 (Invitrogen) and goat anti-rabbit Ig Texas Red-X (Invitrogen). Sections were mounted with Vectashield mounting media with DAPI (Vector Laboratories, Burlingame, Calif.). Images were obtained on a Zeiss Axio Imager M1 fluorescent microscope.

### Example 29

### Fluorescence In Situ Hybridization (FISH)

For detection of adenosine receptor mRNA in brain endothelium, we performed FISH using FITC-labeled Cd31 and either Biotin-labeled A1 or A2A DNA oligonucleotide probes (Integrated DNA Technologies, probe sequences available upon request). Anesthetized mice were perfused with PBS and brains were isolated and snap frozen in Tissue Tek-OCT medium. Twelve micron cryosections were mounted on Superfrost/Plus slides (Fisher). After air drying on the slides for 30 minutes, the tissue was fixed in 4% neutral buffered paraformaldehyde (PFA) for 20 minutes and rinsed for 3 minutes in 1×PBS. Next, the tissue was equilibrated briefly in 0.1 M triethanolamine and acetylated for 10 minutes in 0.1 M triethanolamine with 0.25% acetic anhydride. Immediately following acetylation, the sections were dehydrated through an ascending ethanol series, and stored at room temperature. The tissue was rehydrated for 2×15 min in PBS, and equilibrated for 15 min in 5×SSC (NaCl 0.75M, Na-Citrate 0.075M). The sections were then prehybridized for 1 h at 42° C. in hybridization buffer (50% deionized formamide, 4×SSC, salmon sperm DNA 40 μg/ml, 20% (w/v) dextran sulphate, 1×Denhardt's solution). The probes (300 ng/ml) were denatured for 3 min at 80° C. and added to the pre-warmed (42° C.) buffer (hybridization mix). The hybridization reaction was carried out at 42° C. for 38 h with 250 μl of hybridization mix on each slide, covered with parafilm. Prehybridization and hybridization were performed in a black box saturated with a 4×SSC—50% formamide solution to avoid evaporation and photobleaching of FITC. After incubation, the sections were washed for 30 min in 2×SSC (room temperature), 15 min in 2×SSC (65° C.), 15 min in 0.2×SSC, 0.1% SDS (65° C.), and equilibrated for 5 min in PBS. For detection of the biotin-probes, sections were incubated for 30 min at room temperature with Texas-Red X conjugated streptavidine (Molecular Probes, S6370, 1 μg/ml) in PBS containing 1× Casein (Vector Laboratories). Excess streptavidin was removed by 15 min in PBS, followed by 15 min in 0.2×SSC, 0.1% SDS (65° C.), and 15 min in PBS washes. Sections were coverslipped with Vectashield mounting medium with DAPI (Vector Laboratories). Images were acquired using a Zeiss Axio Imager M1 fluorescent microscope.

### Example 30

### Injection and Anti-β-Amyloid Antibodies and Immunofluorescent Microscopy

Wild type and transgenic (AD) mice were given 0.80 μg NECA (i.v.). After 3 h, 400 μg of antibody to β-amyloid (200 μl of 2 mg/ml; clone 6E10, Covance, Princeton, N.J.) was administered i.v. and the mice rested for 90 min. Mice were then anesthetized and perfused as described above and their brains were placed in OTC and flash-frozen for later sectioning. Sagital sections (6 μm) were fixed in acetone for 10 min, then washed in PBS. Sections were blocked with casein for 20 min then incubated with 1:50 dilution of goat anti-mouse IgCy5 (polyclonal, 1 mg/ml, Abcam, Cabridge, Mass.) for 20 min then washed 3 times in PBS. Sections were then dried and mounted with VectashieldHardset mounting media with DAPI (Vector Laboratories, Burlingame, Calif.). Images were obtained on a Zeiss Axio Imager M1 fluorescent microscope.

### Example 31

### Transendothelial Cell Electrical Resistance (TEER) Assays

Bend.3 cells were grown in ATCC-formulated DMEM supplemented with 10% FBS on 24-well transwell inserts, 8 μm pore size (BD Falcon, Bedford, Mass.) until a monolayer was established. TEER was assessed using a Voltohmeter (EVOMX, World Precision Instruments, Sarasota, Fla.). Background resistance from un-seeded transwells was subtracted from recorded values to determine absolute TEER values. Change in absolute TEER from T0 for each individual transwell was expressed as percentage change and then averaged for each treatment group.

### Example 32

### F-Actin Staining of Endothelial Cells

Bend.3 cells were grown (as described above) on circular cover slips in 24-well plates. Cells were treated for 3 or 30 min with 1 μM CCPA, 1 μM Lexiscan, DMSO or media alone. Cover slips were washed with PBS, fixed in 4% paraformaldahyde, washed again in PBS and then permeabilized with 0.5% TritonX-100 in PBS. After washing in PBS/1% BSA, cover slips were blocked with 1% BSA then stained with Phallodin-Alexa 568. Cover slips were washed and mounted on slides with ProlongGold containing DAPI (Invitrogen). Images were obtained on an Olympus BX51 fluorescent microscope.

### Example 33

### Albumin Uptake Assay

Bend.3 cells grown on collagen coated coverslips were incubated with albumin-alexafluor 594 (50 mg/ml) (Invitrogen) and either media alone, DMSO vehicle, NECA (1 μM), or Lexiscan (1 μM) for 30 minutes. Albumin uptake was visualized (albumin=red) utilizing the Zeiss Axio Imager M1 fluorescent microscope. Total albumin fluorescence was recorded using Zeiss Axio Vision software, and measured utilizing Image-J software.

### Example 34

### Tight Junction Molecule Staining

Bend.3 cells grown on collagen coated coverslips were incubated with DMSO vehicle, NECA (1 μM), or Lexiscan (1 μM) for 1 h. Cells were washed with PBS, fixed with 4% paraformaldehyde, and permeabilized with 0.5% Triton-X in PBS. Cells were blocked with PBS/BSA/goat serum and then stained with antibodies (Invitrogen) against either ZO-1 (1A12), Claudin-5 (34-1600), or Occludin (3F10). Following a wash step, cells were incubated with either goat anti-rabbit IgTexas Red-X or goat anti-mouse IgCy5 (Invitrogen). Cover slips were washed and mounted on slides with ProlongGold containing DAPI. Images were obtained on a Zeiss Axio Imager M1 fluorescent microscope.

### Example 35

### Analysis Confirms that the Broad Spectrum AR Agonist NECA Increases BBB Permeability to Macromolecules

Statistical differences, assessed using the Students T-test, are indicated where P≦0.05.

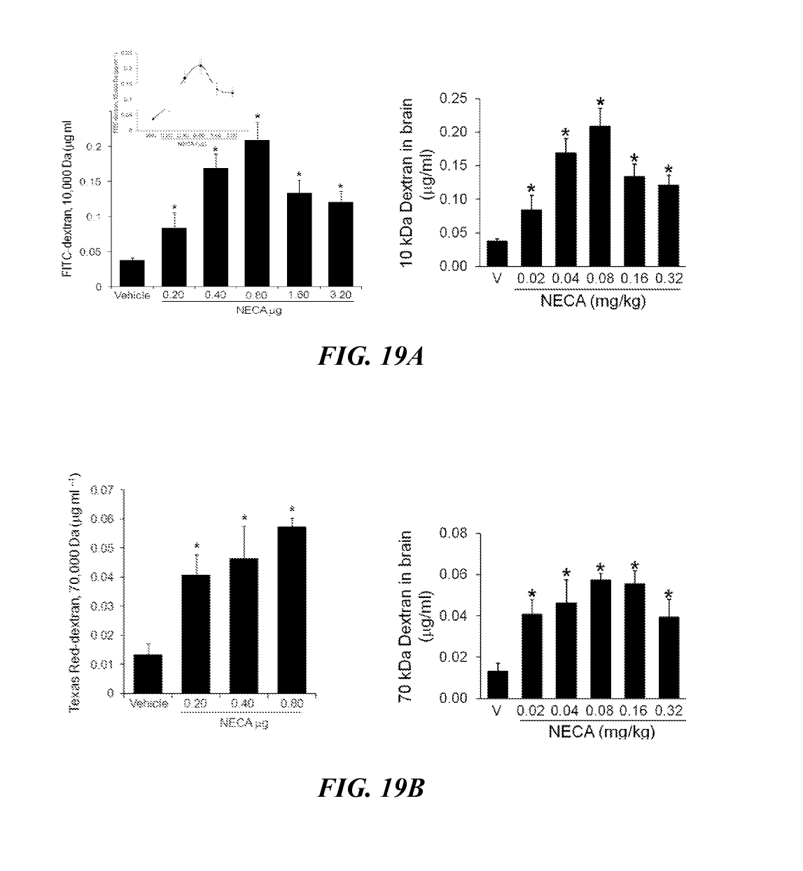

It was established that i.v. administration of NECA, which activates all ARs (A1, A2A, A2B, A3), resulted in a dose-dependent increase in extravasation of i.v.-administered fluorescently-labeled dextrans into the CNS of mice (FIG. 19). Importantly, it was observed that varying the dose of NECA resulted in dose-dependent increases in CNS entry of both 10,000 Da dextrans (FIG. 19A) and 70,000 Da dextrans (FIG. 19B) compared to treatment with vehicle alone. Maximum entry of dextrans into the CNS was observed with 0.08 mg/kg NECA. Higher concentrations of NECA had no additional effect or show diminished efficacy, possibly due to receptor desensitization (Ferguson et al., “Subtype-Specific Kinetics of Inhibitory Adenosine Receptor Internalization are Determined by Sensitivity to Phosphorylation by G Protein-coupled Receptor Kinases,” Mol. Pharmacol. 57:546-52 (2000), which is hereby incorporated by reference in its entirety). These results demonstrate that adenosine receptor activation increases BBB permeability.

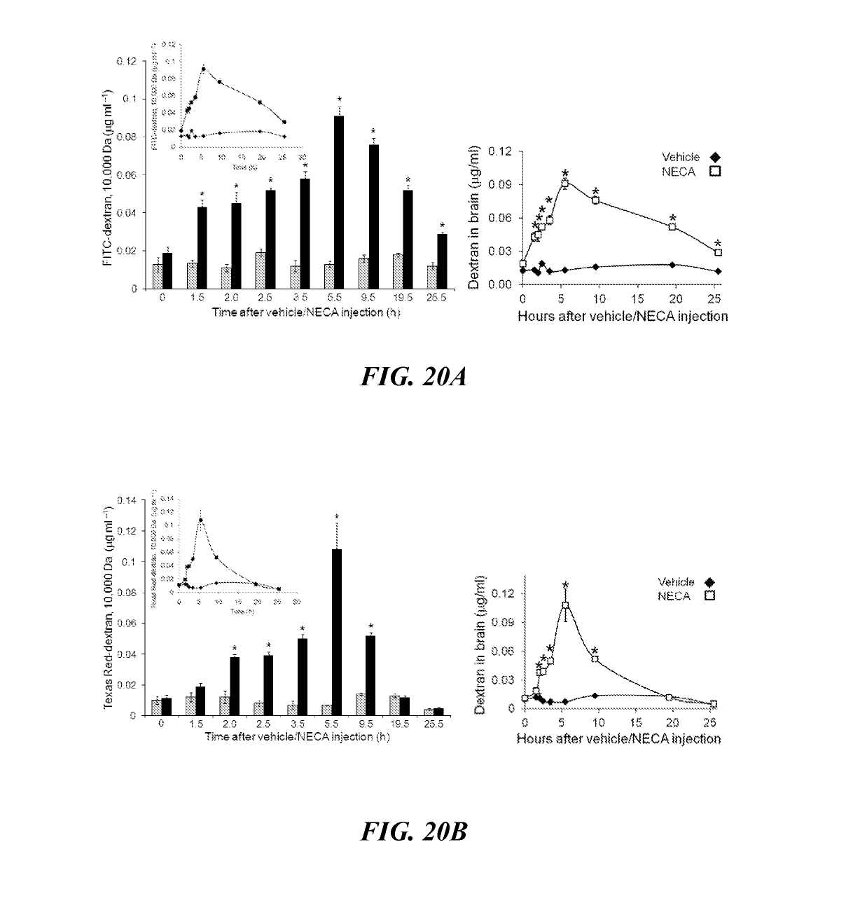

It was next determined the duration of BBB permeability after NECA administration and whether the process is reversible. In time-course experiments using the minimum effective dose of NECA determined by the dose-response experiments (0.08 mg/kg), it was observed that increased barrier permeability following NECA treatment is temporally discrete (FIG. 20A), with maximum entry of labeled dextran into the CNS observed between 4-6 h post-treatment. These data represent accumulation of FITC-dextran in the brain over time, since the dextran and NECA were administered at time zero (T₀). To determine how much dextran can enter the brain in a discrete period of time after NECA treatment, in a second experiment, dextran was administered at indicated times after NECA administration (FIG. 20B). These data represent dextran entry into the brain 90 min after dextran injection. At 8 h post-NECA treatment (9.5 h collection time), detectable levels of dextran in the brain were decreased from the maximum and by 18 h post-treatment (19.5 h collection time) the levels returned to baseline, as dextrans administered 18 h after NECA treatment were not detectable in the brain at significant levels (FIG. 20B). These results demonstrate that i.v. NECA administration results in a temporally discrete period of increased barrier permeability that returns to baseline by 8-18 h.

### Example 36

### A1 and A2A AR Activation Increases BBB Permeability

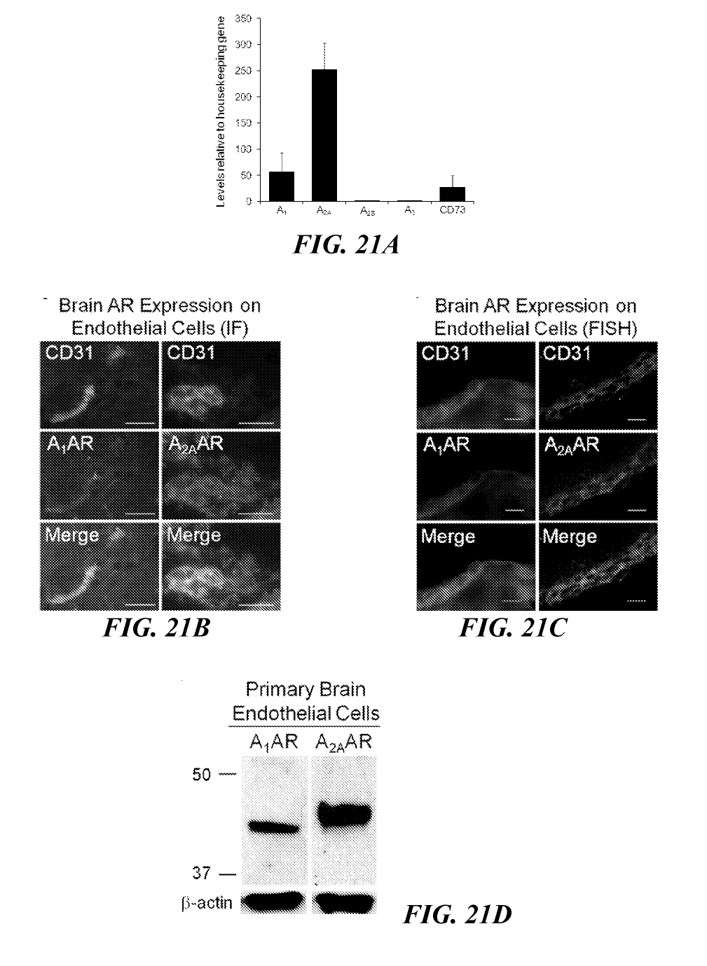

Four AR subtypes are expressed in mammals: A1, A2A, A2B, and A3 (Sebastiao et al., “Adenosine Receptors and the Central Nervous System,” Handb. Exp. Pharmacol. 471-534 (2009), which is hereby incorporated by reference in its entirety). To determine which ARs might function in barrier permeability, the levels of mRNA expression of each receptor subtype was examined in mouse brain endothelial cells. Expression of A1 and A2A receptors, but not A2B or A3 receptors, was detected in this cell line (FIG. 21A). Additionally, expression of CD73 and CD39, the two ecto-enzymes required for the catalysis of extracellular adenosine from ATP (CD39), was observed on cultured mouse brain endothelial cells. As AR activation increases BBB permeability to dextrans in mice, it was next determined if receptors for adenosine are expressed by mouse BECs. Utilizing antibodies and probes against the A1 and A2A ARs, expression of both ARs on CD31 co-stained endothelial cells within the brains of mice by immunofluorescent staining (FIG. 21B) and fluorescence in situ hybridization (FIG. 21C) was observed. Importantly, both A1 and A2A AR protein expression was detected by Western blot analysis on primary endothelial cells isolated from the brains of mice (FIG. 21D). Interestingly, the human brain endothelial cell line hCMEC/D3 also expresses both the A1 and A2A ARs. These data indicate that BECs are capable of directly responding to extracellular adenosine.

To investigate the functional contribution of A1 and A2A receptors in AR-mediated changes in BBB permeability, this effect was studied in mice lacking these receptors. Importantly, there were no significant differences in the basal levels of BBB permeability to 10,000 Da dextrans between WT, A₁ AR^{-/-} and A2A AR^{-/-} mice (FIGS. 21E, 21F, 21G). Following i.v. administration of NECA, both A₁^{-/-} and A2A^{-/-} mice showed significantly lower levels of i.v.-administered dextrans in their brains compared to wild type mice (FIGS. 21E and 21F). These data indicate that modulation of barrier permeability is, at least in part, mediated by these two AR subtypes. To examine the effect of NECA administration on BBB permeability in mice when neither the A1 nor the A2A AR is available for activation, A1 AR−/− mice were treated with the selective A2A antagonist SCH 58261 before NECA administration. When A2A AR signaling was blocked with this antagonist in mice lacking the A1 AR, no significant increase in BBB permeability was observed (FIG. 21G). These data indicate that modulation of BBB permeability is, at least in part, mediated by these two AR subtypes.

To confirm these results, the specific A1 agonist 2-chloro-N⁶-cyclopentyladenosine (CCPA) and the specific A2A agonist 4-[2-[[6-Amino-9-(N-ethyl-b-D-ribofuranuronamidosyl)-9H-purin-2yl]amino]ethyl]benzenepropanoic acid (CGS 21680) were administered to wild type mice. Both CGS 21680 (FIG. 21H) and CCPA (FIG. 21I) treatment resulted in increased dextran entry into the CNS and while this increase is substantial compared to vehicle treatment it was significantly lower than that observed after NECA administration. However, when used in combination, CCPA and CGS 21680 recapitulated the effect of increased dextran entry into the CNS that was observed with NECA treatment (FIG. 21J). These results confirmed that modulation of adenosine receptors facilitates entry of molecules into the CNS. Taken together, these results indicate that while activation of either the A1 or A2A AR on BECs can facilitateentry of molecules into the CNS, activation of both ARs has an additive effect.

### Example 37

### The Selective A2A AR Agonist Lexiscan Increases BBB Permeability

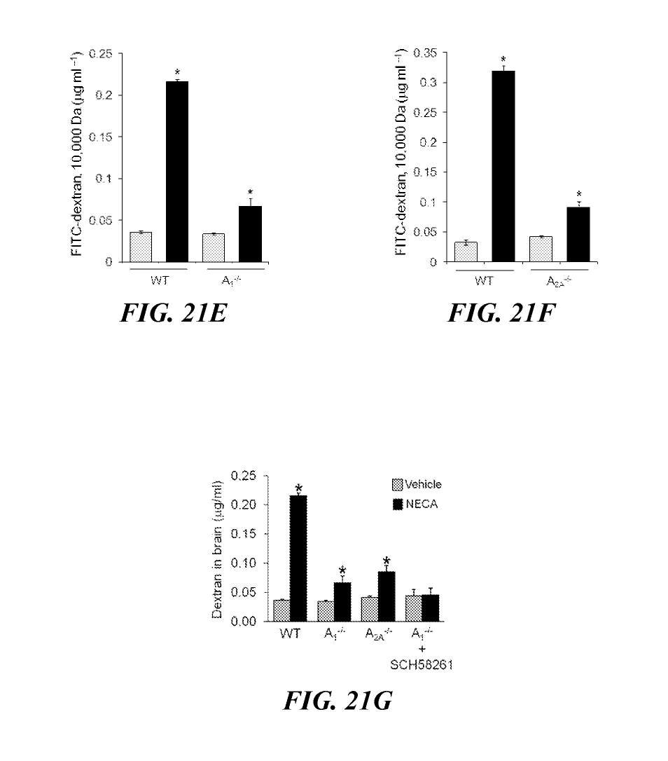

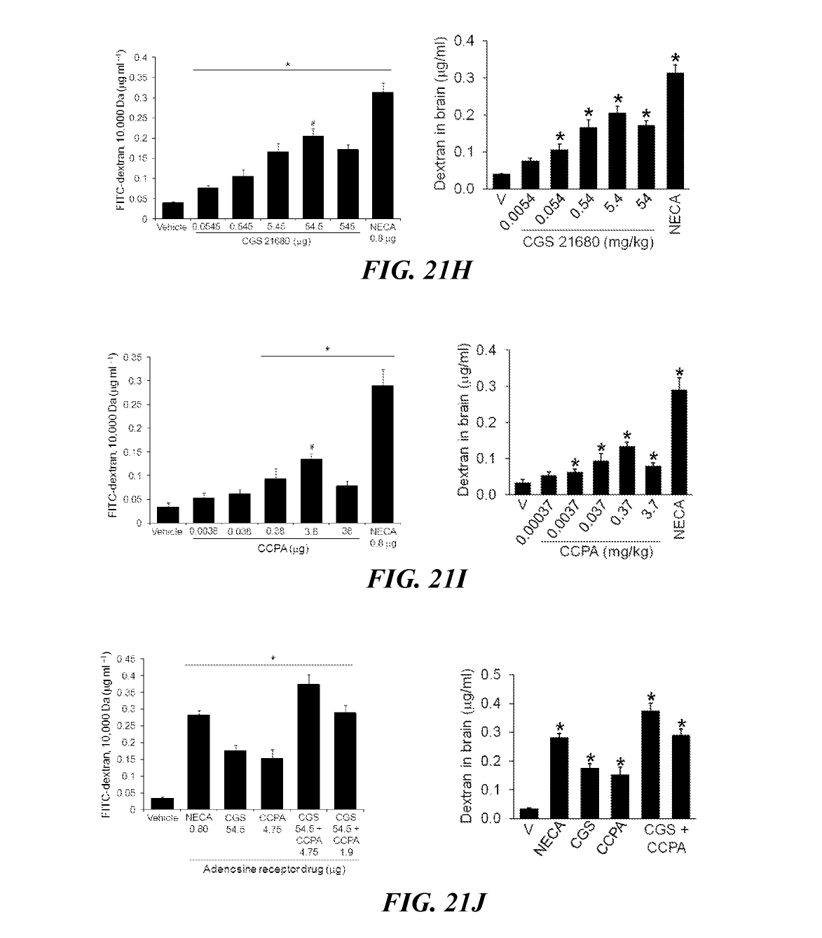

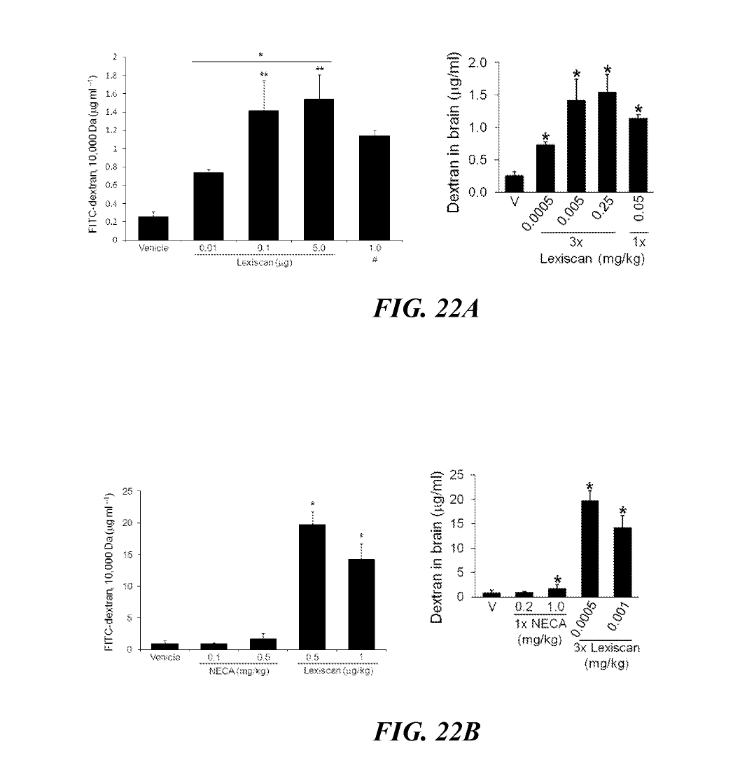

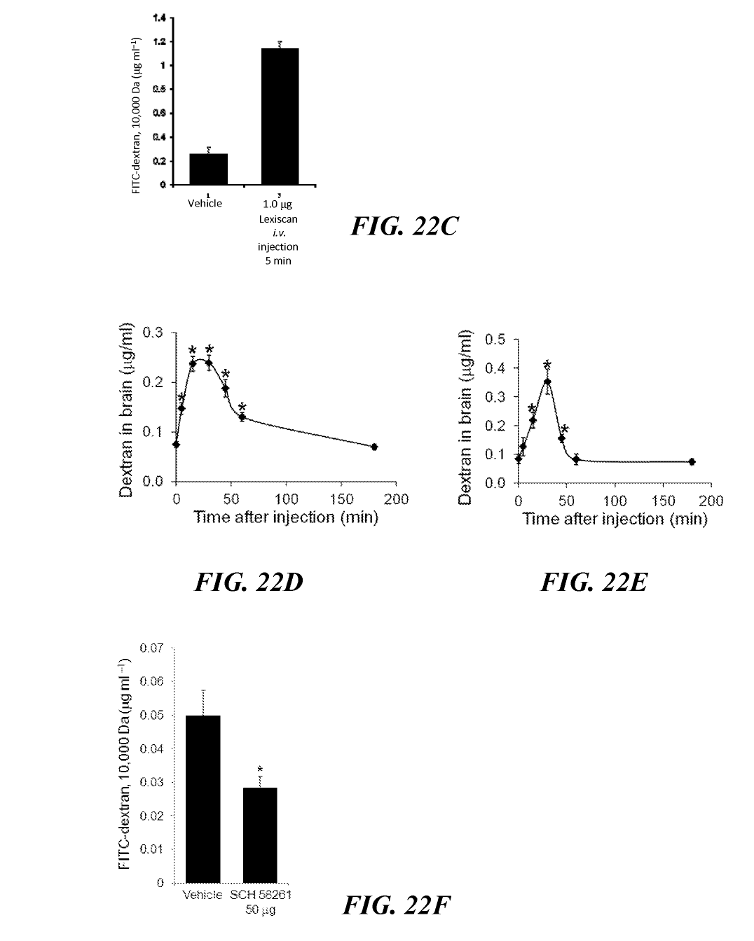

To explore the possible therapeutic use of AR agonism to facilitate CNS entry of i.v.-administered compounds, a commercially-available, FDA-approved AR agonist was tested in the experimental paradigm. The specific A2A AR agonist Lexiscan, which has been successfully used in myocardial perfusion imaging in humans (Iskandrian et al., “Adenosine Versus Regadenoson Comparative Evaluation in Myocardial Perfusion Imaging: Results of the ADVANCE Phase 3 Multicenter International Trial,” J. Nucl. Cardiol. 14:645-58 (2007), which is hereby incorporated by reference in its entirety), did indeed increase BBB permeability to 10,000 Da dextrans after i.v. administration (FIG. 22A) in mice. Interestingly, FITC-dextran was detectable in the brain after 5 min following a single Lexiscan injection. Additionally, i.v. administration of Lexiscan also increased BBB permeability in rats (FIG. 22B). The magnitude of increased BBB permeability after Lexiscan administration was much greater than the magnitude of increased permeability after NECA administration. Also, interestingly, the duration of increased BBB permeability correlates with the half-life of the AR agonist. For example, the time-course of BBB opening and closing after treatment with NECA (half-life ˜5 h) is much longer than the time-course after treatment with Lexiscan (half-life ˜3 min; (Astellas Pharma, “Lexiscan: U.S. Physicians Prescribing Information” (2009), which is hereby incorporated by reference in its entirety). In an injection paradigm intended to mimic continuous infusion of the drug, 3 injections of Lexiscan over 15 min resulted in dramatically high levels of labeled-dextran detected in the brains of rats (FIG. 22B). To examine the duration of Lexiscan's effects on BBB permeability, we determined CNS dextran entry over time in both mice and rats. FIG. 22C shows the results in graphical form of BBB permeability in rates to FITC-dextran administered simultaneously with 1 μg of Lexiscan at 5 minutes. Following a single i.v. injection of Lexiscan, maximum increased BBB permeability was observed after 30 min and returned to baseline by 180 min post-treatment (FIG. 22D). Similar results were observed after Lexiscan treatment in rats (FIG. 22E). Importantly, the duration of the effects on BBB permeability after Lexiscan treatment is much shorter than after NECA treatment, probably due to the different half-lives of the compounds (NECA ˜5 h, Lexiscan ˜3 min; (Astellas Pharma, “Lexiscan: U.S. Physicians Prescribing Information” (2009), which is hereby incorporated by reference in its entirety). The more than 20-fold increase in labeled-dextran in FIG. 22B (compared to single injections, FIG. 22E) is explained by a synergistic effect conferred on BBB opening as a result of multiple doses of Lexiscan.

These results demonstrate that in addition to the broad AR agonist, NECA, and the specific A1 and A2A AR agonists, CCPA and CGS 21680, used in this study, the FDA-approved A2A agonist Lexiscan increases BBB permeability to macromolecules.

### Example 38

### A2A Antagonism Decreases BBB Permeability

It was further hypothesized that if agonism of A1 and A2A receptors increases barrier permeability, then AR antagonism might decrease barrier permeability and prevent molecules from entering the CNS. It was previously observed that in WT mice, blockade of the A2A adenosine receptor inhibited leukocyte migration into the CNS (Mills et al., “CD73 is Required for Efficient Entry of Lymphocytes Into the Central Nervous System During Experimental Autoimmune Encephalomyelitis,” Proc Natl Acad Sci USA 105: 9325-30 (2008), which is hereby incorporated by reference in its entirety). This hypothesis was tested with a specific A2A AR antagonist. Intraperitoneal administration of the A2A AR antagonist 2-(2-Furanyl)-7-(2-phenylethyl)-7H-pyrazolo[4,3-e][1,2,4]triazolo[1,5-c]pyrimidin-5-amine (SCH 58261) resulted in significantly decreased entry of 10,000 Da FITC-dextran into WT mice brains (FIG. 22F). This data supports that blocking AR signaling tightens or closes the BBB.

### Example 39

### Antibodies to β-Amyloid Enter the Brain after NECA Administration

The most challenging therapeutic agents to get across the BBB are macromolecules such as antibodies, due to their enormous size (˜150 kDa). It was asked whether adenosine receptor modulation with NECA can facilitate the entry of antibodies into the CNS. To test this, a double [amyloid precursor protein (APP)/presenilin (PSEN)] transgenic mouse model of AD [strain B6.Cg-Tg(APPswe,PSEN1dE9)85Dbo/J] was used. These mice accumulate similar β-amyloid (Aβ) plaques that are a hallmark of AD (Jankowsky et al., “Mutant Presenilins Specifically Elevate the Levels of the 42 Residue Beta-amyloid Peptide in vivo: Evidence for Augmentation of a 42-specific Gamma Secretase,” Hum. Mol. Genet. 13:159-170 (2004); Mineur et al., “Genetic Mouse Models of Alzheimer's Disease,” Neural. Plast. 12:299-310 (2005), which are hereby incorporated by reference in their entirety).

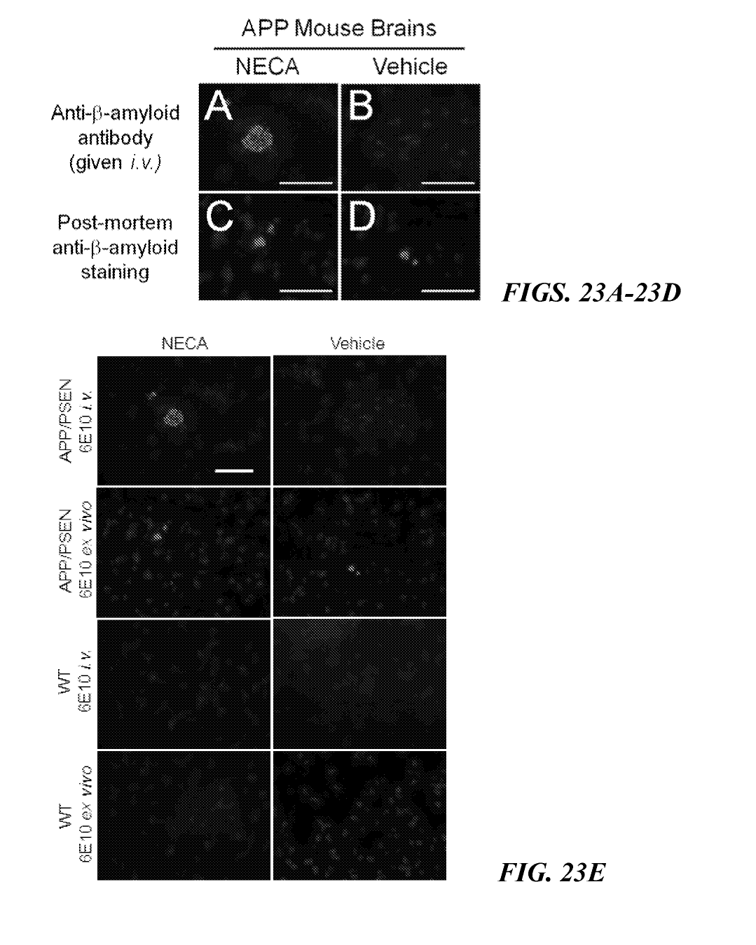

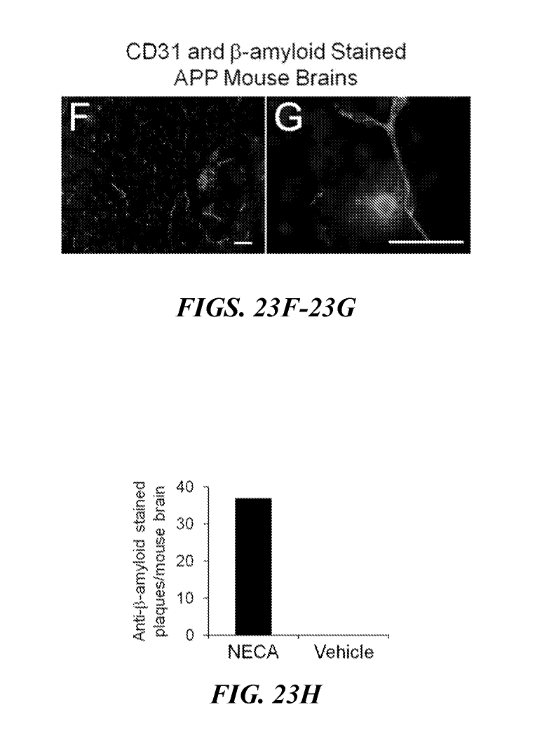

The monoclonal antibody 6E10 (Covance) has been shown to significantly reduce Aβ plaque burden in a mouse model of AD when administered by intracerebroventricular injection (Thakker et al., “Intracerebroventricular Amyloid-beta Antibodies Reduce Cerebral Amyloid Angiopathy and Associated Micro-hemorrhages in Aged Tg2576 Mice,” Proc. Natl. Acad. Sci. USA 106:4501-6 (2009), which is hereby incorporated by reference in its entirety). Three hours after i.v. NECA administration, the 6E10 antibody i.v. was administered. After 90 min, brains were collected, sectioned and stained with a secondary Cy5-labeled antibody. Binding of 6E10 antibody to Aβ plaques was observed throughout the brains of NECA-treated mice, with a concentration of Aβ plaques in the hippocampal region (FIGS. 23A and 23E). No binding of i.v. injected 6E10 antibody was observed in AD mice treated with vehicle alone (FIGS. 23A, 23B, and 23E) or in WT mice treated with NECA or vehicle. Neither NECA nor vehicle treatment alone affected the ability of AD mice to form Aβ plaques (FIGS. 23C and 23D). These results demonstrate that i.v. administered antibody to Aβ can cross the BBB following AR agonism and bind CNS Aβ plaques (FIG. 23H), most of which are located near blood vessels within the brain (FIGS. 23F and 23G). These results demonstrate that antibody to β-amyloid administered i.v. can cross the BBB after AR agonism.

### Example 40

### AR Activation Results in Decreased Transendothelial Resistance in Cultured Mouse BEC Monolayers

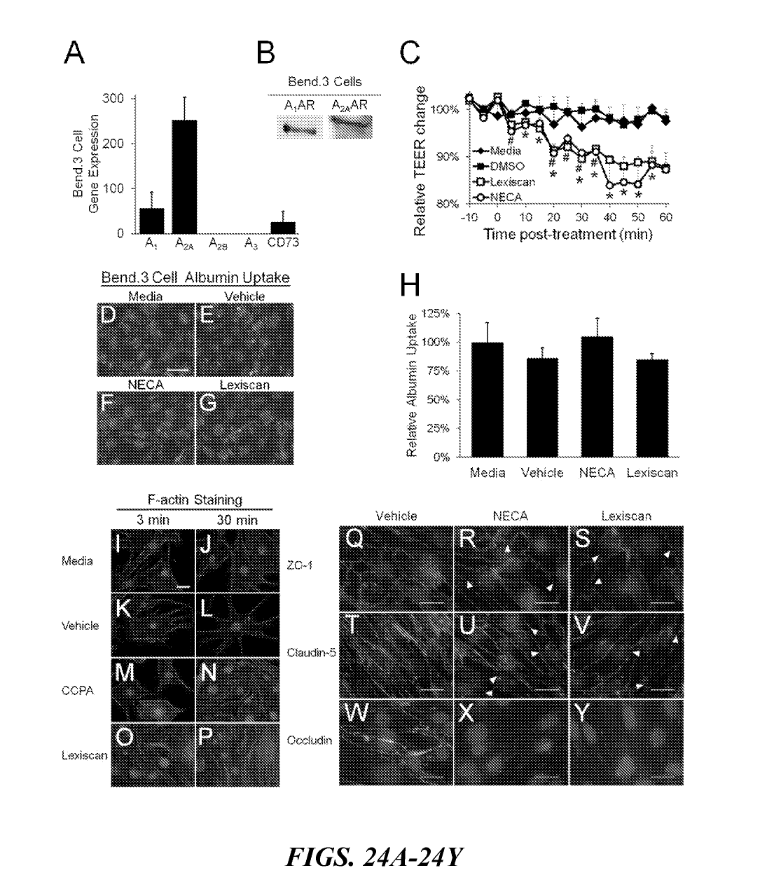

To determine how AR signaling mediates changes in BBB permeability, we utilized the pre-established mouse brain endothelial cell-line, Bend.3 (Montesano et al., “Increased Proteolytic Activity is Responsible for the Aberrant Morphogenetic Behavior of Endothelial Cells Expressing the Middle T Oncogene,” Cell 62:435-445 (1990), which is hereby incorporated by reference in its entirety). While there are four known AR subtypes expressed in mammals (A1, A2A, A2B and A3 (Sebastiao et al., “Adenosine Receptors and the Central Nervous System,” Handb. Exp. Pharmacol. 471-534 (2009), which is hereby incorporated by reference in its entirety), mRNA expression of the A1 and A2A receptors, but not A2B or A3 receptors, was detected in Bend.3 cells (FIG. 24A). Additionally, expression of CD73, anecto-enzyme required for the catalysis of extracellular adenosine from ATP, was observed on these cultured mouse BECs (FIG. 24A). Importantly, protein expression for the A1 and A2A ARs were detected on Bend.3 cells (FIG. 24B), indicating that these cells are capable of directly responding to extracellular adenosine.

Decreased transendothelial cell electrical resistance (TEER) in endothelial cell monolayers has been demonstrated to correlate with increased paracellular space between endothelial cells and increased barrier permeability (Wojciak-Stothard et al., “Rho and Rac But not Cdc42 Regulate Endothelial Cell Permeability,” J. Cell. Sci. 114:1343-1355 (2001); Dewi et al., “In vitro Assessment of Human Endothelial Cell Permeability: Effects of Inflammatory Cytokines and Dengue Virus Infection,” J. Virol. Methods 121:171-180 (2004), which are hereby incorporated by reference in their entirety). In transwell assays with monolayers of cultured mouse BECs (starting absolute TEER mean=182 ohms; median=187 ohms), we observed decreases in TEER after addition of NECA or Lexiscan, as compared with BECs given vehicle or media alone (FIG. 24C). While AR signaling alters TEER in BECs, we did not observe any alterations in the rate of transcytosis in BECs following AR stimulation. For instance, NECA and Lexiscan induced AR signaling did not affect the rate of fluorescently-labeled albumin uptake in BECs, as compared to media and vehicle treated controls (FIGS. 24D-24H).

### Example 41

### AR Activation Correlates with Actinomyosin Stress Fiber Formation and Alterations in Tight Junctions in Brain Endothelial Cells

The actin cytoskeleton is vital for the maintenance of cell shape and for endothelial barrier integrity. Since actomyosin stress fibers are necessary for inducing contraction in cell shape (Hotulainen et al., “Stress Fibers are Generated by Two Distinct Actin Assembly Mechanisms in Motile Cells,” J. Cell. Biol. 173:383-94 (2006); Prasain et al., “The Actin Cytoskeleton in Endothelial Cell Phenotypes,” Microvasc. Res. 77:53-63 (2009), which are hereby incorporated by reference in their entirety), it was hypothesized that adenosine receptor signaling results in actin stress fiber induction.

To test this, brain endothelial cells (“BECs”) were treated with either CCPA (to agonize A1 adenosine receptors) or Lexiscan (to agonize the A2A adenosine receptor) (FIGS. 24I-24P). The marked induction of actinomycin stress fibers was observed upon A1 and A2A agonist treatment as compared to treatment with vehicle alone, as shown in FIGS. 24I-24L. This indicates that activation of ARs induces changes in cytoskeletal elements in BECs to increase barrier permeability.

While AR signaling induces changes in TEER, which is a functional measure of paracellular permeability, and actinomyosin stress fibers, which regulate cell shape, it is important to determine if AR signaling alters the junctional interactions between BECs. Therefore to determine if AR signaling alters the tight junction of BECs, Bend.3 cells were cultured to confluent monolayers and determined if the expression of ZO-1, claudin-5, or occludin was altered following AR agonist treatment (FIGS. 24Q-24Y). While confluent Bend.3 cells formed proper tight junctions when grown in media or treated with vehicle (FIGS. 24Q, 24T, and 24W), AR agonist treatment induced alterations in tight junction protein expression. For example, Bend.3 cells treated with NECA or Lexiscan had severely diminished occludin expression following treatment with discreet alterations in ZO-1 and claudin-5 (FIGS. 24X and 24Y). Overall, these results indicate BEC permeability can be altered by AR signaling through changes tight junction molecule expression.

As shown schematically in FIG. 25, these results demonstrate that activation of either the A1 or A2A AR temporarily increases BBB permeability, while activation of both receptors results in an additive effect of increased BBB permeability. It is shown here that BBB permeability mediated through A1 and A2A ARs operates as a door where activation opens the door and local adenosine concentration is the key.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method for increasing blood brain barrier permeability in a subject, comprising administering to said subject an agent which activates both of A1 and A2A adenosine receptors.

2. The method according to claim 1, wherein the increase in blood brain permeability lasts up to 18 hours.

3. The method according to claim 1, wherein the agent which activates both of A₁ and A2A adenosine receptors is an agonist of both A1 and A2A receptors.

4. The method according to claim 3, wherein the agent which activates both of A1 and A2A adenosine receptors is a broad spectrum adenosine receptor agonist.

5. The method according to claim 3, wherein the agonist of both A1 and A2A receptors is AMP 579.

6. The method according to claim 4, wherein the agonist of both A1 and A2A receptors is NECA.

7. The method according to claim 3, wherein the activation of both A1 and A2A receptors is synergistic with respect to blood brain barrier permeability.

8. The method according to claim 3, wherein the activation of both A1 and A2A receptors is additive with respect to blood brain barrier permeability.

9. A method for increasing blood brain barrier permeability in a subject, comprising administering to said subject an A1 adenosine receptor agonist and an A2A adenosine receptor agonist.

10. The method according to claim 9, wherein the A1 adenosine receptor agonist and an A2A receptor agonist are A1-selective and A2-selective adenosine receptor agonists.

11. The method according to claim 9, wherein the A1 adenosine receptor agonist and an A2A receptor agonist are formulated in a single unit dosage form.

12. The method according to claim 9, wherein the A1 adenosine receptor agonist and an A2A receptor agonist are administered simultaneously.

13. The method according to claim 9, wherein the A1 adenosine receptor agonist and an A2A receptor agonist are administered sequentially.

14. The method according to claim 10, wherein the A1-selective adenosine receptor agonist is selected from the group consisting of CCPA, 8-cyclopentyl-1,3-dipropylxanthine, R-phenylisopropyl-adenosine, N6-Cyclopentyladenosine, N(6)-cyclohexyladenosine, and combinations thereof.

15. The method according to claim 10, wherein the A2A-selective adenosine receptor agonist is selected from the group consisting of Lexiscan, CGS 21680, ATL-146e, YT-146 (2-(1-octynyl)adenosine), DPMA (N6-(2-(3,5-dimethoxyphenyl)-2-(2-methylphenyl)ethyl)adenosine), and combinations thereof.

16. A composition comprising an A1 adenosine receptor agonist and an A2A adenosine receptor agonist, and a pharmaceutically acceptable carrier, excipient, or vehicle.

17. The composition according to claim 16, wherein the A1 adenosine receptor agonist and an A2A receptor agonist are A1-selective and A2-selective adenosine receptor agonists.

18. The composition according to claim 16, further comprising a therapeutic agent.

19. The composition according to claim 18, wherein the therapeutic agent is suitable for treating a CNS disease, disorder, or condition.

20. The composition according to claim 19, wherein the therapeutic agent is selected from the group consisting of acetaminophen, acetylsalicylic acid, acyltransferase, alprazolam, amantadine, amisulpride, amitriptyline, amphetamine-dextroamphetamine, amsacrine, antipsychotics, antivirals, apomorphine, arimoclomol, aripiprazole, asenapine, aspartoacyclase enzyme, atomoxetine, atypical antipsychotics, azathioprine, baclofen, beclamide, benserazide, benserazide-levodopa, benzodiazepines, benztropine, bevacizumab, bleomycin, brivaracetam, bromocriptine, buprenorphine, bupropion, cabergoline, carbamazepine, carbatrol, carbidopa, carbidopa-levodopa, carboplatin, chlorambucil, chlorpromazine, chlorprothixene, cisplatin, citalopram, clobazam, clomipramine, clonazepam, clozapine, codeine, COX-2 inhibitors, cyclophosphamide, dactinomycin, dexmethylphenidate, dextroamphetaine, diamorphine, diastat, diazepam, diclofenac, donepezil, doxorubicin, droperidol, entacapone, epirubicin, escitalopram, ethosuximide, etoposide, felbamate, fluoxetine, flupenthixol, fluphenazine, fosphenytoin, gabapentin, galantamine, gamma hydroxybutyrate, gefitinib, haloperidol, hydantoins, hydrocordone, hydroxyzine, ibuprofen, ifosfamide, IGF-1, iloperidone, imatinib, imipramine, interferons, irinotecan, KNS-760704, lacosamide, lamotrigine, levetiracetam, levodopa, levomepromazine, lisdexamfetamine, lisuride, lithium carbonate, lypolytic enzyme, mechlorethamine, mGluR2 agonists, memantine, meperidine, mercaptopurine, mesoridazine, mesuximide, methamphetamine, methylphenidate, minocycline, modafinil, morphine, N-acetylcysteine, naproxen, nelfinavir, neurotrin, nitrazepam, NSAIDs, olanzapine, opiates, oseltamivir, oxaplatin, paliperidone, pantothenate kinase 2, Parkin, paroxetine, pergolide, periciazine, perphenazine, phenacemide, phenelzine, phenobarbitol, phenturide, phenyloin, pimozide, Pink1, piribedil, podophyllotoxin, pramipexole, pregabalin, primidone, prochlorperazine, promazine, promethazine, protriptyline, pyrimidinediones, quetiapine, rasagiline, remacemide, riluzole, risperidone, ritonavir, rituximab, rivastigmine, ropinirole, rotigotine, rufinamide, selective serotonin reuptake inhibitors (SSRIs), selegine, selegiline, sertindole, sertraline, sodium valproate, stiripentol, taxanes, temazepam, temozolomide, tenofovir, tetrabenazine, thiamine, thioridazine, thiothixene, tiagabine, tolcapone, topiramate, topotecan, tramadol, tranylcypromine, trastuzumab, tricyclic antidepressants, trifluoperazine, triflupromazine, trihexyphenidyl, trileptal, valaciclovir, valnoctamide, valproamide, valproic acid, venlafaxine, vesicular stomatitis virus, vigabatrin, vinca alkaloids, zanamivir, ziprasidone, zonisamide, zotepine, zuclopenthixol, and combinations thereof.

21. A method for delivering a macromolecular therapeutic agent to the brain of a subject, comprising administering to said subject: (a) an agent which activates both of A1 and A2A adenosine receptors; and (b) the macromolecular therapeutic agent.

22. The method according to claim 21, wherein the agent which activates both of A1 and A2A adenosine receptors is administered before the macromolecular therapeutic agent.

23. The method according to claim 21, wherein the agent which activates both of A1 and A2A adenosine receptors is administered simultaneously with the macromolecular therapeutic agent.

24. The method according to claim 21, wherein the agent which activates both of A1 and A2A adenosine receptors is administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the macromolecular therapeutic agent is administered.

25. The method according to claim 21, wherein the macromolecular therapeutic agent is a monoclonal antibody.

26. The method according to claim 25, wherein the macromolecular therapeutic agent is a monoclonal antibody selected from the group consisting of 6E10, PF-04360365, 131I-chTNT-1/B MAb, 131I-L19SIP, 177Lu-J591, ABT-874, AIN457, alemtuzumab, anti-PDGFR alpha monoclonal antibody IMC-3G3, astatine At 211 monoclonal antibody 81C6, Bapineuzumab, Bevacizumab, cetuximab, cixutumumab, Daclizumab, Hu MiK-beta-1, HuMax-EGFr, iodine I 131 monoclonal antibody 3F8, iodine I 131 monoclonal antibody 81C6, iodine I 131 monoclonal antibody 8H9, iodine I 131 monoclonal antibody TNT-1/B, LMB-7 immunotoxin, MAb-425, MGAWN1, Me1-14 F(ab′)2, M-T412, Natalizumab, Neuradiab, Nimotuzumab, Ofatumumab, Panitumumab, Ramucirumab, ranibizumab, SDZ MSL-109, Solanezumab, Trastuzumab, Ustekinumab, Zalutumumab, Tanezumab, Aflibercept, MEDI-578, REGN475, Muromonab-CD3, Abiximab, Rituximab, Basiliximab, Palivizumab, Infliximab, Gemtuzumab ozogamicin, Ibritumomab tiuxetan, Adalimumab, Omalizumab, Tositumomab, Tositumomab-I131, Efalizumab, Abciximab, Certolizumab pegol, Eculizumab, AMG-162, Zanolimumab, MDX-010, Anti0MRSA mAb, Pexelizumab, Mepolizumab, Epratuzumab, Anti-RSV mAb, Afelimomab, Catumaxomab, WX-G250, and combinations thereof.

27. The method according to claim 21, wherein the administration of the agent which activates both of A1 and A2A adenosine receptors and the administration of the macromolecular therapeutic agent is systemic administration.

28. The method according to claim 21, wherein the administration of the agent which activates both of A1 and A2A adenosine receptors or the administration of the macromolecular therapeutic agent is systemic administration.

29. A method for treating a CNS disease, disorder, or condition in a subject, comprising administering to said subject (a) at least one agent which activates both of A1 and A2A adenosine receptors; and (b) a therapeutic agent.

30. The method according to claim 29, wherein the agent which activates both of A1 and A2A adenosine receptors is an agonist of both A1 and A2A receptors.

31. The method according to claim 30, wherein the agent which activates both of A1 and A2A adenosine receptors is a broad spectrum adenosine receptor agonist.

32. The method according to claim 30, wherein the agonist of both A1 and A2A receptors is AMP 579.

33. The method according to claim 31, wherein the agonist of both A1 and A2A receptors is NECA.

34. The method according to claim 29, wherein the therapeutic agent is a macromolecular therapeutic agent.

35. The method according to claim 34, wherein the macromolecular therapeutic agent is a monoclonal antibody.

36. The method according to claim 35, wherein the monoclonal antibody is selected from the group consisting of 6E10, PF-04360365, 131I-chTNT-1/B MAb, 131I-L19SIP, 177Lu-J591, ABT-874, AIN457, alemtuzumab, anti-PDGFR alpha monoclonal antibody IMC-3G3, astatine At 211 monoclonal antibody 81C6, Bapineuzumab, Bevacizumab, cetuximab, cixutumumab, Daclizumab, Hu MiK-beta-1, HuMax-EGFr, iodine I 131 monoclonal antibody 3F8, iodine I 131 monoclonal antibody 81C6, iodine I 131 monoclonal antibody 8H9, iodine I 131 monoclonal antibody TNT-1/B, LMB-7 immunotoxin, MAb-425, MGAWN1, Me1-14 F(ab′)2, M-T412, Natalizumab, Neuradiab, Nimotuzumab, Ofatumumab, Panitumumab, Ramucirumab, ranibizumab, SDZ MSL-109, Solanezumab, Trastuzumab, Ustekinumab, Zalutumumab, Tanezumab, Aflibercept, MEDI-578, REGN475, Muromonab-CD3, Abiximab, Rituximab, Basiliximab, Palivizumab, Infliximab, Gemtuzumab ozogamicin, Ibritumomab tiuxetan, Adalimumab, Omalizumab, Tositumomab, Tositumomab-I131, Efalizumab, Abciximab, Certolizumab pegol, Eculizumab, AMG-162, Zanolimumab, MDX-010, Anti0MRSA mAb, Pexelizumab, Mepolizumab, Epratuzumab, Anti-RSV mAb, Afelimomab, Catumaxomab, WX-G250, and combinations thereof.

37. The method according to claim 29, wherein the therapeutic agent is a small molecule therapeutic agent.

38. The method according to claim 37, wherein the small molecule therapeutic agent is selected from the group consisting of acetaminophen, acetylsalicylic acid, acyltransferase, alprazolam, amantadine, amisulpride, amitriptyline, amphetamine-dextroamphetamine, amsacrine, antipsychotics, antivirals, apomorphine, arimoclomol, aripiprazole, asenapine, aspartoacyclase enzyme, atomoxetine, atypical antipsychotics, azathioprine, baclofen, beclamide, benserazide, benserazide-levodopa, benzodiazepines, benztropine, bevacizumab, bleomycin, brivaracetam, bromocriptine, buprenorphine, bupropion, cabergoline, carbamazepine, carbatrol, carbidopa, carbidopa-levodopa, carboplatin, chlorambucil, chlorpromazine, chlorprothixene, cisplatin, citalopram, clobazam, clomipramine, clonazepam, clozapine, codeine, COX-2 inhibitors, cyclophosphamide, dactinomycin, dexmethylphenidate, dextroamphetaine, diamorphine, diastat, diazepam, diclofenac, donepezil, doxorubicin, droperidol, entacapone, epirubicin, escitalopram, ethosuximide, etoposide, felbamate, fluoxetine, flupenthixol, fluphenazine, fosphenytoin, gabapentin, galantamine, gamma hydroxybutyrate, gefitinib, haloperidol, hydantoins, hydrocordone, hydroxyzine, ibuprofen, ifosfamide, IGF-1, iloperidone, imatinib, imipramine, interferons, irinotecan, KNS-760704, lacosamide, lamotrigine, levetiracetam, levodopa, levomepromazine, lisdexamfetamine, lisuride, lithium carbonate, lypolytic enzyme, mechlorethamine, mGluR2 agonists, memantine, meperidine, mercaptopurine, mesoridazine, mesuximide, methamphetamine, methylphenidate, minocycline, modafinil, morphine, N-acetylcysteine, naproxen, nelfinavir, neurotrin, nitrazepam, NSAIDs, olanzapine, opiates, oseltamivir, oxaplatin, paliperidone, pantothenate kinase 2, Parkin, paroxetine, pergolide, periciazine, perphenazine, phenacemide, phenelzine, phenobarbitol, phenturide, phenyloin, pimozide, Pink1, piribedil, podophyllotoxin, pramipexole, pregabalin, primidone, prochlorperazine, promazine, promethazine, protriptyline, pyrimidinediones, quetiapine, rasagiline, remacemide, riluzole, risperidone, ritonavir, rituximab, rivastigmine, ropinirole, rotigotine, rufinamide, selective serotonin reuptake inhibitors (SSRIs), selegine, selegiline, sertindole, sertraline, sodium valproate, stiripentol, taxanes, temazepam, temozolomide, tenofovir, tetrabenazine, thiamine, thioridazine, thiothixene, tiagabine, tolcapone, topiramate, topotecan, tramadol, tranylcypromine, trastuzumab, tricyclic antidepressants, trifluoperazine, triflupromazine, trihexyphenidyl, trileptal, valaciclovir, valnoctamide, valproamide, valproic acid, venlafaxine, vesicular stomatitis virus, vigabatrin, vinca alkaloids, zanamivir, ziprasidone, zonisamide, zotepine, zuclopenthixol, and combinations thereof.

39. The method according to claim 29, wherein the CNS disease, disorder, or condition is a metabolic disease, a behavioral disorder, a personality disorder, dementia, a cancer, a neurodegenerative disorder, pain, a viral infection, a sleep disorder, a seizure disorder, acid lipase disease, Fabry disease, Wernicke-Korsakoff syndrome, ADHD, anxiety disorder, borderline personality disorder, bipolar disorder, depression, eating disorder, obsessive-compulsive disorder, schizophrenia, Alzheimer's disease, Barth syndrome and Tourette's syndrome, Canavan disease, Hallervorden-Spatz disease, Huntington's disease, Lewy Body disease, Lou Gehrig's disease, Machado-Joseph disease, Parkinson's disease, or Restless Leg syndrome.

40. The method according to claim 39, wherein the pain is neuropathic pain, central pain syndrome, somatic pain, visceral pain or headache.

41. A method for treating a CNS disease, disorder, or condition in a subject, comprising administering to said subject (a) an A1-selective adenosine receptor agonist; (b) an A2A-selective receptor agonist; and (c) a therapeutic agent.

42. The method according to claim 41, wherein the A1-selective adenosine receptor agonist and an A2A-selective receptor agonist are formulated in a single unit dosage form.

43. The method according to claim 41, wherein the A1-selective adenosine receptor agonist and an A2A-selective receptor agonist are administered simultaneously.

44. The method according to claim 41, wherein the A1-selective adenosine receptor agonist and an A2A-selective receptor agonist are administered sequentially.

45. The method according to claim 41, wherein the A1-selective adenosine receptor agonist is selected from the group consisting of CCPA, 8-cyclopentyl-1,3-dipropylxanthine, R-phenylisopropyl-adenosine, N6-Cyclopentyladenosine, N(6)-cyclohexyladenosine, and combinations thereof.

46. The method according to claim 41, wherein the A2A-selective receptor agonist is selected from the group consisting of Lexiscan, CGS 21680, ATL-146e, YT-146 (2-(1-octynyl)adenosine), DPMA (N6-(2-(3,5-dimethoxyphenyl)-2-(2-methylphenyl)ethyl)adenosine), and combinations thereof.

47. The method according to claim 41, comprising administering to the subject a composition comprising an A1 adenosine receptor agonist and an A2A adenosine receptor agonist, and a pharmaceutically acceptable carrier, excipient, or vehicle.

48. The method according to claim 41, wherein the therapeutic agent is a macromolecular therapeutic agent.

49. The method according to claim 48, wherein the macromolecular therapeutic agent is a monoclonal antibody.

50. The method according to claim 49, wherein the monoclonal antibody is selected from the group consisting of 6E10, PF-04360365, 131I-chTNT-1/B MAb, 131I-L19SIP, 177Lu-J591, ABT-874, AIN457, alemtuzumab, anti-PDGFR alpha monoclonal antibody IMC-3G3, astatine At 211 monoclonal antibody 81C6, Bapineuzumab, Bevacizumab, cetuximab, cixutumumab, Daclizumab, Hu MiK-beta-1, HuMax-EGFr, iodine I 131 monoclonal antibody 3F8, iodine I 131 monoclonal antibody 81C6, iodine I 131 monoclonal antibody 8H9, iodine I 131 monoclonal antibody TNT-1/B, LMB-7 immunotoxin, MAb-425, MGAWN1, Me1-14 F(ab′)2, M-T412, Natalizumab, Neuradiab, Nimotuzumab, Ofatumumab, Panitumumab, Ramucirumab, ranibizumab, SDZ MSL-109, Solanezumab, Trastuzumab, Ustekinumab, Zalutumumab, Tanezumab, Aflibercept, MEDI-578, REGN475, Muromonab-CD3, Abiximab, Rituximab, Basiliximab, Palivizumab, Infliximab, Gemtuzumab ozogamicin, Ibritumomab tiuxetan, Adalimumab, Omalizumab, Tositumomab, Tositumomab-I131, Efalizumab, Abciximab, Certolizumab pegol, Eculizumab, AMG-162, Zanolimumab, MDX-010, Anti0MRSA mAb, Pexelizumab, Mepolizumab, Epratuzumab, Anti-RSV mAb, Afelimomab, Catumaxomab, WX-G250, and combinations thereof.

51. The method according to claim 41, wherein the therapeutic agent is a small molecule therapeutic agent.

52. The method according to claim 51, wherein the small molecule therapeutic agent is selected from the group consisting of acetaminophen, acetylsalicylic acid, acyltransferase, alprazolam, amantadine, amisulpride, amitriptyline, amphetamine-dextroamphetamine, amsacrine, antipsychotics, antivirals, apomorphine, arimoclomol, aripiprazole, asenapine, aspartoacyclase enzyme, atomoxetine, atypical antipsychotics, azathioprine, baclofen, beclamide, benserazide, benserazide-levodopa, benzodiazepines, benztropine, bevacizumab, bleomycin, brivaracetam, bromocriptine, buprenorphine, bupropion, cabergoline, carbamazepine, carbatrol, carbidopa, carbidopa-levodopa, carboplatin, chlorambucil, chlorpromazine, chlorprothixene, cisplatin, citalopram, clobazam, clomipramine, clonazepam, clozapine, codeine, COX-2 inhibitors, cyclophosphamide, dactinomycin, dexmethylphenidate, dextroamphetaine, diamorphine, diastat, diazepam, diclofenac, donepezil, doxorubicin, droperidol, entacapone, epirubicin, escitalopram, ethosuximide, etoposide, felbamate, fluoxetine, flupenthixol, fluphenazine, fosphenytoin, gabapentin, galantamine, gamma hydroxybutyrate, gefitinib, haloperidol, hydantoins, hydrocordone, hydroxyzine, ibuprofen, ifosfamide, IGF-1, iloperidone, imatinib, imipramine, interferons, irinotecan, KNS-760704, lacosamide, lamotrigine, levetiracetam, levodopa, levomepromazine, lisdexamfetamine, lisuride, lithium carbonate, lypolytic enzyme, mechlorethamine, mGluR2 agonists, memantine, meperidine, mercaptopurine, mesoridazine, mesuximide, methamphetamine, methylphenidate, minocycline, modafinil, morphine, N-acetylcysteine, naproxen, nelfinavir, neurotrin, nitrazepam, NSAIDs, olanzapine, opiates, oseltamivir, oxaplatin, paliperidone, pantothenate kinase 2, Parkin, paroxetine, pergolide, periciazine, perphenazine, phenacemide, phenelzine, phenobarbitol, phenturide, phenyloin, pimozide, Pink1, piribedil, podophyllotoxin, pramipexole, pregabalin, primidone, prochlorperazine, promazine, promethazine, protriptyline, pyrimidinediones, quetiapine, rasagiline, remacemide, riluzole, risperidone, ritonavir, rituximab, rivastigmine, ropinirole, rotigotine, rufinamide, selective serotonin reuptake inhibitors (SSRIs), selegine, selegiline, sertindole, sertraline, sodium valproate, stiripentol, taxanes, temazepam, temozolomide, tenofovir, tetrabenazine, thiamine, thioridazine, thiothixene, tiagabine, tolcapone, topiramate, topotecan, tramadol, tranylcypromine, trastuzumab, tricyclic antidepressants, trifluoperazine, triflupromazine, trihexyphenidyl, trileptal, valaciclovir, valnoctamide, valproamide, valproic acid, venlafaxine, vesicular stomatitis virus, vigabatrin, vinca alkaloids, zanamivir, ziprasidone, zonisamide, zotepine, zuclopenthixol, and combinations thereof.

53. The method according to claim 41, wherein the CNS disease, disorder, or condition is a metabolic disease, a behavioral disorder, a personality disorder, dementia, a cancer, a neurodegenerative disorder, pain, a viral infection, a sleep disorder, a seizure disorder, acid lipase disease, Fabry disease, Wernicke-Korsakoff syndrome, ADHD, anxiety disorder, borderline personality disorder, bipolar disorder, depression, eating disorder, obsessive-compulsive disorder, schizophrenia, Alzheimer's disease, Barth syndrome and Tourette's syndrome, Canavan disease, Hallervorden-Spatz disease, Huntington's disease, Lewy Body disease, Lou Gehrig's disease, Machado-Joseph disease, Parkinson's disease, or Restless Leg syndrome.

54. The method according to claim 53, wherein the pain is neuropathic pain, central pain syndrome, somatic pain, visceral pain or headache.

55. A method of temporarily increasing the permeability of the blood brain barrier of a subject comprising:
selecting a subject in need of a temporary increase in permeability of the blood brain barrier;
providing an agent which activates either the A1 or the A2A adenosine receptor; and
administering to the selected subject either the A1 or the A2A adenosine receptor activating agent under conditions effective to temporarily increase the permeability of the blood brain barrier.

56.-67. (canceled)

68. A method for decreasing blood brain barrier permeability in a subject comprising administering to said subject an agent which blocks or inhibits A2A signaling.

69.-70. (canceled)

71. A method of remodeling an actin cytoskeleton of a blood brain barrier endothelial cell, said method comprising contacting said endothelial cell with an agent which activates both of A1 and A2A adenosine receptors.

72.-78. (canceled)
